(19) United States
(12) Patent Application Publication
(10) Pub. No.: US 2016/0374700 A1
(43) Pub. Date: Dec. 29, 2016

(54) Endoscope Device and Method of Use

(71) Applicant: Endovate LLC, Scottsdale, AZ (US)

(72) Inventors: Kevin W. Olden, Scottsdale, AZ (US); John David Washington, Scottsdale, AZ (US)

(73) Assignee: Endovate LLC, Scottsdale, AZ (US)

(21) Appl. No.: 15/192,985

(22) Filed: Jun. 24, 2016

Related U.S. Application Data
(60) Provisional application No. 62/185,556, filed on Jun. 26, 2015.

(51) Int. Cl. A61B 17/22; A61B 17/00

(52) U.S. Cl. CPC A61B 17/22; A61B 17/00234; A61B 2017/22035; A61B 2017/22079; A61B 2017/00561; A61B 2017/00296

(57) ABSTRACT

The present specification discloses an endoscope device comprising a body having a proximal opening in communication with an opposite distal opening, and the body having a nose substantially about the distal opening, a primary lumen or blockage receiving lumen formed within the body through which suction is selectively applied, the primary lumen being contiguous with the distal opening, and a secondary lumen or endoscope receiving lumen formed within the body so as to be in communication with the primary lumen, the secondary lumen being contiguous with the proximal opening and configured for receipt of an endoscope, whereby suction applied through a suction port of the endoscope when positioned in the secondary lumen applies suction to the primary lumen.

In gastroenterology practice and in emergency room and other such cases, it is not uncommon for a patient to present with a partial or complete blockage of the esophagus, as by a foreign object or a food impaction, causing pain, nausea or vomiting, loss of appetite, and other such symptoms. Clinical challenges related to assessment and non-surgical treatment of such esophageal conditions call for improved devices and methods particularly relating to endoscopy.

The most common form of esophageal blockage is generally understood to be a food impaction such as meat bolus. Treatment options for such food bolus impactions generally fall into two categories: removal or extraction (“pull technique”); or advancement of the bolus into the stomach (“push technique”). Visualization of the food impaction and surrounding anatomy proximal and distal of the impaction is an important aspect of diagnosis and treatment. Visualization throughout the selected treatment is preferable. As such, endoscopic techniques are widely used.

Esophageal food impaction can have a variety of causes, determination of which plays a further role in ultimately determining the recommended treatment. Sometimes the meat or other food bolus is simply too large and/or not sufficiently chewed, and being substantially solid or semi-solid, it has become lodged in the esophagus, often just above the lower esophageal sphincter. In other cases, some esophageal pathology such as lesions or tumors, scarring, Schatzki rings, predisposing peptic stricture, or other such esophageal condition or abnormality is responsible for the food impaction due to effectively obstructing or narrowing the esophagus. In any such cases, the food impaction may thus be solid or semi-solid but is most often gooey, semi-digested food, sometimes amounting to on the order of 1-2 cups.

Removal or extraction of such common food impactions presents specific clinical challenges, particularly when repeated insertions of an endoscope or other such device can increase the risk of patient trauma and post-procedure discomfort. Concerns regarding esophageal perforation and aspiration (material passing into the trachea and lungs), either of which could be life-threatening, have led to the development and use of a number of related endoscopic devices such as overtubes and protective hoods, but these devices have their own limitations and drawbacks and ultimately do not address the issue of more efficiently removing the impaction while under substantially consistent visualization. And where it has been determined that an anatomical condition or pathology has caused the food impaction, this will often rule out some treatment options, including for many practitioners any “push technique” for fear of trauma to the likely already compromised anatomy.

Other related approaches to attempting to treat a food impaction without surgery have shortcomings as well, such as the medical or pharmacological approach of relaxing the esophagus through the administration of glucagon or other agent, which carries the risk of reflux and aspiration into the trachea and lungs, or the introduction of enzyme compounds in an attempt to dissolve the blockage, which simply adds time and uncertainty to the procedure, the hope being with either of these pharma approaches that the impaction will pass on its own if given enough time under the right conditions (which assumes that the pathology can ultimately accommodate passage of the impaction). Insufflation, or attempting to effectively inflate or expand the esophagus with air so as to dilate the esophagus and allow the impaction to pass, also involves risks to what could be already compromised anatomy as well as presenting similar concerns regarding reflux and aspiration. Finally, devices employed in connection with foreign body removal from the esophagus or stomach such as Roth or Dormia baskets, nets, polypectomy snares, alligator, rat-tooth, or shark-tooth forceps or graspers, and magnetic retrievers are most often simply incapable of extracting the semi-digested, mushy food impaction typically encountered.

What the clinician is effectively left with in treating the most common type of esophageal impaction (food bolus or the like) is suction, typically through the suction port of the endoscope itself. However, the typical 2-4 mm suction channel of the average gastroscope is woefully inadequate to remove a relatively large volume food impaction of on the order of 1-2 cups and is prone to clogging. Even experienced practitioners may be required using such standard equipment to make 30-40 passes (insertions) over the course of on the order of 2 hours to completely remove the impaction, which again can lead to at least patient soreness and extended time under sedation as well as potential trauma to the esophagus, such as damage to the mucosa or actual perforation. What is needed is a device, in the form of an endoscope attachment or a purpose-built endoscope that enables more effective removal of esophageal food impactions.

Furthermore, even in the case of foreign object removal, an improved endoscope device and method can equip the clinician to more effectively and safely extract such an object non-surgically. In some contexts, the blockage may involve both a foreign object and food impaction, such that an endoscope device that is better equipped to manage both kinds of blockage, or a combination thereof, can effectively enable more efficient removal by the clinician.

### SUMMARY

Aspects of the present invention teach certain benefits in construction and use which give rise to the exemplary advantages described below.

Aspects of the present specification provide an endoscope device comprising a body having a proximal opening and an opposite distal opening, the proximal and distal openings being in fluid communication, and the body having a nose or leading edge substantially about the distal opening, a primary lumen or blockage receiving lumen formed within the body through which suction is selectively applied, and a secondary lumen or endoscope receiving lumen formed within the body so as to be in communication with the primary lumen, the secondary lumen being contiguous with the proximal opening and configured for frictional receipt of an endoscope, whereby suction applied through a suction port of the endoscope when positioned in the secondary lumen applies suction to the primary lumen.

Other aspects of the present specification provide an endoscope device with an enlarged primary lumen for relatively greater retrieval per suction event.

Other aspects of the present specification provide a primary lumen that is at least partially offset from and adjacent to the secondary lumen for improved visualization during the suction event.

Other aspects of the present specification provide a suction channel within the endoscope device so as to apply suction from the secondary lumen to a proximal location within the primary lumen.

Other aspects of the present specification provide a resilient retention lip or funnel formed in the distal opening of the endoscope device.

Other aspects of the present specification provide a screen or filter member positioned within the endoscope device between the proximal and distal lumens.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 illustrates a perspective view of an exemplary endoscope device, in accordance with at least one embodiment.

FIG. 2 illustrates a reduced scale side cross-sectional view thereof, in accordance with at least one embodiment.

FIG. 3 illustrates an alternative reduced scale side cross-sectional view thereof, in accordance with at least one embodiment.

FIG. 4 illustrates a further alternative reduced scale side cross-sectional view thereof, in accordance with at least one embodiment.

FIG. 5 illustrates a further alternative reduced scale side cross-sectional view thereof, in accordance with at least one embodiment.

FIG. 6 illustrates a reduced scale perspective view thereof installed on the distal end of an endoscope, in accordance with at least one embodiment.

FIG. 7A illustrates the assembly of the device on an endoscope as shown in FIG. 6 at a first location within the esophagus.

FIG. 7B illustrates the assembly of the device on an endoscope as shown in FIG. 6 now at a second location within the esophagus, the device being in a first mode of operation.

FIG. 7C illustrates the assembly of the device on an endoscope as shown in FIG. 6 still at the second location within the esophagus of FIG. 7B, the device now being in a second mode of operation.

FIG. 8 illustrates a perspective view of an alternative exemplary endoscope device, in accordance with at least one embodiment.

FIG. 9 illustrates an end view thereof, in accordance with at least one embodiment.

FIG. 10A illustrates a side cross-sectional view thereof, in accordance with at least one embodiment.

FIG. 10B illustrates a side cross-sectional view thereof now with an endoscope inserted therein, in accordance with at least one embodiment.

FIG. 10C illustrates a side cross-sectional view thereof with an endoscope inserted therein as shown in FIG. 10B, now in use within the esophagus.

FIG. 11A illustrates a side cross-sectional view of an alternative exemplary endoscope device with an endoscope inserted therein and in a first mode of operation, in accordance with at least one embodiment.

FIG. 11B illustrates a side cross-sectional view thereof still with an endoscope inserted therein and now in a second mode of operation, in accordance with at least one embodiment.

FIG. 12 illustrates a side cross-sectional view of a further alternative exemplary endoscope device, in accordance with at least one embodiment.

FIG. 13 illustrates an end view of a still further alternative exemplary endoscope device, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DEATAILED DESCRIPTION

The present specification relates generally to a device that would attach to an endoscope or be formed as a “purpose-built scope” to assist a G.I. doctor or other clinician in visualizing and removing a “foreign body” or “food impaction” or “bolus” from a patient's esophagus, as when a patient has swallowed something too hard or large to pass into their stomach that has instead become lodged in the esophagus or when the patient has some anatomical issue within the esophagus that has caused a restriction, in either case the obstruction needing to be safely and preferably non-surgically removed. According to aspects of the present invention, the endoscope device enables food impaction or foreign body removal using suction in a new and improved way, basically allowing for better visualization during the procedure and retention of larger amounts of material per pass (per intubation), thereby reducing the risk of patient trauma (e.g., perforation of the esophagus) or discomfort during and after the procedure. While several exemplary embodiments of such an endoscope device are shown and described, it will be appreciated by those skilled in the art that a variety of other such devices according to aspects of the present invention are possible and that features may be differently combined or substituted employing any related technologies now known or later developed without departing from the spirit and scope of the invention.

The present specification further addresses the shortcomings of known devices and methods for non-surgical treatment or management of esophageal food impactions and foreign bodies. The present specification discloses an endoscope device comprising a body having a proximal opening and an opposite distal opening, the proximal and distal openings being in fluid communication, and the body having a nose substantially about the distal opening, a primary lumen formed within the body through which suction is selectively applied, the primary lumen being contiguous with the distal opening, and a secondary lumen formed within the body so as to be in fluid communication with the primary lumen, the secondary lumen being contiguous with the proximal opening and configured for frictional receipt of an endoscope, whereby suction applied through a suction port of the endoscope when positioned in the secondary lumen applies suction to the primary lumen. The endoscope device has a larger distal volume so as to not only contain a relatively larger amount of food impaction per insertion or suction event but to also retain and effectively shield a foreign body, acquisition and removal of the foreign body along with the scope can be safely performed without the use of an overtube or hood.

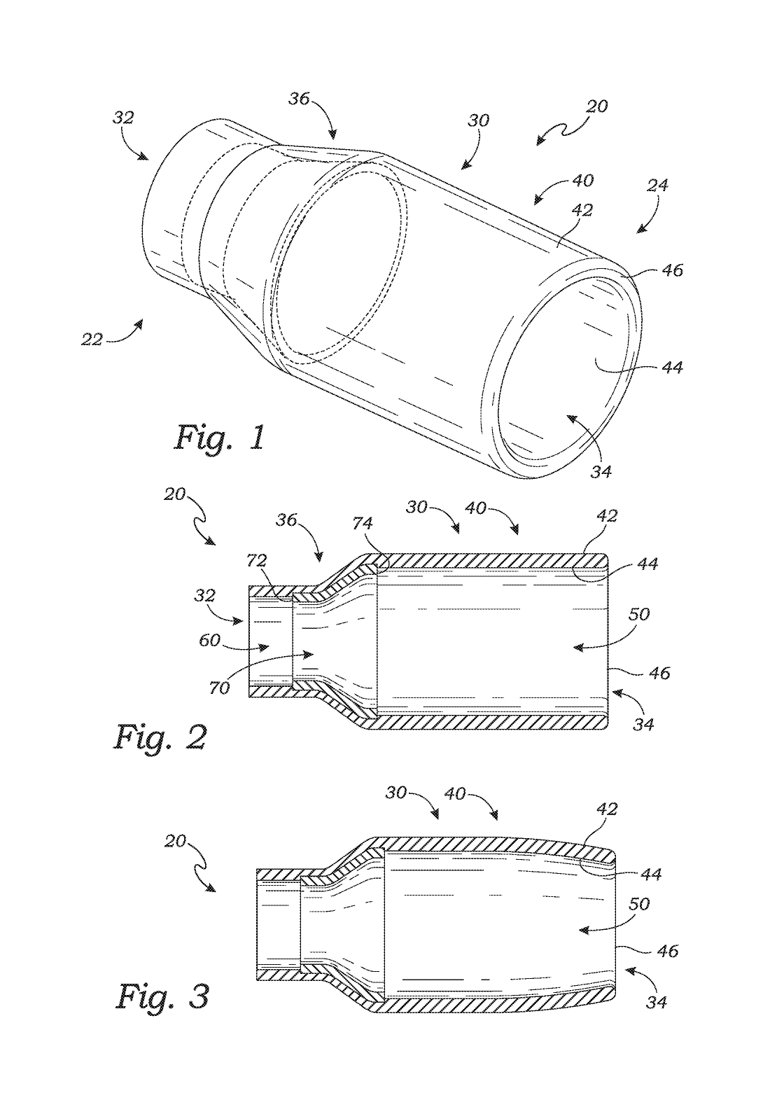

Referring first to FIG. 1, there is shown a perspective view of an exemplary endoscope device or attachment 20, in accordance with at least one embodiment, configured for selective or removable attachment on the distal end of an endoscope N (FIG. 6). The endoscope device 20 generally comprises a body 30 extending between a proximal end 22 and a distal end 24 and having a proximal opening 32 and an opposite distal opening 34, respectively. The body 30 is defined by a body wall 40 having an outer surface 42 and an opposite inner surface 44 with the distance between defining the effective wall thickness of the body wall 40, more about which is said below. Within the body 30 of the endoscope device 20 there are formed a blockage receiving lumen or primary lumen 50 associated with suction or retrieval, acting as a blockage receiving means, and an endoscope receiving lumen or secondary lumen 60 associated with the endoscope N acting as an endoscope receiving means, more about which is said below. By way of overview, the primary lumen 50 is configured for suction to be selectively applied therethrough and is contiguous with the distal opening 34, while the secondary lumen 60 is formed within the body 30 so as to be in fluid communication with the primary lumen 50 and is contiguous with the proximal opening 32 and configured for frictional receipt, or other form of attachment, of an endoscope N, whereby suction selectively applied through a suction port of the endoscope N when positioned in the secondary lumen 60 applies suction to the primary lumen 50. As shown, the device 20 may be generally proportioned so as to be longer than it is wide, and in one embodiment may be scaled such that its length is approximately double its width. In the exemplary embodiment, the endoscope device 20 is substantially annular, having a relatively larger diameter at its distal end 24 associated with a distal opening 34 through which a food impaction or other material may be suctioned within the device 20, in whole or in part, and a relatively smaller, tapered-down diameter at its proximal end 22 associated with a proximal opening 32 through which the distal end of the endoscope N is inserted when the device 20 is configured as an attachment to the endoscope N. Accordingly, a step 36 is formed between the proximal and distal ends 22, 24 of the device 20. Those skilled in the art should understand that all figures in the present application are schematic in nature and fundamentally that none of the figures are to be taken literally or to scale, each such figure being simply illustrative of features and aspects of the present invention and non-limiting. While the exemplary device 20 is shown as being annular, this is not necessarily the case, as other geometries such as elliptical may also be employed in at least portions of the endoscope device 20. Where the device 20 or any feature thereof is annular or round, it will be appreciated that any reference to “width” is to be understood as the feature's diameter.

Turning to FIG. 2, there is shown a reduced scale side cross-sectional view of the endoscope device 20 of FIG. 1. As illustrated, the device 20 may be formed of at least two components: the body 30 over-molded or otherwise formed or installed over an interior fitting 70, in which example the fitting 70 cooperates in forming the step 36. Such components may be formed of the same or different material depending on aspects of the invention and its intended use. In the exemplary embodiment, the interior fitting 70 may be formed of a relatively rigid plastic, polymer, rubber, elastomer, resin, or the like such as relatively hard or high durometer polyvinyl or polyvinyl chloride (“PVC”), polyethylene or high density polyethylene (“HDPE”), polypropylene, polyurethane, polycarbonate, acrylic, acrylonitrile butadiene styrene (“ABS”), nylon, polylactic acid (“PLA”), polybenzimidazole, polyether sulfone, polyether ether ketone, polyetherimide, polyphenylene oxide, polyphenylene sulfide, polystyrene, Teflon, silicone rubber, or any other such thermoplastic or like material now known or later developed. By way of illustration, the hardness of the interior fitting 70 may be in the range of 25-100 Shore A durometer.

An endoscope device 20 disclosed herein has a material of construction for the interior fitting 70 adequate to maintain its shape and provide mechanical integrity to the surrounding body 30 and the overall device 20. In aspects of this embodiment, an endoscope device 20 disclosed herein has an interior fitting 70 having a Shore A durometer hardness of, e.g., about 25, about 30, about 35, about 40, about 45, about 50, about 55, about 60, about 65, about 70, about 75, about 80, about 85, about 90, about 95, or about 100. In other aspects of this embodiment, an endoscope device 20 disclosed herein has an interior fitting 70 having a Shore A durometer hardness of, e.g., at least 25, at least 30, at least 35, at least 40, at least 45, at least 50, at least 55, at least 60, at least 65, at least 70, at least 75, at least 80, at least 85, at least 90, at least 95, or at least 100. In other aspects of this embodiment, an endoscope device 20 disclosed herein has an interior fitting 70 having a Shore A durometer hardness of, e.g., at most 25, at most 30, at most 35, at most 40, at most 45, at most 50, at most 55, at most 60, at most 65, at most 70, at most 75, at most 80, at most 85, at most 90, at most 95, or at most 100.

In still other aspects of this embodiment, an endoscope device 20 disclosed herein has an interior fitting 70 having a Shore A durometer hardness of, e.g., about 25 to about 30, about 25 to about 35, about 25 to about 40, about 25 to about 45, about 25 to about 50, about 25 to about 55, about 25 to about 60, about 25 to about 65, about 25 to about 70, about 25 to about 75, about 25 to about 80, about 25 to about 85, about 25 to about 90, about 25 to about 95, about 25 to about 100, about 30 to about 35, about 30 to about 40, about 30 to about 45, about 30 to about 50, about 30 to about 55, about 30 to about 60, about 30 to about 65, about 30 to about 70, about 30 to about 75, about 30 to about 80, about 30 to about 85, about 30 to about 90, about 30 to about 95, about 30 to about 100, about 35 to about 40, about 35 to about 45, about 35 to about 50, about 35 to about 55, about 35 to about 60, about 35 to about 65, about 35 to about 70, about 35 to about 75, about 35 to about 80, about 35 to about 85, about 35 to about 90, about 35 to about 95, about 35 to about 100, about 40 to about 45, about 40 to about 50, about 40 to about 55, about 40 to about 60, about 40 to about 65, about 40 to about 70, about 40 to about 75, about 40 to about 80, about 40 to about 85, about 40 to about 90, about 40 to about 95, about 40 to about 100, about 45 to about 50, about 45 to about 55, about 45 to about 60, about 45 to about 65, about 45 to about 70, about 45 to about 75, about 45 to about 80, about 45 to about 85, about 45 to about 90, about 45 to about 95, about 45 to about 100, about 50 to about 55, about 50 to about 60, about 50 to about 65, about 50 to about 70, about 50 to about 75, about 50 to about 80, about 50 to about 85, about 50 to about 90, about 50 to about 95, about 50 to about 100, about 55 to about 60, about 55 to about 65, about 55 to about 70, about 55 to about 75, about 55 to about 80, about 55 to about 85, about 55 to about 90, about 55 to about 95, about 55 to about 100, about 60 to about 65, about 60 to about 70, about 60 to about 75, about 60 to about 80, about 60 to about 85, about 60 to about 90, about 60 to about 95, about 60 to about 100, about 65 to about 70, about 65 to about 75, about 65 to about 80, about 65 to about 85, about 65 to about 90, about 65 to about 95, about 65 to about 100, about 70 to about 75, about 70 to about 80, about 70 to about 85, about 70 to about 90, about 70 to about 95, about 70 to about 100, about 75 to about 80, about 75 to about 85, about 75 to about 90, about 75 to about 95, about 75 to about 100, about 80 to about 85, about 80 to about 90, about 80 to about 95, about 80 to about 100, about 85 to about 90, about 85 to about 95, about 85 to about 100, about 90 to about 95, about 90 to about 100, or about 95 to about 100.

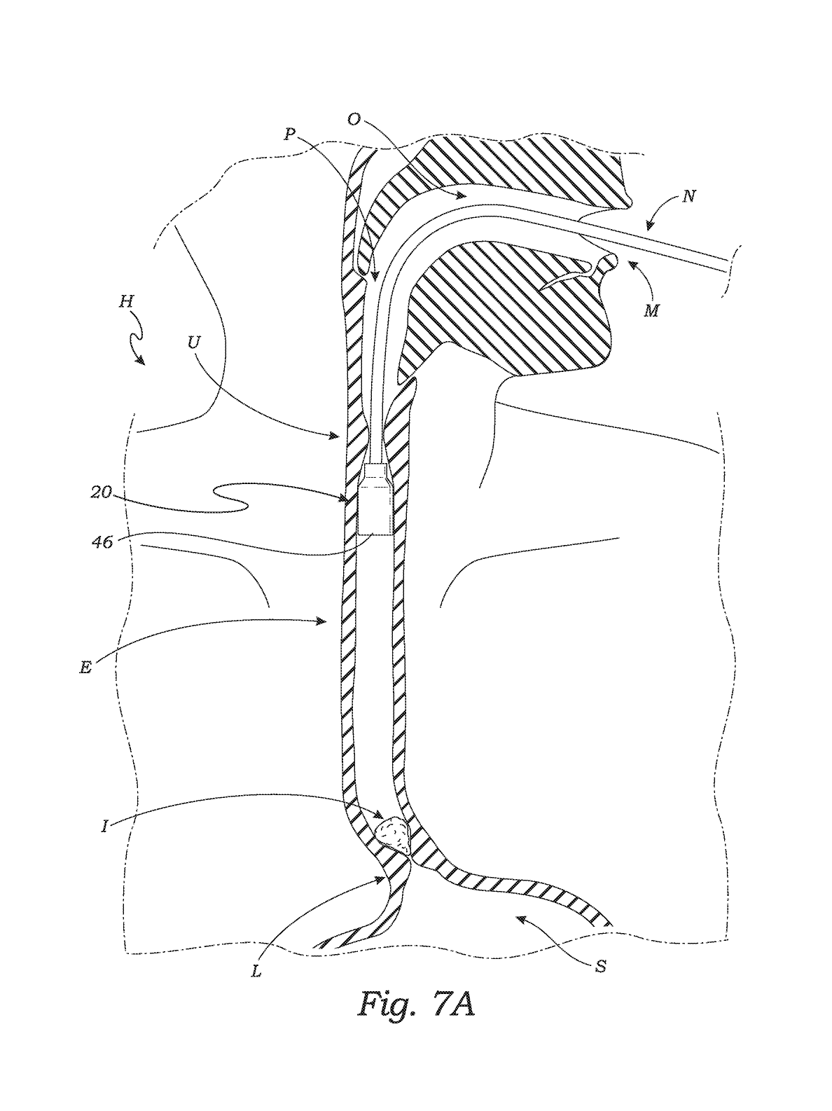

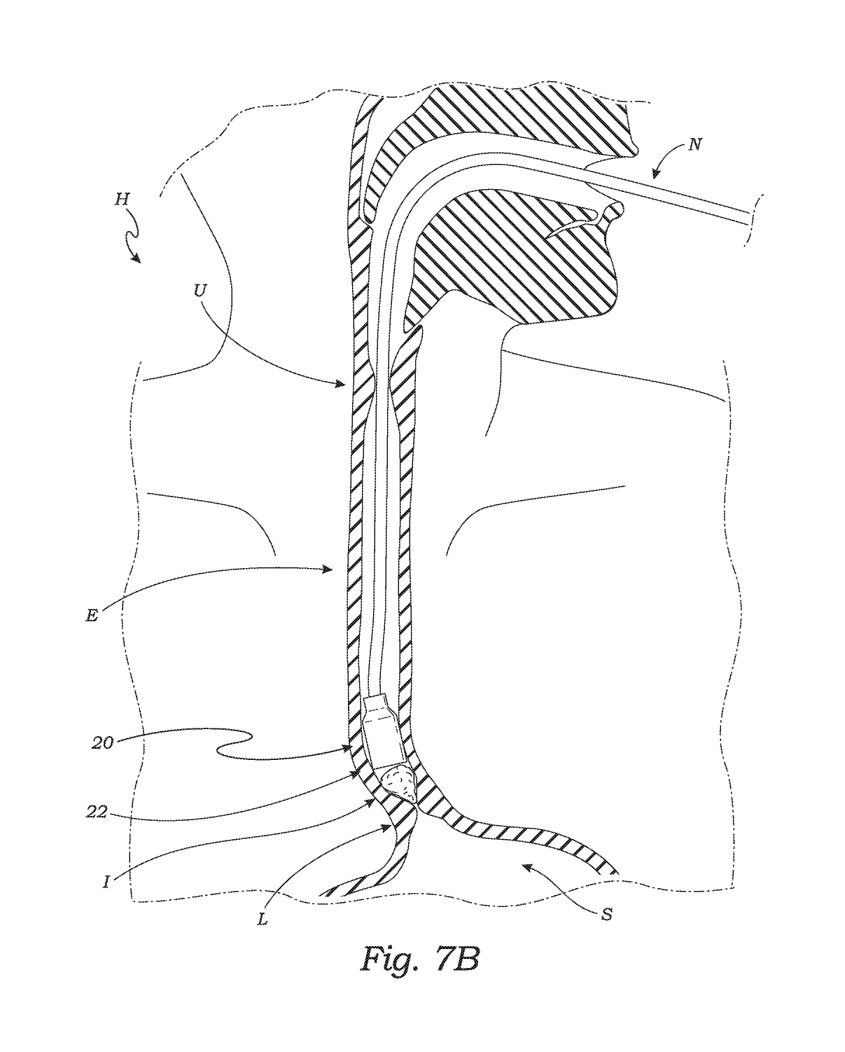

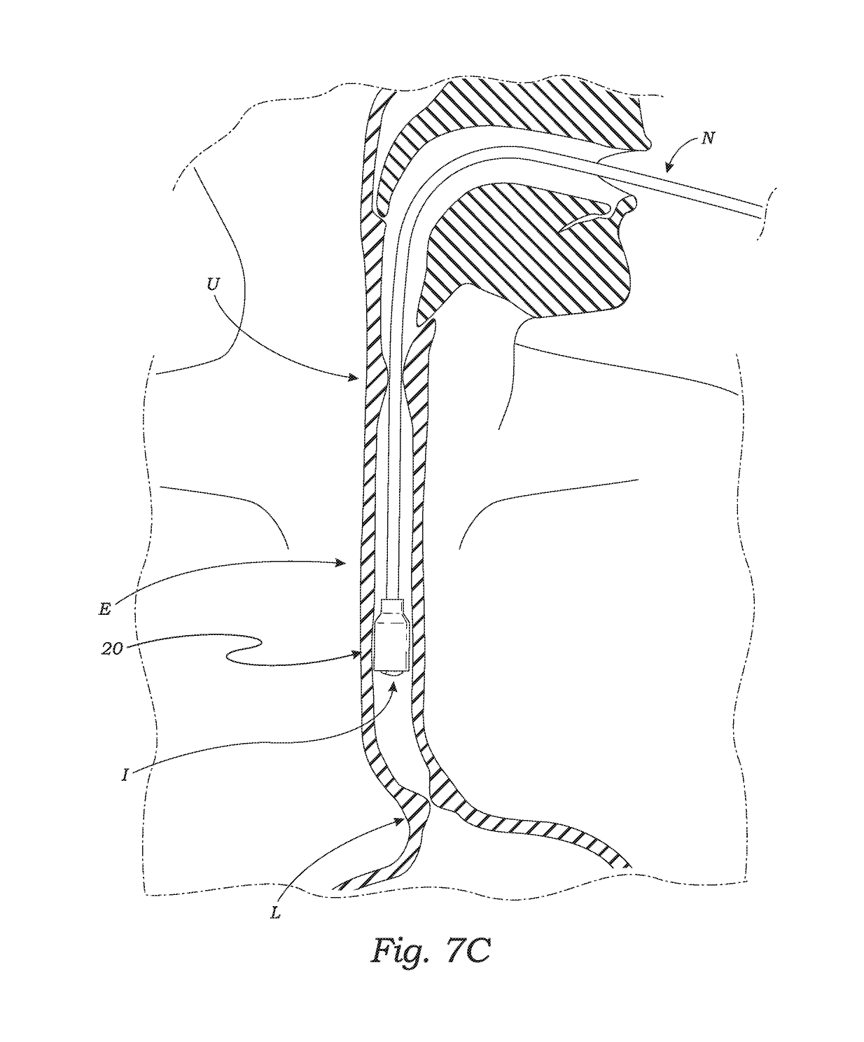

With continued reference to FIG. 2, the body 30 of the endoscope device 20 may again be formed of an identical or similar material to that of the interior fitting 70 or of a different material, integrally or separately. Moreover, the body 30 itself may be formed of multiple components or materials in order to suit particular design objectives. Notably, the proximal end 22 of the device 20 is configured for acceptance of and retention on an endoscope N (FIG. 6) as through a net- or interference- or press-fit arrangement (i.e., a frictional fit). Accordingly, in the exemplary embodiment, the body 30 particularly at its proximal end or at or adjacent the proximal opening 32 is to be relatively soft and pliable and even somewhat “gummy” so as to allow a good frictional engagement between the outside diameter of the endoscope N and the inside diameter of the proximal opening 32 as set forth further below. At the distal end 24 of the device 20 it is desirable that the body 30 also be somewhat pliable or flexible, but not necessarily to the same extent as the proximal end 22, as it is also important that the otherwise unsupported distal opening 34 have integrity and substantially maintain its intended or “at rest” shape during use so as to selectively open or expand the anatomy and provide clear access for suctioning and even maintain its shape during suctioning, more about which is said below regarding the device 20 in use, particularly in connection with FIGS. 7A-7C. This difference in performance or function of the body 30, and the body wall 40 specifically, from end to end or along its length can be achieved through the incorporation of different materials having varying mechanical properties or dimensionally as by increasing or decreasing wall thickness along the body 30, or both. In the exemplary embodiment as best shown in FIG. 2, the body 30 of the device 20 is formed of a single material with two different wall thicknesses: a relatively thinner wall at the proximal end 22 and a relatively thicker wall at the distal end 24. By way of further illustration, the body 30 may be formed of a relatively soft plastic, polymer, rubber, elastomer, resin or the like such as relatively soft or low durometer polyvinyl or polyvinyl chloride (“PVC”), polyethylene or high density polyethylene (“HDPE”), polypropylene, polyurethane, polycarbonate, acrylic, acrylonitrile butadiene styrene (“ABS”), nylon, polylactic acid (“PLA”), polybenzimidazole, polyether sulfone, polyether ether ketone, polyetherimide, polyphenylene oxide, polyphenylene sulfide, polystyrene, Teflon, silicone rubber, or any other such thermoplastic or like material now known or later developed. By way of illustration, the hardness of the body 30 may be in the range of 10-80 Shore A durometer. Moreover, to further aid in intubation, particularly with relatively larger sizes of the device 20, it is contemplated that a lubrous material or release agent may be incorporated or impregnated within or applied to particularly the outer surface 42 of the body wall 40.

An endoscope device 20 disclosed herein has a material of construction for the body 30 adequate to maintain its shape while having sufficient pliability or flexibility as part of the overall device 20. In aspects of this embodiment, an endoscope device 20 disclosed herein has a body 30 having a Shore A durometer hardness of, e.g., about 10, about 15, about 20, about 25, about 30, about 35, about 40, about 45, about 50, about 55, about 60, about 65, about 70, about 75, or about 80. In other aspects of this embodiment, an endoscope device 20 disclosed herein has a body 30 having a Shore A durometer hardness of, e.g., at least 10, at least 15, at least 20, at least 25, at least 30, at least 35, at least 40, at least 45, at least 50, at least 55, at least 60, at least 65, at least 70, at least 75, or at least 80. In other aspects of this embodiment, an endoscope device 20 disclosed herein has a body 30 having a Shore A durometer hardness of, e.g., at most 10, at most 15, at most 20, at most 25, at most 30, at most 35, at most 40, at most 45, at most 50, at most 55, at most 60, at most 65, at most 70, at most 75, or at most 80.

In still other aspects of this embodiment, an endoscope device 20 disclosed herein has a body 30 having a Shore A durometer hardness of, e.g., about 10 to about 15, about 10 to about 20, about 10 to about 25, about 10 to about 30, about 10 to about 35, about 10 to about 40, about 10 to about 45, about 10 to about 50, about 10 to about 55, about 10 to about 60, about 10 to about 65, about 10 to about 70, about 10 to about 75, about 10 to about 80, about 15 to about 20, about 15 to about 25, about 15 to about 30, about 15 to about 35, about 15 to about 40, about 15 to about 45, about 15 to about 50, about 15 to about 55, about 15 to about 60, about 15 to about 65, about 15 to about 70, about 15 to about 75, about 15 to about 80, about 20 to about 25, about 20 to about 30, about 20 to about 35, about 20 to about 40, about 20 to about 45, about 20 to about 50, about 20 to about 55, about 20 to about 60, about 20 to about 65, about 20 to about 70, about 20 to about 75, about 20 to about 80, about 25 to about 30, about 25 to about 35, about 25 to about 40, about 25 to about 45, about 25 to about 50, about 25 to about 55, about 25 to about 60, about 25 to about 65, about 25 to about 70, about 25 to about 75, about 25 to about 80, about 30 to about 35, about 30 to about 40, about 30 to about 45, about 30 to about 50, about 30 to about 55, about 30 to about 60, about 30 to about 65, about 30 to about 70, about 30 to about 75, about 30 to about 80, about 35 to about 40, about 35 to about 45, about 35 to about 50, about 35 to about 55, about 35 to about 60, about 35 to about 65, about 35 to about 70, about 35 to about 75, about 35 to about 80, about 40 to about 45, about 40 to about 50, about 40 to about 55, about 40 to about 60, about 40 to about 65, about 40 to about 70, about 40 to about 75, about 40 to about 80, about 45 to about 50, about 45 to about 55, about 45 to about 60, about 45 to about 65, about 45 to about 70, about 45 to about 75, about 45 to about 80, about 50 to about 55, about 50 to about 60, about 50 to about 65, about 50 to about 70, about 50 to about 75, about 50 to about 80, about 55 to about 60, about 55 to about 65, about 55 to about 70, about 55 to about 75, about 55 to about 80, about 60 to about 65, about 60 to about 70, about 60 to about 75, about 60 to about 80, about 65 to about 70, about 65 to about 75, about 65 to about 80, about 70 to about 75, about 70 to about 80, or about 75 to about 80.

With continued reference to FIG. 2, it can be seen that in the exemplary embodiment the interior fitting 70 of the device 20 is formed having an inside diameter that at its proximal end is smaller than the inside diameter of the proximal opening 32 or secondary lumen 60 of the device 20 and that at its distal end is smaller than the inside diameter of the distal opening 34 or primary lumen 50 of the device 20. The result is that a proximal shoulder 72 that faces proximally and a distal shoulder 74 that faces distally are both formed within the body 30 at opposite ends of the fitting 70. In alternative embodiments there could be formed one or the other or neither. Where a proximal shoulder 72 is formed, it will be appreciated that when an endoscope N (FIG. 6) is inserted within the proximal opening 32 of the body 30 in a substantially net fit arrangement, the proximal shoulder 72 forms a mechanical stop for the scope N to physically prevent its further advancement distally relative to the device 20, the shoulder 72 basically engaging the perimeter of the distal end of the scope N without blocking or interfering with the operation or operable use of any of the scope's channels. In other embodiments, again, there would be no such proximal shoulder 72 as would be the case where there is not interior fitting 70 or where the inside diameter of the interior fitting 70 is substantially the same the inside diameter of the proximal opening 32 of the body 30, resulting in a substantially continuous secondary lumen 60 into which the scope N selectively passes, relying on the frictional engagement between the scope N and body 30 to maintain the position of the device 20 relative to the endoscope N. Similarly, while a distal shoulder 74 is shown as being formed by the interior fitting 70, use of which in at least one alternative embodiment will be further appreciated in connection with the below discussion regarding FIG. 5, it is to be understood that such distal shoulder 74 may again instead not be present and the inner surface 44 of the body 30 transition somewhat smoothly or continuously from the proximal opening 32 to the distal opening 34 through the interior fitting 70 or other taper to form step 36. Regardless, it will be appreciated that the incorporation or inclusion of the interior fitting 70 within the body 30 provides further structural or mechanical support in the transitional region or step 36, illustrated here as an annular chamfer, where the device body 30 tapers from its relatively larger distal end 24 to its relatively smaller proximal end 22, particularly where the interior fitting 70 is formed of a relatively harder or more rigid material than the surrounding body 30. However, as above, it will be further appreciated that such could be accomplished instead or in addition by increasing wall thickness in the region, such that incorporation of a separate interior fitting 70 is understood to be illustrative and non-limiting. Even the proximal and distal shoulders 72, 74 may be formed within a unitary body 30 as desired through molding and other such fabrication methods now known or later developed rather than by an interior fitting 70 or the like. Furthermore, whether such interior fitting 70 is employed, any stop or proximal shoulder 72 may alternatively be formed so as to not only contact the outer perimeter edge of the distal end of the scope N but actually cover one or more of the scope's ports so as to effectively continue or extend such port to the distal end 24 of the device 20 as by forming a channel or bore within the body wall 40 that would communicate between the proximal shoulder 72, and thus the scope N, and the nose 46 or some other distal location along the wall 40 of the device 20. In this way, visualization or lighting, for example, may be extended beyond the distal end of the scope and thus distal of any food impaction or the like that is retrieved, enabling visual inspection of the anatomy post-retrieval without yet pulling out and re-inserting the scope N. In the same way, an irrigation channel may be extended distally of the scope N by effectively connecting such scope channel with the distal end 24 of the device 20 as by forming a conduit that is in fluid communication therebetween when the scope N is inserted in the device 20. Similarly, a tool channel of the scope may be extended within the wall 40 of the device body 30 so as to effectively enable relatively more distal deployment of any such tool, again, even if primary lumen 50 of the device 20 has already had suction applied and material drawn therein. These and other optional configurations and uses are contemplated as being within the spirit and scope of the present invention.

As shown in FIGS. 1 and 2, at the distal end 24 of the endoscope device 20, or its leading edge or rim, a nose 46 is formed substantially about the distal opening 34. The nose 46 may be rounded or tapered, as may be the body wall 40 or the outer surface 42 approaching or up to the distal end 24 and distal opening 34, in order to aid in smoothly intubating or passing the device 20 and scope N into the anatomy, more about which is said below in connection with FIGS. 7A-7C. In the exemplary embodiment, the distance from the step 36 to the nose 46 defines a length of the primary lumen 50 and the longest distance across the inner surface 44 defines a width of the primary lumen 50, such that in one variation the length is approximately twice the width; in other variations, the length may be greater than the width or less than the width. To illustrate, the endoscope device 20 may be formed wherein the distance from the step 36 to the nose 46 defines a length of between 15 mm and 80 mm and the longest distance across the inner surface 44 defines a width of between 12 mm and 40 mm, such that the length and the width define a volume within the primary lumen 50 of between approximately 1,700 mm³ and approximately 100,000 mm³. The secondary lumen 60, being configured in some embodiments as in FIGS. 1 and 2 for a net- or friction-fit on the distal end of an endoscope N and in others to allow a slip fit of the endoscope N within the secondary lumen 60, may be substantially annular with an inside diameter between 4 mm and 30 mm, as dictated by the intended use, and with a length of between 8 mm and 60 mm. It will be appreciated that the overall size of the exemplary endoscope device 20 may be as small as approximately 25 mm long with a diameter of approximately 12 mm and as large as up to approximately 100-125 mm long with a diameter of approximately 50 mm, though it will be appreciated by those skilled in the art that a variety of other sizes and size ratios of the various features are possible without departing from the spirit and scope of the invention.

Dimensionally, then, at the distal end 24 of the endoscope device 20 at which the primary lumen 50 is formed contiguous with the distal opening 34, in aspects of this embodiment, the length of the primary lumen 50 is, e.g., about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, or about 80 mm. In other aspects of this embodiment, in an endoscope device 20 disclosed herein the length of the primary lumen 50 is, e.g., at least 15 mm, at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, or at least 80 mm. In other aspects of this embodiment, in an endoscope device 20 disclosed herein the length of the primary lumen 50 is, e.g., at most 15 mm, at most 20 mm, at most 25 mm, at most 30 mm, at most 35 mm, at most 40 mm, at most 45 mm, at most 50 mm, at most 55 mm, at most 60 mm, at most 65 mm, at most 70 mm, at most 75 mm, or at most 80 mm.

In other aspects of this embodiment, in an endoscope device 20 disclosed herein the length of the primary lumen 50 is, e.g., about 15 mm to about 20 mm, about 15 mm to about 25 mm, about 15 mm to about 30 mm, about 15 mm to about 35 mm, about 15 mm to about 40 mm, about 15 mm to about 45 mm, about 15 mm to about 50 mm, about 15 mm to about 55 mm, about 15 mm to about 60 mm, about 15 mm to about 65 mm, about 15 mm to about 70 mm, about 15 mm to about 75 mm, about 15 mm to about 80 mm, about 20 mm to about 25 mm, about 20 mm to about 30 mm, about 20 mm to about 35 mm, about 20 mm to about 40 mm, about 20 mm to about 45 mm, about 20 mm to about 50 mm, about 20 mm to about 55 mm, about 20 mm to about 60 mm, about 20 mm to about 65 mm, about 20 mm to about 70 mm, about 20 mm to about 75 mm, about 20 mm to about 80 mm, about 25 mm to about 30 mm, about 25 mm to about 35 mm, about 25 mm to about 40 mm, about 25 mm to about 45 mm, about 25 mm to about 50 mm, about 25 mm to about 55 mm, about 25 mm to about 60 mm, about 25 mm to about 65 mm, about 25 mm to about 70 mm, about 25 mm to about 75 mm, about 25 mm to about 80 mm, about 30 mm to about 35 mm, about 30 mm to about 40 mm, about 30 mm to about 45 mm, about 30 mm to about 50 mm, about 30 mm to about 55 mm, about 30 mm to about 60 mm, about 30 mm to about 65 mm, about 30 mm to about 70 mm, about 30 mm to about 75 mm, about 30 mm to about 80 mm, about 35 mm to about 40 mm, about 35 mm to about 45 mm, about 35 mm to about 50 mm, about 35 mm to about 55 mm, about 35 mm to about 60 mm, about 35 mm to about 65 mm, about 35 mm to about 70 mm, about 35 mm to about 75 mm, about 35 mm to about 80 mm, about 40 mm to about 45 mm, about 40 mm to about 50 mm, about 40 mm to about 55 mm, about 40 mm to about 60 mm, about 40 mm to about 65 mm, about 40 mm to about 70 mm, about 40 mm to about 75 mm, about 40 mm to about 80 mm, about 45 mm to about 50 mm, about 45 mm to about 55 mm, about 45 mm to about 60 mm, about 45 mm to about 65 mm, about 45 mm to about 70 mm, about 45 mm to about 75 mm, about 45 mm to about 80 mm, about 50 mm to about 55 mm, about 50 mm to about 60 mm, about 50 mm to about 65 mm, about 50 mm to about 70 mm, about 50 mm to about 75 mm, about 50 mm to about 80 mm, about 55 mm to about 60 mm, about 55 mm to about 65 mm, about 55 mm to about 70 mm, about 55 mm to about 75 mm, about 55 mm to about 80 mm, about 60 mm to about 65 mm, about 60 mm to about 70 mm, about 60 mm to about 75 mm, about 60 mm to about 80 mm, about 65 mm to about 70 mm, about 65 mm to about 75 mm, about 65 mm to about 80 mm, about 70 mm to about 75 mm, about 70 mm to about 80 mm, or about 75 mm to about 80 mm.

In further aspects of this embodiment, the width of the primary lumen 50 is, e.g., about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, or about 40 mm. In still further aspects of this embodiment, the width of the primary lumen 50 is, e.g., at least 12 mm, at least 13 mm, at least 14 mm, at least 15 mm, at least 16 mm, at least 17 mm, at least 18 mm, at least 19 mm, at least 20 mm, at least 21 mm, at least 22 mm, at least 23 mm, at least 24 mm, at least 25 mm, at least 26 mm, at least 27 mm, at least 28 mm, at least 29 mm, at least 30 mm, at least 31 mm, at least 32 mm, at least 33 mm, at least 34 mm, at least 35 mm, at least 36 mm, at least 37 mm, at least 38 mm, at least 39 mm, or at least 40 mm. In still further aspects of this embodiment, the width of the primary lumen 50 is, e.g., at most 12 mm, at most 13 mm, at most 14 mm, at most 15 mm, at most 16 mm, at most 17 mm, at most 18 mm, at most 19 mm, at most 20 mm, at most 21 mm, at most 22 mm, at most 23 mm, at most 24 mm, at most 25 mm, at most 26 mm, at most 27 mm, at most 28 mm, at most 29 mm, at most 30 mm, at most 31 mm, at most 32 mm, at most 33 mm, at most 34 mm, at most 35 mm, at most 36 mm, at most 37 mm, at most 38 mm, at most 39 mm, or at most 40 mm.

In other aspects of this embodiment, in an endoscope device 20 disclosed herein the width of the primary lumen 50 is, e.g., about 12 mm to about 13 mm, about 12 mm to about 14 mm, about 12 mm to about 15 mm, about 12 mm to about 16 mm, about 12 mm to about 17 mm, about 12 mm to about 18 mm, about 12 mm to about 19 mm, about 12 mm to about 20 mm, about 12 mm to about 21 mm, about 12 mm to about 22 mm, about 12 mm to about 23 mm, about 12 mm to about 24 mm, about 12 mm to about 25 mm, about 12 mm to about 26 mm, about 12 mm to about 27 mm, about 12 mm to about 28 mm, about 12 mm to about 29 mm, about 12 mm to about 30 mm, about 12 mm to about 31 mm, about 12 mm to about 32 mm, about 12 mm to about 33 mm, about 12 mm to about 34 mm, about 12 mm to about 35 mm, about 12 mm to about 36 mm, about 12 mm to about 37 mm, about 12 mm to about 38 mm, about 12 mm to about 39 mm, about 12 mm to about 40 mm, about 13 mm to about 14 mm, about 13 mm to about 15 mm, about 13 mm to about 16 mm, about 13 mm to about 17 mm, about 13 mm to about 18 mm, about 13 mm to about 19 mm, about 13 mm to about 20 mm, about 13 mm to about 21 mm, about 13 mm to about 22 mm, about 13 mm to about 23 mm, about 13 mm to about 24 mm, about 13 mm to about 25 mm, about 13 mm to about 26 mm, about 13 mm to about 27 mm, about 13 mm to about 28 mm, about 13 mm to about 29 mm, about 13 mm to about 30 mm, about 13 mm to about 31 mm, about 13 mm to about 32 mm, about 13 mm to about 33 mm, about 13 mm to about 34 mm, about 13 mm to about 35 mm, about 13 mm to about 36 mm, about 13 mm to about 37 mm, about 13 mm to about 38 mm, about 13 mm to about 39 mm, about 13 mm to about 40 mm, about 14 mm to about 15 mm, about 14 mm to about 16 mm, about 14 mm to about 17 mm, about 14 mm to about 18 mm, about 14 mm to about 19 mm, about 14 mm to about 20 mm, about 14 mm to about 21 mm, about 14 mm to about 22 mm, about 14 mm to about 23 mm, about 14 mm to about 24 mm, about 14 mm to about 25 mm, about 14 mm to about 26 mm, about 14 mm to about 27 mm, about 14 mm to about 28 mm, about 14 mm to about 29 mm, about 14 mm to about 30 mm, about 14 mm to about 31 mm, about 14 mm to about 32 mm, about 14 mm to about 33 mm, about 14 mm to about 34 mm, about 14 mm to about 35 mm, about 14 mm to about 36 mm, about 14 mm to about 37 mm, about 14 mm to about 38 mm, about 14 mm to about 39 mm, about 14 mm to about 40 mm, about 15 mm to about 16 mm, about 15 mm to about 17 mm, about 15 mm to about 18 mm, about 15 mm to about 19 mm, about 15 mm to about 20 mm, about 15 mm to about 21 mm, about 15 mm to about 22 mm, about 15 mm to about 23 mm, about 15 mm to about 24 mm, about 15 mm to about 25 mm, about 15 mm to about 26 mm, about 15 mm to about 27 mm, about 15 mm to about 28 mm, about 15 mm to about 29 mm, about 15 mm to about 30 mm, about 15 mm to about 31 mm, about 15 mm to about 32 mm, about 15 mm to about 33 mm, about 15 mm to about 34 mm, about 15 mm to about 35 mm, about 15 mm to about 36 mm, about 15 mm to about 37 mm, about 15 mm to about 38 mm, about 15 mm to about 39 mm, about 15 mm to about 40 mm, about 16 mm to about 17 mm, about 16 mm to about 18 mm, about 16 mm to about 19 mm, about 16 mm to about 20 mm, about 16 mm to about 21 mm, about 16 mm to about 22 mm, about 16 mm to about 23 mm, about 16 mm to about 24 mm, about 16 mm to about 25 mm, about 16 mm to about 26 mm, about 16 mm to about 27 mm, about 16 mm to about 28 mm, about 16 mm to about 29 mm, about 16 mm to about 30 mm, about 16 mm to about 31 mm, about 16 mm to about 32 mm, about 16 mm to about 33 mm, about 16 mm to about 34 mm, about 16 mm to about 35 mm, about 16 mm to about 36 mm, about 16 mm to about 37 mm, about 16 mm to about 38 mm, about 16 mm to about 39 mm, about 16 mm to about 40 mm, about 17 mm to about 18 mm, about 17 mm to about 19 mm, about 17 mm to about 20 mm, about 17 mm to about 21 mm, about 17 mm to about 22 mm, about 17 mm to about 23 mm, about 17 mm to about 24 mm, about 17 mm to about 25 mm, about 17 mm to about 26 mm, about 17 mm to about 27 mm, about 17 mm to about 28 mm, about 17 mm to about 29 mm, about 17 mm to about 30 mm, about 17 mm to about 31 mm, about 17 mm to about 32 mm, about 17 mm to about 33 mm, about 17 mm to about 34 mm, about 17 mm to about 35 mm, about 17 mm to about 36 mm, about 17 mm to about 37 mm, about 17 mm to about 38 mm, about 17 mm to about 39 mm, about 17 mm to about 40 mm, about 18 mm to about 19 mm, about 18 mm to about 20 mm, about 18 mm to about 21 mm, about 18 mm to about 22 mm, about 18 mm to about 23 mm, about 18 mm to about 24 mm, about 18 mm to about 25 mm, about 18 mm to about 26 mm, about 18 mm to about 27 mm, about 18 mm to about 28 mm, about 18 mm to about 29 mm, about 18 mm to about 30 mm, about 18 mm to about 31 mm, about 18 mm to about 32 mm, about 18 mm to about 33 mm, about 18 mm to about 34 mm, about 18 mm to about 35 mm, about 18 mm to about 36 mm, about 18 mm to about 37 mm, about 18 mm to about 38 mm, about 18 mm to about 39 mm, about 18 mm to about 40 mm, about 19 mm to about 20 mm, about 19 mm to about 21 mm, about 19 mm to about 22 mm, about 19 mm to about 23 mm, about 19 mm to about 24 mm, about 19 mm to about 25 mm, about 19 mm to about 26 mm, about 19 mm to about 27 mm, about 19 mm to about 28 mm, about 19 mm to about 29 mm, about 19 mm to about 30 mm, about 19 mm to about 31 mm, about 19 mm to about 32 mm, about 19 mm to about 33 mm, about 19 mm to about 34 mm, about 19 mm to about 35 mm, about 19 mm to about 36 mm, about 19 mm to about 37 mm, about 19 mm to about 38 mm, about 19 mm to about 39 mm, about 19 mm to about 40 mm, about 20 mm to about 21 mm, about 20 mm to about 22 mm, about 20 mm to about 23 mm, about 20 mm to about 24 mm, about 20 mm to about 25 mm, about 20 mm to about 26 mm, about 20 mm to about 27 mm, about 20 mm to about 28 mm, about 20 mm to about 29 mm, about 20 mm to about 30 mm, about 20 mm to about 31 mm, about 20 mm to about 32 mm, about 20 mm to about 33 mm, about 20 mm to about 34 mm, about 20 mm to about 35 mm, about 20 mm to about 36 mm, about 20 mm to about 37 mm, about 20 mm to about 38 mm, about 20 mm to about 39 mm, about 20 mm to about 40 mm, about 21 mm to about 22 mm, about 21 mm to about 23 mm, about 21 mm to about 24 mm, about 21 mm to about 25 mm, about 21 mm to about 26 mm, about 21 mm to about 27 mm, about 21 mm to about 28 mm, about 21 mm to about 29 mm, about 21 mm to about 30 mm, about 21 mm to about 31 mm, about 21 mm to about 32 mm, about 21 mm to about 33 mm, about 21 mm to about 34 mm, about 21 mm to about 35 mm, about 21 mm to about 36 mm, about 21 mm to about 37 mm, about 21 mm to about 38 mm, about 21 mm to about 39 mm, about 21 mm to about 40 mm, about 22 mm to about 23 mm, about 22 mm to about 24 mm, about 22 mm to about 25 mm, about 22 mm to about 26 mm, about 22 mm to about 27 mm, about 22 mm to about 28 mm, about 22 mm to about 29 mm, about 22 mm to about 30 mm, about 22 mm to about 31 mm, about 22 mm to about 32 mm, about 22 mm to about 33 mm, about 22 mm to about 34 mm, about 22 mm to about 35 mm, about 22 mm to about 36 mm, about 22 mm to about 37 mm, about 22 mm to about 38 mm, about 22 mm to about 39 mm, about 22 mm to about 40 mm, about 23 mm to about 24 mm, about 23 mm to about 25 mm, about 23 mm to about 26 mm, about 23 mm to about 27 mm, about 23 mm to about 28 mm, about 23 mm to about 29 mm, about 23 mm to about 30 mm, about 23 mm to about 31 mm, about 23 mm to about 32 mm, about 23 mm to about 33 mm, about 23 mm to about 34 mm, about 23 mm to about 35 mm, about 23 mm to about 36 mm, about 23 mm to about 37 mm, about 23 mm to about 38 mm, about 23 mm to about 39 mm, about 23 mm to about 40 mm, about 24 mm to about 25 mm, about 24 mm to about 26 mm, about 24 mm to about 27 mm, about 24 mm to about 28 mm, about 24 mm to about 29 mm, about 24 mm to about 30 mm, about 24 mm to about 31 mm, about 24 mm to about 32 mm, about 24 mm to about 33 mm, about 24 mm to about 34 mm, about 24 mm to about 35 mm, about 24 mm to about 36 mm, about 24 mm to about 37 mm, about 24 mm to about 38 mm, about 24 mm to about 39 mm, about 24 mm to about 40 mm, about 25 mm to about 26 mm, about 25 mm to about 27 mm, about 25 mm to about 28 mm, about 25 mm to about 29 mm, about 25 mm to about 30 mm, about 25 mm to about 31 mm, about 25 mm to about 32 mm, about 25 mm to about 33 mm, about 25 mm to about 34 mm, about 25 mm to about 35 mm, about 25 mm to about 36 mm, about 25 mm to about 37 mm, about 25 mm to about 38 mm, about 25 mm to about 39 mm, about 25 mm to about 40 mm, about 26 mm to about 27 mm, about 26 mm to about 28 mm, about 26 mm to about 29 mm, about 26 mm to about 30 mm, about 26 mm to about 31 mm, about 26 mm to about 32 mm, about 26 mm to about 33 mm, about 26 mm to about 34 mm, about 26 mm to about 35 mm, about 26 mm to about 36 mm, about 26 mm to about 37 mm, about 26 mm to about 38 mm, about 26 mm to about 39 mm, about 26 mm to about 40 mm, about 27 mm to about 28 mm, about 27 mm to about 29 mm, about 27 mm to about 30 mm, about 27 mm to about 31 mm, about 27 mm to about 32 mm, about 27 mm to about 33 mm, about 27 mm to about 34 mm, about 27 mm to about 35 mm, about 27 mm to about 36 mm, about 27 mm to about 37 mm, about 27 mm to about 38 mm, about 27 mm to about 39 mm, about 27 mm to about 40 mm, about 28 mm to about 29 mm, about 28 mm to about 30 mm, about 28 mm to about 31 mm, about 28 mm to about 32 mm, about 28 mm to about 33 mm, about 28 mm to about 34 mm, about 28 mm to about 35 mm, about 28 mm to about 36 mm, about 28 mm to about 37 mm, about 28 mm to about 38 mm, about 28 mm to about 39 mm, about 28 mm to about 40 mm, about 29 mm to about 30 mm, about 29 mm to about 31 mm, about 29 mm to about 32 mm, about 29 mm to about 33 mm, about 29 mm to about 34 mm, about 29 mm to about 35 mm, about 29 mm to about 36 mm, about 29 mm to about 37 mm, about 29 mm to about 38 mm, about 29 mm to about 39 mm, about 29 mm to about 40 mm, about 30 mm to about 31 mm, about 30 mm to about 32 mm, about 30 mm to about 33 mm, about 30 mm to about 34 mm, about 30 mm to about 35 mm, about 30 mm to about 36 mm, about 30 mm to about 37 mm, about 30 mm to about 38 mm, about 30 mm to about 39 mm, about 30 mm to about 40 mm, about 31 mm to about 32 mm, about 31 mm to about 33 mm, about 31 mm to about 34 mm, about 31 mm to about 35 mm, about 31 mm to about 36 mm, about 31 mm to about 37 mm, about 31 mm to about 38 mm, about 31 mm to about 39 mm, about 31 mm to about 40 mm, about 32 mm to about 33 mm, about 32 mm to about 34 mm, about 32 mm to about 35 mm, about 32 mm to about 36 mm, about 32 mm to about 37 mm, about 32 mm to about 38 mm, about 32 mm to about 39 mm, about 32 mm to about 40 mm, about 33 mm to about 34 mm, about 33 mm to about 35 mm, about 33 mm to about 36 mm, about 33 mm to about 37 mm, about 33 mm to about 38 mm, about 33 mm to about 39 mm, about 33 mm to about 40 mm, about 34 mm to about 35 mm, about 34 mm to about 36 mm, about 34 mm to about 37 mm, about 34 mm to about 38 mm, about 34 mm to about 39 mm, about 34 mm to about 40 mm, about 35 mm to about 36 mm, about 35 mm to about 37 mm, about 35 mm to about 38 mm, about 35 mm to about 39 mm, about 35 mm to about 40 mm, about 36 mm to about 37 mm, about 36 mm to about 38 mm, about 36 mm to about 39 mm, about 36 mm to about 40 mm, about 37 mm to about 38 mm, about 37 mm to about 39 mm, about 37 mm to about 40 mm, about 38 mm to about 39 mm, about 38 mm to about 40 mm, or about 39 mm to about 40 mm.

In further aspects of this embodiment, the volume of the primary lumen 50 is, e.g., about 1,700 mm³, about 1,800 mm³, about 1,900 mm³, about 2,000 mm³, about 2,200 mm³, about 2,400 mm³, about 2,600 mm³, about 2,800 mm³, about 3,000 mm³, about 4,000 mm³, about 5,000 mm³, about 6,000 mm³, about 7,000 mm³, about 8,000 mm³, about 9,000 mm³, about 10,000 mm³, about 12,000 mm³, about 14,000 mm³, about 16,000 mm³, about 18,000 mm³, about 20,000 mm³, about 25,000 mm³, about 30,000 mm³, about 35,000 mm³, about 40,000 mm³, about 45,000 mm³, about 50,000 mm³, about 60,000 mm³, about 70,000 mm³, about 80,000 mm³, about 90,000 mm³, or about 100,000 mm³. In still further aspects of this embodiment, the volume of the primary lumen 50 is, e.g., at least 1,700 mm³, at least 1,800 mm³, at least 1,900 mm³, at least 2,000 mm³, at least 2,200 mm³, at least 2,400 mm³, at least 2,600 mm³, at least 2,800 mm³, at least 3,000 mm³, at least 4,000 mm³, at least 5,000 mm³, at least 6,000 mm³, at least 7,000 mm³, at least 8,000 mm³, at least 9,000 mm³, at least 10,000 mm³, at least 12,000 mm³, at least 14,000 mm³, at least 16,000 mm³, at least 18,000 mm³, at least 20,000 mm³, at least 25,000 mm³, at least 30,000 mm³, at least 35,000 mm³, at least 40,000 mm³, at least 45,000 mm³, at least 50,000 mm³, at least 60,000 mm³, at least 70,000 mm³, at least 80,000 mm³, at least 90,000 mm³, or at least 100,000 mm³. In still further aspects of this embodiment, the volume of the primary lumen 50 is, e.g., at most 1,700 mm³, at most 1,800 mm³, at most 1,900 mm³, at most 2,000 mm³, at most 2,200 mm³, at most 2,400 mm³, at most 2,600 mm³, at most 2,800 mm³, at most 3,000 mm³, at most 4,000 mm³, at most 5,000 mm³, at most 6,000 mm³, at most 7,000 mm³, at most 8,000 mm³, at most 9,000 mm³, at most 10,000 mm³, at most 12,000 mm³, at most 14,000 mm³, at most 16,000 mm³, at most 18,000 mm³, at most 20,000 mm³, at most 25,000 mm³, at most 30,000 mm³, at most 35,000 mm³, at most 40,000 mm³, at most 45,000 mm³, at most 50,000 mm³, at most 60,000 mm³, at most 70,000 mm³, at most 80,000 mm³, at most 90,000 mm³, or at most 100,000 mm³.

In other aspects of this embodiment, in an endoscope device 20 disclosed herein the volume of the primary lumen 50 is, e.g., about 1,700 mm³ to about 1,800 mm³, about 1,700 mm³ to about 1,900 mm³, about 1,700 mm³ to about 2,000 mm³, about 1,700 mm³ to about 2,200 mm³, about 1,700 mm³ to about 2,400 mm³, about 1,700 mm³ to about 2,600 mm³, about 1,700 mm³ to about 2,800 mm³, about 1,700 mm³ to about 3,000 mm³, about 1,700 mm³ to about 4,000 mm³, about 1,700 mm³ to about 5,000 mm³, about 1,700 mm³ to about 6,000 mm³, about 1,700 mm³ to about 7,000 mm³, about 1,700 mm³ to about 8,000 mm³, about 1,700 mm³ to about 9,000 mm³, about 1,700 mm³ to about 10,000 mm³, about 1,700 mm³ to about 12,000 mm³, about 1,700 mm³ to about 14,000 mm³, about 1,700 mm³ to about 16,000 mm³, about 1,700 mm³ to about 18,000 mm³, about 1,700 mm³ to about 20,000 mm³, about 1,700 mm³ to about 25,000 mm³, about 1,700 mm³ to about 30,000 mm³, about 1,700 mm³ to about 35,000 mm³, about 1,700 mm³ to about 40,000 mm³, about 1,700 mm³ to about 45,000 mm³, about 1,700 mm³ to about 50,000 mm³, about 1,700 mm³ to about 60,000 mm³, about 1,700 mm³ to about 70,000 mm³, about 1,700 mm³ to about 80,000 mm³, about 1,700 mm³ to about 90,000 mm³, about 1,700 mm³ to about 100,000 mm³, about 1,800 mm³ to about 1,900 mm³, about 1,800 mm³ to about 2,000 mm³, about 1,800 mm³ to about 2,200 mm³, about 1,800 mm³ to about 2,400 mm³, about 1,800 mm³ to about 2,600 mm³, about 1,800 mm³ to about 2,800 mm³, about 1,800 mm³ to about 3,000 mm³, about 1,800 mm³ to about 4,000 mm³, about 1,800 mm³ to about 5,000 mm³, about 1,800 mm³ to about 6,000 mm³, about 1,800 mm³ to about 7,000 mm³, about 1,800 mm³ to about 8,000 mm³, about 1,800 mm³ to about 9,000 mm³, about 1,800 mm³ to about 10,000 mm³, about 1,800 mm³ to about 12,000 mm³, about 1,800 mm³ to about 14,000 mm³, about 1,800 mm³ to about 16,000 mm³, about 1,800 mm³ to about 18,000 mm³, about 1,800 mm³ to about 20,000 mm³, about 1,800 mm³ to about 25,000 mm³, about 1,800 mm³ to about 30,000 mm³, about 1,800 mm³ to about 35,000 mm³, about 1,800 mm³ to about 40,000 mm³, about 1,800 mm³ to about 45,000 mm³, about 1,800 mm³ to about 50,000 mm³, about 1,800 mm³ to about 60,000 mm³, about 1,800 mm³ to about 70,000 mm³, about 1,800 mm³ to about 80,000 mm³, about 1,800 mm³ to about 90,000 mm³, about 1,800 mm³ to about 100,000 mm³, about 1,900 mm³ to about 2,000 mm³, about 1,900 mm³ to about 2,200 mm³, about 1,900 mm³ to about 2,400 mm³, about 1,900 mm³ to about 2,600 mm³, about 1,900 mm³ to about 2,800 mm³, about 1,900 mm³ to about 3,000 mm³, about 1,900 mm³ to about 4,000 mm³, about 1,900 mm³ to about 5,000 mm³, about 1,900 mm³ to about 6,000 mm³, about 1,900 mm³ to about 7,000 mm³, about 1,900 mm³ to about 8,000 mm³, about 1,900 mm³ to about 9,000 mm³, about 1,900 mm³ to about 10,000 mm³, about 1,900 mm³ to about 12,000 mm³, about 1,900 mm³ to about 14,000 mm³, about 1,900 mm³ to about 16,000 mm³, about 1,900 mm³ to about 18,000 mm³, about 1,900 mm³ to about 20,000 mm³, about 1,900 mm³ to about 25,000 mm³, about 1,900 mm³ to about 30,000 mm³, about 1,900 mm³ to about 35,000 mm³, about 1,900 mm³ to about 40,000 mm³, about 1,900 mm³ to about 45,000 mm³, about 1,900 mm³ to about 50,000 mm³, about 1,900 mm³ to about 60,000 mm³, about 1,900 mm³ to about 70,000 mm³, about 1,900 mm³ to about 80,000 mm³, about 1,900 mm³ to about 90,000 mm³, about 1,900 mm³ to about 100,000 mm³, about 2,000 mm³ to about 2,200 mm³, about 2,000 mm³ to about 2,400 mm³, about 2,000 mm³ to about 2,600 mm³, about 2,000 mm³ to about 2,800 mm³, about 2,000 mm³ to about 3,000 mm³, about 2,000 mm³ to about 4,000 mm³, about 2,000 mm³ to about 5,000 mm³, about 2,000 mm³ to about 6,000 mm³, about 2,000 mm³ to about 7,000 mm³, about 2,000 mm³ to about 8,000 mm³, about 2,000 mm³ to about 9,000 mm³, about 2,000 mm³ to about 10,000 mm³, about 2,000 mm³ to about 12,000 mm³, about 2,000 mm³ to about 14,000 mm³, about 2,000 mm³ to about 16,000 mm³, about 2,000 mm³ to about 18,000 mm³, about 2,000 mm³ to about 20,000 mm³, about 2,000 mm³ to about 25,000 mm³, about 2,000 mm³ to about 30,000 mm³, about 2,000 mm³ to about 35,000 mm³, about 2,000 mm³ to about 40,000 mm³, about 2,000 mm³ to about 45,000 mm³, about 2,000 mm³ to about 50,000 mm³, about 2,000 mm³ to about 60,000 mm³, about 2,000 mm³ to about 70,000 mm³, about 2,000 mm³ to about 80,000 mm³, about 2,000 mm³ to about 90,000 mm³, about 2,000 mm³ to about 100,000 mm³, about 2,200 mm³ to about 2,400 mm³, about 2,200 mm³ to about 2,600 mm³, about 2,200 mm³ to about 2,800 mm³, about 2,200 mm³ to about 3,000 mm³, about 2,200 mm³ to about 4,000 mm³, about 2,200 mm³ to about 5,000 mm³, about 2,200 mm³ to about 6,000 mm³, about 2,200 mm³ to about 7,000 mm³, about 2,200 mm³ to about 8,000 mm³, about 2,200 mm³ to about 9,000 mm³, about 2,200 mm³ to about 10,000 mm³, about 2,200 mm³ to about 12,000 mm³, about 2,200 mm³ to about 14,000 mm³, about 2,200 mm³ to about 16,000 mm³, about 2,200 mm³ to about 18,000 mm³, about 2,200 mm³ to about 20,000 mm³, about 2,200 mm³ to about 25,000 mm³, about 2,200 mm³ to about 30,000 mm³, about 2,200 mm³ to about 35,000 mm³, about 2,200 mm³ to about 40,000 mm³, about 2,200 mm³ to about 45,000 mm³, about 2,200 mm³ to about 50,000 mm³, about 2,200 mm³ to about 60,000 mm³, about 2,200 mm³ to about 70,000 mm³, about 2,200 mm³ to about 80,000 mm³, about 2,200 mm³ to about 90,000 mm³, about 2,200 mm³ to about 100,000 mm³, about 2,400 mm³ to about 2,600 mm³, about 2,400 mm³ to about 2,800 mm³, about 2,400 mm³ to about 3,000 mm³, about 2,400 mm³ to about 4,000 mm³, about 2,400 mm³ to about 5,000 mm³, about 2,400 mm³ to about 6,000 mm³, about 2,400 mm³ to about 7,000 mm³, about 2,400 mm³ to about 8,000 mm³, about 2,400 mm³ to about 9,000 mm³, about 2,400 mm³ to about 10,000 mm³, about 2,400 mm³ to about 12,000 mm³, about 2,400 mm³ to about 14,000 mm³, about 2,400 mm³ to about 16,000 mm³, about 2,400 mm³ to about 18,000 mm³, about 2,400 mm³ to about 20,000 mm³, about 2,400 mm³ to about 25,000 mm³, about 2,400 mm³ to about 30,000 mm³, about 2,400 mm³ to about 35,000 mm³, about 2,400 mm³ to about 40,000 mm³, about 2,400 mm³ to about 45,000 mm³, about 2,400 mm³ to about 50,000 mm³, about 2,400 mm³ to about 60,000 mm³, about 2,400 mm³ to about 70,000 mm³, about 2,400 mm³ to about 80,000 mm³, about 2,400 mm³ to about 90,000 mm³, about 2,400 mm³ to about 100,000 mm³, about 2,600 mm³ to about 2,800 mm³, about 2,600 mm³ to about 3,000 mm³, about 2,600 mm³ to about 4,000 mm³, about 2,600 mm³ to about 5,000 mm³, about 2,600 mm³ to about 6,000 mm³, about 2,600 mm³ to about 7,000 mm³, about 2,600 mm³ to about 8,000 mm³, about 2,600 mm³ to about 9,000 mm³, about 2,600 mm³ to about 10,000 mm³, about 2,600 mm³ to about 12,000 mm³, about 2,600 mm³ to about 14,000 mm³, about 2,600 mm³ to about 16,000 mm³, about 2,600 mm³ to about 18,000 mm³, about 2,600 mm³ to about 20,000 mm³, about 2,600 mm³ to about 25,000 mm³, about 2,600 mm³ to about 30,000 mm³, about 2,600 mm³ to about 35,000 mm³, about 2,600 mm³ to about 40,000 mm³, about 2,600 mm³ to about 45,000 mm³, about 2,600 mm³ to about 50,000 mm³, about 2,600 mm³ to about 60,000 mm³, about 2,600 mm³ to about 70,000 mm³, about 2,600 mm³ to about 80,000 mm³, about 2,600 mm³ to about 90,000 mm³, about 2,600 mm³ to about 100,000 mm³, about 2,800 mm³ to about 3,000 mm³, about 2,800 mm³ to about 4,000 mm³, about 2,800 mm³ to about 5,000 mm³, about 2,800 mm³ to about 6,000 mm³, about 2,800 mm³ to about 7,000 mm³, about 2,800 mm³ to about 8,000 mm³, about 2,800 mm³ to about 9,000 mm³, about 2,800 mm³ to about 10,000 mm³, about 2,800 mm³ to about 12,000 mm³, about 2,800 mm³ to about 14,000 mm³, about 2,800 mm³ to about 16,000 mm³, about 2,800 mm³ to about 18,000 mm³, about 2,800 mm³ to about 20,000 mm³, about 2,800 mm³ to about 25,000 mm³, about 2,800 mm³ to about 30,000 mm³, about 2,800 mm³ to about 35,000 mm³, about 2,800 mm³ to about 40,000 mm³, about 2,800 mm³ to about 45,000 mm³, about 2,800 mm³ to about 50,000 mm³, about 2,800 mm³ to about 60,000 mm³, about 2,800 mm³ to about 70,000 mm³, about 2,800 mm³ to about 80,000 mm³, about 2,800 mm³ to about 90,000 mm³, about 2,800 mm³ to about 100,000 mm³, about 3,000 mm³ to about 4,000 mm³, about 3,000 mm³ to about 5,000 mm³, about 3,000 mm³ to about 6,000 mm³, about 3,000 mm³ to about 7,000 mm³, about 3,000 mm³ to about 8,000 mm³, about 3,000 mm³ to about 9,000 mm³, about 3,000 mm³ to about 10,000 mm³, about 3,000 mm³ to about 12,000 mm³, about 3,000 mm³ to about 14,000 mm³, about 3,000 mm³ to about 16,000 mm³, about 3,000 mm³ to about 18,000 mm³, about 3,000 mm³ to about 20,000 mm³, about 3,000 mm³ to about 25,000 mm³, about 3,000 mm³ to about 30,000 mm³, about 3,000 mm³ to about 35,000 mm³, about 3,000 mm³ to about 40,000 mm³, about 3,000 mm³ to about 45,000 mm³, about 3,000 mm³ to about 50,000 mm³, about 3,000 mm³ to about 60,000 mm³, about 3,000 mm³ to about 70,000 mm³, about 3,000 mm³ to about 80,000 mm³, about 3,000 mm³ to about 90,000 mm³, about 3,000 mm³ to about 100,000 mm³, about 4,000 mm³ to about 5,000 mm³, about 4,000 mm³ to about 6,000 mm³, about 4,000 mm³ to about 7,000 mm³, about 4,000 mm³ to about 8,000 mm³, about 4,000 mm³ to about 9,000 mm³, about 4,000 mm³ to about 10,000 mm³, about 4,000 mm³ to about 12,000 mm³, about 4,000 mm³ to about 14,000 mm³, about 4,000 mm³ to about 16,000 mm³, about 4,000 mm³ to about 18,000 mm³, about 4,000 mm³ to about 20,000 mm³, about 4,000 mm³ to about 25,000 mm³, about 4,000 mm³ to about 30,000 mm³, about 4,000 mm³ to about 35,000 mm³, about 4,000 mm³ to about 40,000 mm³, about 4,000 mm³ to about 45,000 mm³, about 4,000 mm³ to about 50,000 mm³, about 4,000 mm³ to about 60,000 mm³, about 4,000 mm³ to about 70,000 mm³, about 4,000 mm³ to about 80,000 mm³, about 4,000 mm³ to about 90,000 mm³, about 4,000 mm³ to about 100,000 mm³, about 5,000 mm³ to about 6,000 mm³, about 5,000 mm³ to about 7,000 mm³, about 5,000 mm³ to about 8,000 mm³, about 5,000 mm³ to about 9,000 mm³, about 5,000 mm³ to about 10,000 mm³, about 5,000 mm³ to about 12,000 mm³, about 5,000 mm³ to about 14,000 mm³, about 5,000 mm³ to about 16,000 mm³, about 5,000 mm³ to about 18,000 mm³, about 5,000 mm³ to about 20,000 mm³, about 5,000 mm³ to about 25,000 mm³, about 5,000 mm³ to about 30,000 mm³, about 5,000 mm³ to about 35,000 mm³, about 5,000 mm³ to about 40,000 mm³, about 5,000 mm³ to about 45,000 mm³, about 5,000 mm³ to about 50,000 mm³, about 5,000 mm³ to about 60,000 mm³, about 5,000 mm³ to about 70,000 mm³, about 5,000 mm³ to about 80,000 mm³, about 5,000 mm³ to about 90,000 mm³, about 5,000 mm³ to about 100,000 mm³, about 6,000 mm³ to about 7,000 mm³, about 6,000 mm³ to about 8,000 mm³, about 6,000 mm³ to about 9,000 mm³, about 6,000 mm³ to about 10,000 mm³, about 6,000 mm³ to about 12,000 mm³, about 6,000 mm³ to about 14,000 mm³, about 6,000 mm³ to about 16,000 mm³, about 6,000 mm³ to about 18,000 mm³, about 6,000 mm³ to about 20,000 mm³, about 6,000 mm³ to about 25,000 mm³, about 6,000 mm³ to about 30,000 mm³, about 6,000 mm³ to about 35,000 mm³, about 6,000 mm³ to about 40,000 mm³, about 6,000 mm³ to about 45,000 mm³, about 6,000 mm³ to about 50,000 mm³, about 6,000 mm³ to about 60,000 mm³, about 6,000 mm³ to about 70,000 mm³, about 6,000 mm³ to about 80,000 mm³, about 6,000 mm³ to about 90,000 mm³, about 6,000 mm³ to about 100,000 mm³, about 7,000 mm³ to about 8,000 mm³, about 7,000 mm³ to about 9,000 mm³, about 7,000 mm³ to about 10,000 mm³, about 7,000 mm³ to about 12,000 mm³, about 7,000 mm³ to about 14,000 mm³, about 7,000 mm³ to about 16,000 mm³, about 7,000 mm³ to about 18,000 mm³, about 7,000 mm³ to about 20,000 mm³, about 7,000 mm³ to about 25,000 mm³, about 7,000 mm³ to about 30,000 mm³, about 7,000 mm³ to about 35,000 mm³, about 7,000 mm³ to about 40,000 mm³, about 7,000 mm³ to about 45,000 mm³, about 7,000 mm³ to about 50,000 mm³, about 7,000 mm³ to about 60,000 mm³, about 7,000 mm³ to about 70,000 mm³, about 7,000 mm³ to about 80,000 mm³, about 7,000 mm³ to about 90,000 mm³, about 7,000 mm³ to about 100,000 mm³, about 8,000 mm³ to about 9,000 mm³, about 8,000 mm³ to about 10,000 mm³, about 8,000 mm³ to about 12,000 mm³, about 8,000 mm³ to about 14,000 mm³, about 8,000 mm³ to about 16,000 mm³, about 8,000 mm³ to about 18,000 mm³, about 8,000 mm³ to about 20,000 mm³, about 8,000 mm³ to about 25,000 mm³, about 8,000 mm³ to about 30,000 mm³, about 8,000 mm³ to about 35,000 mm³, about 8,000 mm³ to about 40,000 mm³, about 8,000 mm³ to about 45,000 mm³, about 8,000 mm³ to about 50,000 mm³, about 8,000 mm³ to about 60,000 mm³, about 8,000 mm³ to about 70,000 mm³, about 8,000 mm³ to about 80,000 mm³, about 8,000 mm³ to about 90,000 mm³, about 8,000 mm³ to about 100,000 mm³, about 9,000 mm³ to about 10,000 mm³, about 9,000 mm³ to about 12,000 mm³, about 9,000 mm³ to about 14,000 mm³, about 9,000 mm³ to about 16,000 mm³, about 9,000 mm³ to about 18,000 mm³, about 9,000 mm³ to about 20,000 mm³, about 9,000 mm³ to about 25,000 mm³, about 9,000 mm³ to about 30,000 mm³, about 9,000 mm³ to about 35,000 mm³, about 9,000 mm³ to about 40,000 mm³, about 9,000 mm³ to about 45,000 mm³, about 9,000 mm³ to about 50,000 mm³, about 9,000 mm³ to about 60,000 mm³, about 9,000 mm³ to about 70,000 mm³, about 9,000 mm³ to about 80,000 mm³, about 9,000 mm³ to about 90,000 mm³, about 9,000 mm³ to about 100,000 mm³, about 10,000 mm³ to about 12,000 mm³, about 10,000 mm³ to about 14,000 mm³, about 10,000 mm³ to about 16,000 mm³, about 10,000 mm³ to about 18,000 mm³, about 10,000 mm³ to about 20,000 mm³, about 10,000 mm³ to about 25,000 mm³, about 10,000 mm³ to about 30,000 mm³, about 10,000 mm³ to about 35,000 mm³, about 10,000 mm³ to about 40,000 mm³, about 10,000 mm³ to about 45,000 mm³, about 10,000 mm³ to about 50,000 mm³, about 10,000 mm³ to about 60,000 mm³, about 10,000 mm³ to about 70,000 mm³, about 10,000 mm³ to about 80,000 mm³, about 10,000 mm³ to about 90,000 mm³, about 10,000 mm³ to about 100,000 mm³, about 12,000 mm³ to about 14,000 mm³, about 12,000 mm³ to about 16,000 mm³, about 12,000 mm³ to about 18,000 mm³, about 12,000 mm³ to about 20,000 mm³, about 12,000 mm³ to about 25,000 mm³, about 12,000 mm³ to about 30,000 mm³, about 12,000 mm³ to about 35,000 mm³, about 12,000 mm³ to about 40,000 mm³, about 12,000 mm³ to about 45,000 mm³, about 12,000 mm³ to about 50,000 mm³, about 12,000 mm³ to about 60,000 mm³, about 12,000 mm³ to about 70,000 mm³, about 12,000 mm³ to about 80,000 mm³, about 12,000 mm³ to about 90,000 mm³, about 12,000 mm³ to about 100,000 mm³, about 14,000 mm³ to about 16,000 mm³, about 14,000 mm³ to about 18,000 mm³, about 14,000 mm³ to about 20,000 mm³, about 14,000 mm³ to about 25,000 mm³, about 14,000 mm³ to about 30,000 mm³, about 14,000 mm³ to about 35,000 mm³, about 14,000 mm³ to about 40,000 mm³, about 14,000 mm³ to about 45,000 mm³, about 14,000 mm³ to about 50,000 mm³, about 14,000 mm³ to about 60,000 mm³, about 14,000 mm³ to about 70,000 mm³, about 14,000 mm³ to about 80,000 mm³, about 14,000 mm³ to about 90,000 mm³, about 14,000 mm³ to about 100,000 mm³, about 16,000 mm³ to about 18,000 mm³, about 16,000 mm³ to about 20,000 mm³, about 16,000 mm³ to about 25,000 mm³, about 16,000 mm³ to about 30,000 mm³, about 16,000 mm³ to about 35,000 mm³, about 16,000 mm³ to about 40,000 mm³, about 16,000 mm³ to about 45,000 mm³, about 16,000 mm³ to about 50,000 mm³, about 16,000 mm³ to about 60,000 mm³, about 16,000 mm³ to about 70,000 mm³, about 16,000 mm³ to about 80,000 mm³, about 16,000 mm³ to about 90,000 mm³, about 16,000 mm³ to about 100,000 mm³, about 18,000 mm³ to about 20,000 mm³, about 18,000 mm³ to about 25,000 mm³, about 18,000 mm³ to about 30,000 mm³, about 18,000 mm³ to about 35,000 mm³, about 18,000 mm³ to about 40,000 mm³, about 18,000 mm³ to about 45,000 mm³, about 18,000 mm³ to about 50,000 mm³, about 18,000 mm³ to about 60,000 mm³, about 18,000 mm³ to about 70,000 mm³, about 18,000 mm³ to about 80,000 mm³, about 18,000 mm³ to about 90,000 mm³, about 18,000 mm³ to about 100,000 mm³, about 20,000 mm³ to about 25,000 mm³, about 20,000 mm³ to about 30,000 mm³, about 20,000 mm³ to about 35,000 mm³, about 20,000 mm³ to about 40,000 mm³, about 20,000 mm³ to about 45,000 mm³, about 20,000 mm³ to about 50,000 mm³, about 20,000 mm³ to about 60,000 mm³, about 20,000 mm³ to about 70,000 mm³, about 20,000 mm³ to about 80,000 mm³, about 20,000 mm³ to about 90,000 mm³, about 20,000 mm³ to about 100,000 mm³, about 25,000 mm³ to about 30,000 mm³, about 25,000 mm³ to about 35,000 mm³, about 25,000 mm³ to about 40,000 mm³, about 25,000 mm³ to about 45,000 mm³, about 25,000 mm³ to about 50,000 mm³, about 25,000 mm³ to about 60,000 mm³, about 25,000 mm³ to about 70,000 mm³, about 25,000 mm³ to about 80,000 mm³, about 25,000 mm³ to about 90,000 mm³, about 25,000 mm³ to about 100,000 mm³, about 30,000 mm³ to about 35,000 mm³, about 30,000 mm³ to about 40,000 mm³, about 30,000 mm³ to about 45,000 mm³, about 30,000 mm³ to about 50,000 mm³, about 30,000 mm³ to about 60,000 mm³, about 30,000 mm³ to about 70,000 mm³, about 30,000 mm³ to about 80,000 mm³, about 30,000 mm³ to about 90,000 mm³, about 30,000 mm³ to about 100,000 mm³, about 35,000 mm³ to about 40,000 mm³, about 35,000 mm³ to about 45,000 mm³, about 35,000 mm³ to about 50,000 mm³, about 35,000 mm³ to about 60,000 mm³, about 35,000 mm³ to about 70,000 mm³, about 35,000 mm³ to about 80,000 mm³, about 35,000 mm³ to about 90,000 mm³, about 35,000 mm³ to about 100,000 mm³, about 40,000 mm³ to about 45,000 mm³, about 40,000 mm³ to about 50,000 mm³, about 40,000 mm³ to about 60,000 mm³, about 40,000 mm³ to about 70,000 mm³, about 40,000 mm³ to about 80,000 mm³, about 40,000 mm³ to about 90,000 mm³, about 40,000 mm³ to about 100,000 mm³, about 45,000 mm³ to about 50,000 mm³, about 45,000 mm³ to about 60,000 mm³, about 45,000 mm³ to about 70,000 mm³, about 45,000 mm³ to about 80,000 mm³, about 45,000 mm³ to about 90,000 mm³, about 45,000 mm³ to about 100,000 mm³, about 50,000 mm³ to about 60,000 mm³, about 50,000 mm³ to about 70,000 mm³, about 50,000 mm³ to about 80,000 mm³, about 50,000 mm³ to about 90,000 mm³, about 50,000 mm³ to about 100,000 mm³, about 60,000 mm³ to about 70,000 mm³, about 60,000 mm³ to about 80,000 mm³, about 60,000 mm³ to about 90,000 mm³, about 60,000 mm³ to about 100,000 mm³, about 70,000 mm³ to about 80,000 mm³, about 70,000 mm³ to about 90,000 mm³, about 70,000 mm³ to about 100,000 mm³, about 80,000 mm³ to about 90,000 mm³, about 80,000 mm³ to about 100,000 mm³, or about 90,000 mm³ to about 100,000 mm³.

Turning to the proximal end 22 of the endoscope device 20 at which the secondary lumen 60 is formed contiguous with the proximal opening 32, in aspects of this embodiment, the length of the secondary lumen 60 is, e.g., about 8 mm, about 10 mm, about 12 mm, about 14 mm, about 16 mm, about 18 mm, about 20 mm, about 22 mm, about 24 mm, about 26 mm, about 28 mm, about 30 mm, about 35 mm, about 40 mm, about 45 mm, about 50 mm, about 55 mm, or about 60 mm. In other aspects of this embodiment, in an endoscope device 20 disclosed herein the length of the secondary lumen 60 is, e.g., at least 8 mm, at least 10 mm, at least 12 mm, at least 14 mm, at least 16 mm, at least 18 mm, at least 20 mm, at least 22 mm, at least 24 mm, at least 26 mm, at least 28 mm, at least 30 mm, at least 35 mm, at least 40 mm, at least 45 mm, at least 50 mm, at least 55 mm, or at least 60 mm. In still other aspects of this embodiment, in an endoscope device 20 disclosed herein the length of the secondary lumen 60 is, e.g., at most 8 mm, at most 10 mm, at most 12 mm, at most 14 mm, at most 16 mm, at most 18 mm, at most 20 mm, at most 22 mm, at most 24 mm, at most 26 mm, at most 28 mm, at most 30 mm, at most 35 mm, at most 40 mm, at most 45 mm, at most 50 mm, at most 55 mm, or at most 60 mm.

In other aspects of this embodiment, in an endoscope device 20 disclosed herein the length of the secondary lumen 60 is, e.g., about 8 mm to about 10 mm, about 8 mm to about 12 mm, about 8 mm to about 14 mm, about 8 mm to about 16 mm, about 8 mm to about 18 mm, about 8 mm to about 20 mm, about 8 mm to about 22 mm, about 8 mm to about 24 mm, about 8 mm to about 26 mm, about 8 mm to about 28 mm, about 8 mm to about 30 mm, about 8 mm to about 35 mm, about 8 mm to about 40 mm, about 8 mm to about 45 mm, about 8 mm to about 50 mm, about 8 mm to about 55 mm, about 8 mm to about 60 mm, about 10 mm to about 12 mm, about 10 mm to about 14 mm, about 10 mm to about 16 mm, about 10 mm to about 18 mm, about 10 mm to about 20 mm, about 10 mm to about 22 mm, about 10 mm to about 24 mm, about 10 mm to about 26 mm, about 10 mm to about 28 mm, about 10 mm to about 30 mm, about 10 mm to about 35 mm, about 10 mm to about 40 mm, about 10 mm to about 45 mm, about 10 mm to about 50 mm, about 10 mm to about 55 mm, about 10 mm to about 60 mm, about 12 mm to about 14 mm, about 12 mm to about 16 mm, about 12 mm to about 18 mm, about 12 mm to about 20 mm, about 12 mm to about 22 mm, about 12 mm to about 24 mm, about 12 mm to about 26 mm, about 12 mm to about 28 mm, about 12 mm to about 30 mm, about 12 mm to about 35 mm, about 12 mm to about 40 mm, about 12 mm to about 45 mm, about 12 mm to about 50 mm, about 12 mm to about 55 mm, about 12 mm to about 60 mm, about 14 mm to about 16 mm, about 14 mm to about 18 mm, about 14 mm to about 20 mm, about 14 mm to about 22 mm, about 14 mm to about 24 mm, about 14 mm to about 26 mm, about 14 mm to about 28 mm, about 14 mm to about 30 mm, about 14 mm to about 35 mm, about 14 mm to about 40 mm, about 14 mm to about 45 mm, about 14 mm to about 50 mm, about 14 mm to about 55 mm, about 14 mm to about 60 mm, about 16 mm to about 18 mm, about 16 mm to about 20 mm, about 16 mm to about 22 mm, about 16 mm to about 24 mm, about 16 mm to about 26 mm, about 16 mm to about 28 mm, about 16 mm to about 30 mm, about 16 mm to about 35 mm, about 16 mm to about 40 mm, about 16 mm to about 45 mm, about 16 mm to about 50 mm, about 16 mm to about 55 mm, about 16 mm to about 60 mm, about 18 mm to about 20 mm, about 18 mm to about 22 mm, about 18 mm to about 24 mm, about 18 mm to about 26 mm, about 18 mm to about 28 mm, about 18 mm to about 30 mm, about 18 mm to about 35 mm, about 18 mm to about 40 mm, about 18 mm to about 45 mm, about 18 mm to about 50 mm, about 18 mm to about 55 mm, about 18 mm to about 60 mm, about 20 mm to about 22 mm, about 20 mm to about 24 mm, about 20 mm to about 26 mm, about 20 mm to about 28 mm, about 20 mm to about 30 mm, about 20 mm to about 35 mm, about 20 mm to about 40 mm, about 20 mm to about 45 mm, about 20 mm to about 50 mm, about 20 mm to about 55 mm, about 20 mm to about 60 mm, about 22 mm to about 24 mm, about 22 mm to about 26 mm, about 22 mm to about 28 mm, about 22 mm to about 30 mm, about 22 mm to about 35 mm, about 22 mm to about 40 mm, about 22 mm to about 45 mm, about 22 mm to about 50 mm, about 22 mm to about 55 mm, about 22 mm to about 60 mm, about 24 mm to about 26 mm, about 24 mm to about 28 mm, about 24 mm to about 30 mm, about 24 mm to about 35 mm, about 24 mm to about 40 mm, about 24 mm to about 45 mm, about 24 mm to about 50 mm, about 24 mm to about 55 mm, about 24 mm to about 60 mm, about 26 mm to about 28 mm, about 26 mm to about 30 mm, about 26 mm to about 35 mm, about 26 mm to about 40 mm, about 26 mm to about 45 mm, about 26 mm to about 50 mm, about 26 mm to about 55 mm, about 26 mm to about 60 mm, about 28 mm to about 30 mm, about 28 mm to about 35 mm, about 28 mm to about 40 mm, about 28 mm to about 45 mm, about 28 mm to about 50 mm, about 28 mm to about 55 mm, about 28 mm to about 60 mm, about 30 mm to about 35 mm, about 30 mm to about 40 mm, about 30 mm to about 45 mm, about 30 mm to about 50 mm, about 30 mm to about 55 mm, about 30 mm to about 60 mm, about 35 mm to about 40 mm, about 35 mm to about 45 mm, about 35 mm to about 50 mm, about 35 mm to about 55 mm, about 35 mm to about 60 mm, about 40 mm to about 45 mm, about 40 mm to about 50 mm, about 40 mm to about 55 mm, about 40 mm to about 60 mm, about 45 mm to about 50 mm, about 45 mm to about 55 mm, about 45 mm to about 60 mm, about 50 mm to about 55 mm, about 50 mm to about 60 mm, or about 55 mm to about 60 mm.

In further aspects of this embodiment, the width of the secondary lumen 60 is, e.g., about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, or about 30 mm. In still further aspects of this embodiment, the width of the secondary lumen 60 is, e.g., at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, at least 21 mm, at least 22 mm, at least 23 mm, at least 24 mm, at least 25 mm, at least 26 mm, at least 27 mm, at least 28 mm, at least 29 mm, or at least 30 mm. In still further aspects of this embodiment, the width of the secondary lumen 60 is, e.g., at most 4 mm, at most 5 mm, at most 6 mm, at most 7 mm, at most 8 mm, at most 9 mm, at most 10 mm, at most 11 mm, at most 12 mm, at most 13 mm, at most 14 mm, at most 15 mm, at most 16 mm, at most 17 mm, at most 18 mm, at most 19 mm, at most 20 mm, at most 21 mm, at most 22 mm, at most 23 mm, at most 24 mm, at most 25 mm, at most 26 mm, at most 27 mm, at most 28 mm, at most 29 mm, or at most 30 mm.

In other aspects of this embodiment, in an endoscope device 20 disclosed herein the width of the secondary lumen 60 is, e.g., about 4 mm to about 5 mm, about 4 mm to about 6 mm, about 4 mm to about 7 mm, about 4 mm to about 8 mm, about 4 mm to about 9 mm, about 4 mm to about 10 mm, about 4 mm to about 11 mm, about 4 mm to about 12 mm, about 4 mm to about 13 mm, about 4 mm to about 14 mm, about 4 mm to about 15 mm, about 4 mm to about 16 mm, about 4 mm to about 17 mm, about 4 mm to about 18 mm, about 4 mm to about 19 mm, about 4 mm to about 20 mm, about 4 mm to about 21 mm, about 4 mm to about 22 mm, about 4 mm to about 23 mm, about 4 mm to about 24 mm, about 4 mm to about 25 mm, about 4 mm to about 26 mm, about 4 mm to about 27 mm, about 4 mm to about 28 mm, about 4 mm to about 29 mm, about 4 mm to about 30 mm, about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 5 mm to about 13 mm, about 5 mm to about 14 mm, about 5 mm to about 15 mm, about 5 mm to about 16 mm, about 5 mm to about 17 mm, about 5 mm to about 18 mm, about 5 mm to about 19 mm, about 5 mm to about 20 mm, about 5 mm to about 21 mm, about 5 mm to about 22 mm, about 5 mm to about 23 mm, about 5 mm to about 24 mm, about 5 mm to about 25 mm, about 5 mm to about 26 mm, about 5 mm to about 27 mm, about 5 mm to about 28 mm, about 5 mm to about 29 mm, about 5 mm to about 30 mm, about 6 mm to about 7 mm, about 6 mm to about 8 mm, about 6 mm to about 9 mm, about 6 mm to about 10 mm, about 6 mm to about 11 mm, about 6 mm to about 12 mm, about 6 mm to about 13 mm, about 6 mm to about 14 mm, about 6 mm to about 15 mm, about 6 mm to about 16 mm, about 6 mm to about 17 mm, about 6 mm to about 18 mm, about 6 mm to about 19 mm, about 6 mm to about 20 mm, about 6 mm to about 21 mm, about 6 mm to about 22 mm, about 6 mm to about 23 mm, about 6 mm to about 24 mm, about 6 mm to about 25 mm, about 6 mm to about 26 mm, about 6 mm to about 27 mm, about 6 mm to about 28 mm, about 6 mm to about 29 mm, about 6 mm to about 30 mm, about 7 mm to about 8 mm, about 7 mm to about 9 mm, about 7 mm to about 10 mm, about 7 mm to about 11 mm, about 7 mm to about 12 mm, about 7 mm to about 13 mm, about 7 mm to about 14 mm, about 7 mm to about 15 mm, about 7 mm to about 16 mm, about 7 mm to about 17 mm, about 7 mm to about 18 mm, about 7 mm to about 19 mm, about 7 mm to about 20 mm, about 7 mm to about 21 mm, about 7 mm to about 22 mm, about 7 mm to about 23 mm, about 7 mm to about 24 mm, about 7 mm to about 25 mm, about 7 mm to about 26 mm, about 7 mm to about 27 mm, about 7 mm to about 28 mm, about 7 mm to about 29 mm, about 7 mm to about 30 mm, about 8 mm to about 9 mm, about 8 mm to about 10 mm, about 8 mm to about 11 mm, about 8 mm to about 12 mm, about 8 mm to about 13 mm, about 8 mm to about 14 mm, about 8 mm to about 15 mm, about 8 mm to about 16 mm, about 8 mm to about 17 mm, about 8 mm to about 18 mm, about 8 mm to about 19 mm, about 8 mm to about 20 mm, about 8 mm to about 21 mm, about 8 mm to about 22 mm, about 8 mm to about 23 mm, about 8 mm to about 24 mm, about 8 mm to about 25 mm, about 8 mm to about 26 mm, about 8 mm to about 27 mm, about 8 mm to about 28 mm, about 8 mm to about 29 mm, about 8 mm to about 30 mm, about 9 mm to about 10 mm, about 9 mm to about 11 mm, about 9 mm to about 12 mm, about 9 mm to about 13 mm, about 9 mm to about 14 mm, about 9 mm to about 15 mm, about 9 mm to about 16 mm, about 9 mm to about 17 mm, about 9 mm to about 18 mm, about 9 mm to about 19 mm, about 9 mm to about 20 mm, about 9 mm to about 21 mm, about 9 mm to about 22 mm, about 9 mm to about 23 mm, about 9 mm to about 29 mm, about 9 mm to about 25 mm, about 9 mm to about 26 mm, about 9 mm to about 27 mm, about 9 mm to about 28 mm, about 9 mm to about 29 mm, about 9 mm to about 30 mm, about 10 mm to about 11 mm, about 10 mm to about 12 mm, about 10 mm to about 13 mm, about 10 mm to about 14 mm, about 10 mm to about 15 mm, about 10 mm to about 16 mm, about 10 mm to about 17 mm, about 10 mm to about 18 mm, about 10 mm to about 19 mm, about 10 mm to about 20 mm, about 10 mm to about 21 mm, about 10 mm to about 22 mm, about 10 mm to about 23 mm, about 10 mm to about 24 mm, about 10 mm to about 25 mm, about 10 mm to about 26 mm, about 10 mm to about 27 mm, about 10 mm to about 28 mm, about 10 mm to about 29 mm, about 10 mm to about 30 mm, about 11 mm to about 12 mm, about 11 mm to about 13 mm, about 11 mm to about 14 mm, about 11 mm to about 15 mm, about 11 mm to about 16 mm, about 11 mm to about 17 mm, about 11 mm to about 18 mm, about 11 mm to about 19 mm, about 11 mm to about 20 mm, about 11 mm to about 21 mm, about 11 mm to about 22 mm, about 11 mm to about 23 mm, about 11 mm to about 24 mm, about 11 mm to about 25 mm, about 11 mm to about 26 mm, about 11 mm to about 27 mm, about 11 mm to about 28 mm, about 11 mm to about 29 mm, about 11 mm to about 30 mm, about 12 mm to about 13 mm, about 12 mm to about 14 mm, about 12 mm to about 15 mm, about 12 mm to about 16 mm, about 12 mm to about 17 mm, about 12 mm to about 18 mm, about 12 mm to about 19 mm, about 12 mm to about 20 mm, about 12 mm to about 21 mm, about 12 mm to about 22 mm, about 12 mm to about 23 mm, about 12 mm to about 24 mm, about 12 mm to about 25 mm, about 12 mm to about 26 mm, about 12 mm to about 27 mm, about 12 mm to about 28 mm, about 12 mm to about 29 mm, about 12 mm to about 30 mm, about 13 mm to about 14 mm, about 13 mm to about 15 mm, about 13 mm to about 16 mm, about 13 mm to about 17 mm, about 13 mm to about 18 mm, about 13 mm to about 19 mm, about 13 mm to about 20 mm, about 13 mm to about 21 mm, about 13 mm to about 22 mm, about 13 mm to about 23 mm, about 13 mm to about 24 mm, about 13 mm to about 25 mm, about 13 mm to about 26 mm, about 13 mm to about 27 mm, about 13 mm to about 28 mm, about 13 mm to about 29 mm, about 13 mm to about 30 mm, about 14 mm to about 15 mm, about 14 mm to about 16 mm, about 14 mm to about 17 mm, about 14 mm to about 18 mm, about 14 mm to about 19 mm, about 14 mm to about 20 mm, about 14 mm to about 21 mm, about 14 mm to about 22 mm, about 14 mm to about 23 mm, about 14 mm to about 24 mm, about 14 mm to about 25 mm, about 14 mm to about 26 mm, about 14 mm to about 27 mm, about 14 mm to about 28 mm, about 14 mm to about 29 mm, about 14 mm to about 30 mm, about 15 mm to about 16 mm, about 15 mm to about 17 mm, about 15 mm to about 18 mm, about 15 mm to about 19 mm, about 15 mm to about 20 mm, about 15 mm to about 21 mm, about 15 mm to about 22 mm, about 15 mm to about 23 mm, about 15 mm to about 24 mm, about 15 mm to about 25 mm, about 15 mm to about 26 mm, about 15 mm to about 27 mm, about 15 mm to about 28 mm, about 15 mm to about 29 mm, about 15 mm to about 30 mm, about 16 mm to about 17 mm, about 16 mm to about 18 mm, about 16 mm to about 19 mm, about 16 mm to about 20 mm, about 16 mm to about 21 mm, about 16 mm to about 22 mm, about 16 mm to about 23 mm, about 16 mm to about 24 mm, about 16 mm to about 25 mm, about 16 mm to about 26 mm, about 16 mm to about 27 mm, about 16 mm to about 28 mm, about 16 mm to about 29 mm, about 16 mm to about 30 mm, about 17 mm to about 18 mm, about 17 mm to about 19 mm, about 17 mm to about 20 mm, about 17 mm to about 21 mm, about 17 mm to about 22 mm, about 17 mm to about 23 mm, about 17 mm to about 24 mm, about 17 mm to about 25 mm, about 17 mm to about 26 mm, about 17 mm to about 27 mm, about 17 mm to about 28 mm, about 17 mm to about 29 mm, about 17 mm to about 30 mm, about 18 mm to about 19 mm, about 18 mm to about 20 mm, about 18 mm to about 21 mm, about 18 mm to about 22 mm, about 18 mm to about 23 mm, about 18 mm to about 24 mm, about 18 mm to about 25 mm, about 18 mm to about 26 mm, about 18 mm to about 27 mm, about 18 mm to about 28 mm, about 18 mm to about 29 mm, about 18 mm to about 30 mm, about 19 mm to about 20 mm, about 19 mm to about 21 mm, about 19 mm to about 22 mm, about 19 mm to about 23 mm, about 19 mm to about 24 mm, about 19 mm to about 25 mm, about 19 mm to about 26 mm, about 19 mm to about 27 mm, about 19 mm to about 28 mm, about 19 mm to about 29 mm, about 19 mm to about 30 mm, about 20 mm to about 21 mm, about 20 mm to about 22 mm, about 20 mm to about 23 mm, about 20 mm to about 24 mm, about 20 mm to about 25 mm, about 20 mm to about 26 mm, about 20 mm to about 27 mm, about 20 mm to about 28 mm, about 20 mm to about 29 mm, about 20 mm to about 30 mm, about 21 mm to about 22 mm, about 21 mm to about 23 mm, about 21 mm to about 24 mm, about 21 mm to about 25 mm, about 21 mm to about 26 mm, about 21 mm to about 27 mm, about 21 mm to about 28 mm, about 21 mm to about 29 mm, about 21 mm to about 30 mm, about 22 mm to about 23 mm, about 22 mm to about 24 mm, about 22 mm to about 25 mm, about 22 mm to about 26 mm, about 22 mm to about 27 mm, about 22 mm to about 28 mm, about 22 mm to about 29 mm, about 22 mm to about 30 mm, about 23 mm to about 24 mm, about 23 mm to about 25 mm, about 23 mm to about 26 mm, about 23 mm to about 27 mm, about 23 mm to about 28 mm, about 23 mm to about 29 mm, about 23 mm to about 30 mm, about 24 mm to about 25 mm, about 24 mm to about 26 mm, about 24 mm to about 27 mm, about 24 mm to about 28 mm, about 24 mm to about 29 mm, about 24 mm to about 30 mm, about 25 mm to about 26 mm, about 25 mm to about 27 mm, about 25 mm to about 28 mm, about 25 mm to about 29 mm, about 25 mm to about 30 mm, about 26 mm to about 27 mm, about 26 mm to about 28 mm, about 26 mm to about 29 mm, about 26 mm to about 30 mm, about 27 mm to about 28 mm, about 27 mm to about 29 mm, about 27 mm to about 30 mm, about 28 mm to about 29 mm, about 28 mm to about 30 mm, or about 29 mm to about 30 mm.

Finally, in other aspects of this embodiment, in an endoscope device 20 wherein the effective wall thickness of the body wall 40 of the body 30 is defined by the distance at any location between the outer surface 42 and the opposite inner surface 44, the wall thickness is, e.g., about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, or about 5.0 mm. In still further aspects of this embodiment, the wall thickness of the body wall 40 is, e.g., at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3.0 mm, at least 3.2 mm, at least 3.4 mm, at least 3.6 mm, at least 3.8 mm, at least 4.0 mm, at least 4.2 mm, at least 4.4 mm, at least 4.6 mm, at least 4.8 mm, or at least 5.0 mm. In still further aspects of this embodiment, the wall thickness of the body wall 40 is, e.g., at most 0.1 mm, at most 0.2 mm, at most 0.3 mm, at most 0.4 mm, at most 0.5 mm, at most 0.6 mm, at most 0.7 mm, at most 0.8 mm, at most 0.9 mm, at most 1.0 mm, at most 1.1 mm, at most 1.2 mm, at most 1.3 mm, at most 1.4 mm, at most 1.5 mm, at most 1.6 mm, at most 1.7 mm, at most 1.8 mm, at most 1.9 mm, at most 2.0 mm, at most 2.1 mm, at most 2.2 mm, at most 2.3 mm, at most 2.4 mm, at most 2.5 mm, at most 2.6 mm, at most 2.7 mm, at most 2.8 mm, at most 2.9 mm, at most 3.0 mm, at most 3.2 mm, at most 3.4 mm, at most 3.6 mm, at most 3.8 mm, at most 4.0 mm, at most 4.2 mm, at most 4.4 mm, at most 4.6 mm, at most 4.8 mm, or at most 5.0 mm.

In other aspects of this embodiment, in an endoscope device 20 disclosed herein the wall thickness of the body wall 40 is, e.g., about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.7 mm, about 0.1 mm to about 0.8 mm, about 0.1 mm to about 0.9 mm, about 0.1 mm to about 1.0 mm, about 0.1 mm to about 1.1 mm, about 0.1 mm to about 1.2 mm, about 0.1 mm to about 1.3 mm, about 0.1 mm to about 1.4 mm, about 0.1 mm to about 1.5 mm, about 0.1 mm to about 1.6 mm, about 0.1 mm to about 1.7 mm, about 0.1 mm to about 1.8 mm, about 0.1 mm to about 1.9 mm, about 0.1 mm to about 2.0 mm, about 0.1 mm to about 2.1 mm, about 0.1 mm to about 2.2 mm, about 0.1 mm to about 2.3 mm, about 0.1 mm to about 2.4 mm, about 0.1 mm to about 2.5 mm, about 0.1 mm to about 2.6 mm, about 0.1 mm to about 2.7 mm, about 0.1 mm to about 2.8 mm, about 0.1 mm to about 2.9 mm, about 0.1 mm to about 3.0 mm, about 0.1 mm to about 3.2 mm, about 0.1 mm to about 3.4 mm, about 0.1 mm to about 3.6 mm, about 0.1 mm to about 3.8 mm, about 0.1 mm to about 4.0 mm, about 0.1 mm to about 4.2 mm, about 0.1 mm to about 4.4 mm, about 0.1 mm to about 4.6 mm, about 0.1 mm to about 4.8 mm, about 0.1 mm to about 5.0 mm, about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1.0 mm, about 0.2 mm to about 1.1 mm, about 0.2 mm to about 1.2 mm, about 0.2 mm to about 1.3 mm, about 0.2 mm to about 1.4 mm, about 0.2 mm to about 1.5 mm, about 0.2 mm to about 1.6 mm, about 0.2 mm to about 1.7 mm, about 0.2 mm to about 1.8 mm, about 0.2 mm to about 1.9 mm, about 0.2 mm to about 2.0 mm, about 0.2 mm to about 2.1 mm, about 0.2 mm to about 2.2 mm, about 0.2 mm to about 2.3 mm, about 0.2 mm to about 2.4 mm, about 0.2 mm to about 2.5 mm, about 0.2 mm to about 2.6 mm, about 0.2 mm to about 2.7 mm, about 0.2 mm to about 2.8 mm, about 0.2 mm to about 2.9 mm, about 0.2 mm to about 3.0 mm, about 0.2 mm to about 3.2 mm, about 0.2 mm to about 3.4 mm, about 0.2 mm to about 3.6 mm, about 0.2 mm to about 3.8 mm, about 0.2 mm to about 4.0 mm, about 0.2 mm to about 4.2 mm, about 0.2 mm to about 4.4 mm, about 0.2 mm to about 4.6 mm, about 0.2 mm to about 4.8 mm, about 0.2 mm to about 5.0 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1.0 mm, about 0.3 mm to about 1.1 mm, about 0.3 mm to about 1.2 mm, about 0.3 mm to about 1.3 mm, about 0.3 mm to about 1.4 mm, about 0.3 mm to about 1.5 mm, about 0.3 mm to about 1.6 mm, about 0.3 mm to about 1.7 mm, about 0.3 mm to about 1.8 mm, about 0.3 mm to about 1.9 mm, about 0.3 mm to about 2.0 mm, about 0.3 mm to about 2.1 mm, about 0.3 mm to about 2.2 mm, about 0.3 mm to about 2.3 mm, about 0.3 mm to about 2.4 mm, about 0.3 mm to about 2.5 mm, about 0.3 mm to about 2.6 mm, about 0.3 mm to about 2.7 mm, about 0.3 mm to about 2.8 mm, about 0.3 mm to about 2.9 mm, about 0.3 mm to about 3.0 mm, about 0.3 mm to about 3.2 mm, about 0.3 mm to about 3.4 mm, about 0.3 mm to about 3.6 mm, about 0.3 mm to about 3.8 mm, about 0.3 mm to about 4.0 mm, about 0.3 mm to about 4.2 mm, about 0.3 mm to about 4.4 mm, about 0.3 mm to about 4.6 mm, about 0.3 mm to about 4.8 mm, about 0.3 mm to about 5.0 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1.0 mm, about 0.4 mm to about 1.1 mm, about 0.4 mm to about 1.2 mm, about 0.4 mm to about 1.3 mm, about 0.4 mm to about 1.4 mm, about 0.4 mm to about 1.5 mm, about 0.4 mm to about 1.6 mm, about 0.4 mm to about 1.7 mm, about 0.4 mm to about 1.8 mm, about 0.4 mm to about 1.9 mm, about 0.4 mm to about 2.0 mm, about 0.4 mm to about 2.1 mm, about 0.4 mm to about 2.2 mm, about 0.4 mm to about 2.3 mm, about 0.4 mm to about 2.4 mm, about 0.4 mm to about 2.5 mm, about 0.4 mm to about 2.6 mm, about 0.4 mm to about 2.7 mm, about 0.4 mm to about 2.8 mm, about 0.4 mm to about 2.9 mm, about 0.4 mm to about 3.0 mm, about 0.4 mm to about 3.2 mm, about 0.4 mm to about 3.4 mm, about 0.4 mm to about 3.6 mm, about 0.4 mm to about 3.8 mm, about 0.4 mm to about 4.0 mm, about 0.4 mm to about 4.2 mm, about 0.4 mm to about 4.4 mm, about 0.4 mm to about 4.6 mm, about 0.4 mm to about 4.8 mm, about 0.4 mm to about 5.0 mm, about 0.5 mm to about 0.6 mm, about 0.5 mm to about 0.7 mm, about 0.5 mm to about 0.8 mm, about 0.5 mm to about 0.9 mm, about 0.5 mm to about 1.0 mm, about 0.5 mm to about 1.1 mm, about 0.5 mm to about 1.2 mm, about 0.5 mm to about 1.3 mm, about 0.5 mm to about 1.4 mm, about 0.5 mm to about 1.5 mm, about 0.5 mm to about 1.6 mm, about 0.5 mm to about 1.7 mm, about 0.5 mm to about 1.8 mm, about 0.5 mm to about 1.9 mm, about 0.5 mm to about 2.0 mm, about 0.5 mm to about 2.1 mm, about 0.5 mm to about 2.2 mm, about 0.5 mm to about 2.3 mm, about 0.5 mm to about 2.4 mm, about 0.5 mm to about 2.5 mm, about 0.5 mm to about 2.6 mm, about 0.5 mm to about 2.7 mm, about 0.5 mm to about 2.8 mm, about 0.5 mm to about 2.9 mm, about 0.5 mm to about 3.0 mm, about 0.5 mm to about 3.2 mm, about 0.5 mm to about 3.4 mm, about 0.5 mm to about 3.6 mm, about 0.5 mm to about 3.8 mm, about 0.5 mm to about 4.0 mm, about 0.5 mm to about 4.2 mm, about 0.5 mm to about 4.4 mm, about 0.5 mm to about 4.6 mm, about 0.5 mm to about 4.8 mm, about 0.5 mm to about 5.0 mm, about 0.6 mm to about 0.7 mm, about 0.6 mm to about 0.8 mm, about 0.6 mm to about 0.9 mm, about 0.6 mm to about 1.0 mm, about 0.6 mm to about 1.1 mm, about 0.6 mm to about 1.2 mm, about 0.6 mm to about 1.3 mm, about 0.6 mm to about 1.4 mm, about 0.6 mm to about 1.5 mm, about 0.6 mm to about 1.6 mm, about 0.6 mm to about 1.7 mm, about 0.6 mm to about 1.8 mm, about 0.6 mm to about 1.9 mm, about 0.6 mm to about 2.0 mm, about 0.6 mm to about 2.1 mm, about 0.6 mm to about 2.2 mm, about 0.6 mm to about 2.3 mm, about 0.6 mm to about 2.4 mm, about 0.6 mm to about 2.5 mm, about 0.6 mm to about 2.6 mm, about 0.6 mm to about 2.7 mm, about 0.6 mm to about 2.8 mm, about 0.6 mm to about 2.9 mm, about 0.6 mm to about 3.0 mm, about 0.6 mm to about 3.2 mm, about 0.6 mm to about 3.4 mm, about 0.6 mm to about 3.6 mm, about 0.6 mm to about 3.8 mm, about 0.6 mm to about 4.0 mm, about 0.6 mm to about 4.2 mm, about 0.6 mm to about 4.4 mm, about 0.6 mm to about 4.6 mm, about 0.6 mm to about 4.8 mm, about 0.6 mm to about 5.0 mm, about 0.7 mm to about 0.8 mm, about 0.7 mm to about 0.9 mm, about 0.7 mm to about 1.0 mm, about 0.7 mm to about 1.1 mm, about 0.7 mm to about 1.2 mm, about 0.7 mm to about 1.3 mm, about 0.7 mm to about 1.4 mm, about 0.7 mm to about 1.5 mm, about 0.7 mm to about 1.6 mm, about 0.7 mm to about 1.7 mm, about 0.7 mm to about 1.8 mm, about 0.7 mm to about 1.9 mm, about 0.7 mm to about 2.0 mm, about 0.7 mm to about 2.1 mm, about 0.7 mm to about 2.2 mm, about 0.7 mm to about 2.3 mm, about 0.7 mm to about 2.4 mm, about 0.7 mm to about 2.5 mm, about 0.7 mm to about 2.6 mm, about 0.7 mm to about 2.7 mm, about 0.7 mm to about 2.8 mm, about 0.7 mm to about 2.9 mm, about 0.7 mm to about 3.0 mm, about 0.7 mm to about 3.2 mm, about 0.7 mm to about 3.4 mm, about 0.7 mm to about 3.6 mm, about 0.7 mm to about 3.8 mm, about 0.7 mm to about 4.0 mm, about 0.7 mm to about 4.2 mm, about 0.7 mm to about 4.4 mm, about 0.7 mm to about 4.6 mm, about 0.7 mm to about 4.8 mm, about 0.7 mm to about 5.0 mm, about 0.8 mm to about 0.9 mm, about 0.8 mm to about 1.0 mm, about 0.8 mm to about 1.1 mm, about 0.8 mm to about 1.2 mm, about 0.8 mm to about 1.3 mm, about 0.8 mm to about 1.4 mm, about 0.8 mm to about 1.5 mm, about 0.8 mm to about 1.6 mm, about 0.8 mm to about 1.7 mm, about 0.8 mm to about 1.8 mm, about 0.8 mm to about 1.9 mm, about 0.8 mm to about 2.0 mm, about 0.8 mm to about 2.1 mm, about 0.8 mm to about 2.2 mm, about 0.8 mm to about 2.3 mm, about 0.8 mm to about 2.4 mm, about 0.8 mm to about 2.5 mm, about 0.8 mm to about 2.6 mm, about 0.8 mm to about 2.7 mm, about 0.8 mm to about 2.8 mm, about 0.8 mm to about 2.9 mm, about 0.8 mm to about 3.0 mm, about 0.8 mm to about 3.2 mm, about 0.8 mm to about 3.4 mm, about 0.8 mm to about 3.6 mm, about 0.8 mm to about 3.8 mm, about 0.8 mm to about 4.0 mm, about 0.8 mm to about 4.2 mm, about 0.8 mm to about 4.4 mm, about 0.8 mm to about 4.6 mm, about 0.8 mm to about 4.8 mm, about 0.8 mm to about 5.0 mm, about 0.9 mm to about 1.0 mm, about 0.9 mm to about 1.1 mm, about 0.9 mm to about 1.2 mm, about 0.9 mm to about 1.3 mm, about 0.9 mm to about 1.4 mm, about 0.9 mm to about 1.5 mm, about 0.9 mm to about 1.6 mm, about 0.9 mm to about 1.7 mm, about 0.9 mm to about 1.8 mm, about 0.9 mm to about 1.9 mm, about 0.9 mm to about 2.0 mm, about 0.9 mm to about 2.1 mm, about 0.9 mm to about 2.2 mm, about 0.9 mm to about 2.3 mm, about 0.9 mm to about 2.4 mm, about 0.9 mm to about 2.5 mm, about 0.9 mm to about 2.6 mm, about 0.9 mm to about 2.7 mm, about 0.9 mm to about 2.8 mm, about 0.9 mm to about 2.9 mm, about 0.9 mm to about 3.0 mm, about 0.9 mm to about 3.2 mm, about 0.9 mm to about 3.4 mm, about 0.9 mm to about 3.6 mm, about 0.9 mm to about 3.8 mm, about 0.9 mm to about 4.0 mm, about 0.9 mm to about 4.2 mm, about 0.9 mm to about 4.4 mm, about 0.9 mm to about 4.6 mm, about 0.9 mm to about 4.8 mm, about 0.9 mm to about 5.0 mm, about 1.0 mm to about 1.1 mm, about 1.0 mm to about 1.2 mm, about 1.0 mm to about 1.3 mm, about 1.0 mm to about 1.4 mm, about 1.0 mm to about 1.5 mm, about 1.0 mm to about 1.6 mm, about 1.0 mm to about 1.7 mm, about 1.0 mm to about 1.8 mm, about 1.0 mm to about 1.9 mm, about 1.0 mm to about 2.0 mm, about 1.0 mm to about 2.1 mm, about 1.0 mm to about 2.2 mm, about 1.0 mm to about 2.3 mm, about 1.0 mm to about 2.4 mm, about 1.0 mm to about 2.5 mm, about 1.0 mm to about 2.6 mm, about 1.0 mm to about 2.7 mm, about 1.0 mm to about 2.8 mm, about 1.0 mm to about 2.9 mm, about 1.0 mm to about 3.0 mm, about 1.0 mm to about 3.2 mm, about 1.0 mm to about 3.4 mm, about 1.0 mm to about 3.6 mm, about 1.0 mm to about 3.8 mm, about 1.0 mm to about 4.0 mm, about 1.0 mm to about 4.2 mm, about 1.0 mm to about 4.4 mm, about 1.0 mm to about 4.6 mm, about 1.0 mm to about 4.8 mm, about 1.0 mm to about 5.0 mm, about 1.1 mm to about 1.2 mm, about 1.1 mm to about 1.3 mm, about 1.1 mm to about 1.4 mm, about 1.1 mm to about 1.5 mm, about 1.1 mm to about 1.6 mm, about 1.1 mm to about 1.7 mm, about 1.1 mm to about 1.8 mm, about 1.1 mm to about 1.9 mm, about 1.1 mm to about 2.0 mm, about 1.1 mm to about 2.1 mm, about 1.1 mm to about 2.2 mm, about 1.1 mm to about 2.3 mm, about 1.1 mm to about 2.4 mm, about 1.1 mm to about 2.5 mm, about 1.1 mm to about 2.6 mm, about 1.1 mm to about 2.7 mm, about 1.1 mm to about 2.8 mm, about 1.1 mm to about 2.9 mm, about 1.1 mm to about 3.0 mm, about 1.1 mm to about 3.2 mm, about 1.1 mm to about 3.4 mm, about 1.1 mm to about 3.6 mm, about 1.1 mm to about 3.8 mm, about 1.1 mm to about 4.0 mm, about 1.1 mm to about 4.2 mm, about 1.1 mm to about 4.4 mm, about 1.1 mm to about 4.6 mm, about 1.1 mm to about 4.8 mm, about 1.1 mm to about 5.0 mm, about 1.2 mm to about 1.3 mm, about 1.2 mm to about 1.4 mm, about 1.2 mm to about 1.5 mm, about 1.2 mm to about 1.6 mm, about 1.2 mm to about 1.7 mm, about 1.2 mm to about 1.8 mm, about 1.2 mm to about 1.9 mm, about 1.2 mm to about 2.0 mm, about 1.2 mm to about 2.1 mm, about 1.2 mm to about 2.2 mm, about 1.2 mm to about 2.3 mm, about 1.2 mm to about 2.4 mm, about 1.2 mm to about 2.5 mm, about 1.2 mm to about 2.6 mm, about 1.2 mm to about 2.7 mm, about 1.2 mm to about 2.8 mm, about 1.2 mm to about 2.9 mm, about 1.2 mm to about 3.0 mm, about 1.2 mm to about 3.2 mm, about 1.2 mm to about 3.4 mm, about 1.2 mm to about 3.6 mm, about 1.2 mm to about 3.8 mm, about 1.2 mm to about 4.0 mm, about 1.2 mm to about 4.2 mm, about 1.2 mm to about 4.4 mm, about 1.2 mm to about 4.6 mm, about 1.2 mm to about 4.8 mm, about 1.2 mm to about 5.0 mm, about 1.3 mm to about 1.4 mm, about 1.3 mm to about 1.5 mm, about 1.3 mm to about 1.6 mm, about 1.3 mm to about 1.7 mm, about 1.3 mm to about 1.8 mm, about 1.3 mm to about 1.9 mm, about 1.3 mm to about 2.0 mm, about 1.3 mm to about 2.1 mm, about 1.3 mm to about 2.2 mm, about 1.3 mm to about 2.3 mm, about 1.3 mm to about 2.4 mm, about 1.3 mm to about 2.5 mm, about 1.3 mm to about 2.6 mm, about 1.3 mm to about 2.7 mm, about 1.3 mm to about 2.8 mm, about 1.3 mm to about 2.9 mm, about 1.3 mm to about 3.0 mm, about 1.3 mm to about 3.2 mm, about 1.3 mm to about 3.4 mm, about 1.3 mm to about 3.6 mm, about 1.3 mm to about 3.8 mm, about 1.3 mm to about 4.0 mm, about 1.3 mm to about 4.2 mm, about 1.3 mm to about 4.4 mm, about 1.3 mm to about 4.6 mm, about 1.3 mm to about 4.8 mm, about 1.3 mm to about 5.0 mm, about 1.4 mm to about 1.5 mm, about 1.4 mm to about 1.6 mm, about 1.4 mm to about 1.7 mm, about 1.4 mm to about 1.8 mm, about 1.4 mm to about 1.9 mm, about 1.4 mm to about 2.0 mm, about 1.4 mm to about 2.1 mm, about 1.4 mm to about 2.2 mm, about 1.4 mm to about 2.3 mm, about 1.4 mm to about 2.4 mm, about 1.4 mm to about 2.5 mm, about 1.4 mm to about 2.6 mm, about 1.4 mm to about 2.7 mm, about 1.4 mm to about 2.8 mm, about 1.4 mm to about 2.9 mm, about 1.4 mm to about 3.0 mm, about 1.4 mm to about 3.2 mm, about 1.4 mm to about 3.4 mm, about 1.4 mm to about 3.6 mm, about 1.4 mm to about 3.8 mm, about 1.4 mm to about 4.0 mm, about 1.4 mm to about 4.2 mm, about 1.4 mm to about 4.4 mm, about 1.4 mm to about 4.6 mm, about 1.4 mm to about 4.8 mm, about 1.4 mm to about 5.0 mm, about 1.5 mm to about 1.6 mm, about 1.5 mm to about 1.7 mm, about 1.5 mm to about 1.8 mm, about 1.5 mm to about 1.9 mm, about 1.5 mm to about 2.0 mm, about 1.5 mm to about 2.1 mm, about 1.5 mm to about 2.2 mm, about 1.5 mm to about 2.3 mm, about 1.5 mm to about 2.4 mm, about 1.5 mm to about 2.5 mm, about 1.5 mm to about 2.6 mm, about 1.5 mm to about 2.7 mm, about 1.5 mm to about 2.8 mm, about 1.5 mm to about 2.9 mm, about 1.5 mm to about 3.0 mm, about 1.5 mm to about 3.2 mm, about 1.5 mm to about 3.4 mm, about 1.5 mm to about 3.6 mm, about 1.5 mm to about 3.8 mm, about 1.5 mm to about 4.0 mm, about 1.5 mm to about 4.2 mm, about 1.5 mm to about 4.4 mm, about 1.5 mm to about 4.6 mm, about 1.5 mm to about 4.8 mm, about 1.5 mm to about 5.0 mm, about 1.6 mm to about 1.7 mm, about 1.6 mm to about 1.8 mm, about 1.6 mm to about 1.9 mm, about 1.6 mm to about 2.0 mm, about 1.6 mm to about 2.1 mm, about 1.6 mm to about 2.2 mm, about 1.6 mm to about 2.3 mm, about 1.6 mm to about 2.4 mm, about 1.6 mm to about 2.5 mm, about 1.6 mm to about 2.6 mm, about 1.6 mm to about 2.7 mm, about 1.6 mm to about 2.8 mm, about 1.6 mm to about 2.9 mm, about 1.6 mm to about 3.0 mm, about 1.6 mm to about 3.2 mm, about 1.6 mm to about 3.4 mm, about 1.6 mm to about 3.6 mm, about 1.6 mm to about 3.8 mm, about 1.6 mm to about 4.0 mm, about 1.6 mm to about 4.2 mm, about 1.6 mm to about 4.4 mm, about 1.6 mm to about 4.6 mm, about 1.6 mm to about 4.8 mm, about 1.6 mm to about 5.0 mm, about 1.7 mm to about 1.8 mm, about 1.7 mm to about 1.9 mm, about 1.7 mm to about 2.0 mm, about 1.7 mm to about 2.1 mm, about 1.7 mm to about 2.2 mm, about 1.7 mm to about 2.3 mm, about 1.7 mm to about 2.4 mm, about 1.7 mm to about 2.5 mm, about 1.7 mm to about 2.6 mm, about 1.7 mm to about 2.7 mm, about 1.7 mm to about 2.8 mm, about 1.7 mm to about 2.9 mm, about 1.7 mm to about 3.0 mm, about 1.7 mm to about 3.2 mm, about 1.7 mm to about 3.4 mm, about 1.7 mm to about 3.6 mm, about 1.7 mm to about 3.8 mm, about 1.7 mm to about 4.0 mm, about 1.7 mm to about 4.2 mm, about 1.7 mm to about 4.4 mm, about 1.7 mm to about 4.6 mm, about 1.7 mm to about 4.8 mm, about 1.7 mm to about 5.0 mm, about 1.8 mm to about 1.9 mm, about 1.8 mm to about 2.0 mm, about 1.8 mm to about 2.1 mm, about 1.8 mm to about 2.2 mm, about 1.8 mm to about 2.3 mm, about 1.8 mm to about 2.4 mm, about 1.8 mm to about 2.5 mm, about 1.8 mm to about 2.6 mm, about 1.8 mm to about 2.7 mm, about 1.8 mm to about 2.8 mm, about 1.8 mm to about 2.9 mm, about 1.8 mm to about 3.0 mm, about 1.8 mm to about 3.2 mm, about 1.8 mm to about 3.4 mm, about 1.8 mm to about 3.6 mm, about 1.8 mm to about 3.8 mm, about 1.8 mm to about 4.0 mm, about 1.8 mm to about 4.2 mm, about 1.8 mm to about 4.4 mm, about 1.8 mm to about 4.6 mm, about 1.8 mm to about 4.8 mm, about 1.8 mm to about 5.0 mm, about 1.9 mm to about 2.0 mm, about 1.9 mm to about 2.1 mm, about 1.9 mm to about 2.2 mm, about 1.9 mm to about 2.3 mm, about 1.9 mm to about 2.4 mm, about 1.9 mm to about 2.5 mm, about 1.9 mm to about 2.6 mm, about 1.9 mm to about 2.7 mm, about 1.9 mm to about 2.8 mm, about 1.9 mm to about 2.9 mm, about 1.9 mm to about 3.0 mm, about 1.9 mm to about 3.2 mm, about 1.9 mm to about 3.4 mm, about 1.9 mm to about 3.6 mm, about 1.9 mm to about 3.8 mm, about 1.9 mm to about 4.0 mm, about 1.9 mm to about 4.2 mm, about 1.9 mm to about 4.4 mm, about 1.9 mm to about 4.6 mm, about 1.9 mm to about 4.8 mm, about 1.9 mm to about 5.0 mm, about 2.0 mm to about 2.1 mm, about 2.0 mm to about 2.2 mm, about 2.0 mm to about 2.3 mm, about 2.0 mm to about 2.4 mm, about 2.0 mm to about 2.5 mm, about 2.0 mm to about 2.6 mm, about 2.0 mm to about 2.7 mm, about 2.0 mm to about 2.8 mm, about 2.0 mm to about 2.9 mm, about 2.0 mm to about 3.0 mm, about 2.0 mm to about 3.2 mm, about 2.0 mm to about 3.4 mm, about 2.0 mm to about 3.6 mm, about 2.0 mm to about 3.8 mm, about 2.0 mm to about 4.0 mm, about 2.0 mm to about 4.2 mm, about 2.0 mm to about 4.4 mm, about 2.0 mm to about 4.6 mm, about 2.0 mm to about 4.8 mm, about 2.0 mm to about 5.0 mm, about 2.1 mm to about 2.2 mm, about 2.1 mm to about 2.3 mm, about 2.1 mm to about 2.4 mm, about 2.1 mm to about 2.5 mm, about 2.1 mm to about 2.6 mm, about 2.1 mm to about 2.7 mm, about 2.1 mm to about 2.8 mm, about 2.1 mm to about 2.9 mm, about 2.1 mm to about 3.0 mm, about 2.1 mm to about 3.2 mm, about 2.1 mm to about 3.4 mm, about 2.1 mm to about 3.6 mm, about 2.1 mm to about 3.8 mm, about 2.1 mm to about 4.0 mm, about 2.1 mm to about 4.2 mm, about 2.1 mm to about 4.4 mm, about 2.1 mm to about 4.6 mm, about 2.1 mm to about 4.8 mm, about 2.1 mm to about 5.0 mm, about 2.2 mm to about 2.3 mm, about 2.2 mm to about 2.4 mm, about 2.2 mm to about 2.5 mm, about 2.2 mm to about 2.6 mm, about 2.2 mm to about 2.7 mm, about 2.2 mm to about 2.8 mm, about 2.2 mm to about 2.9 mm, about 2.2 mm to about 3.0 mm, about 2.2 mm to about 3.2 mm, about 2.2 mm to about 3.4 mm, about 2.2 mm to about 3.6 mm, about 2.2 mm to about 3.8 mm, about 2.2 mm to about 4.0 mm, about 2.2 mm to about 4.2 mm, about 2.2 mm to about 4.4 mm, about 2.2 mm to about 4.6 mm, about 2.2 mm to about 4.8 mm, about 2.2 mm to about 5.0 mm, about 2.3 mm to about 2.4 mm, about 2.3 mm to about 2.5 mm, about 2.3 mm to about 2.6 mm, about 2.3 mm to about 2.7 mm, about 2.3 mm to about 2.8 mm, about 2.3 mm to about 2.9 mm, about 2.3 mm to about 3.0 mm, about 2.3 mm to about 3.2 mm, about 2.3 mm to about 3.4 mm, about 2.3 mm to about 3.6 mm, about 2.3 mm to about 3.8 mm, about 2.3 mm to about 4.0 mm, about 2.3 mm to about 4.2 mm, about 2.3 mm to about 4.4 mm, about 2.3 mm to about 4.6 mm, about 2.3 mm to about 4.8 mm, about 2.3 mm to about 5.0 mm, about 2.4 mm to about 2.5 mm, about 2.4 mm to about 2.6 mm, about 2.4 mm to about 2.7 mm, about 2.4 mm to about 2.8 mm, about 2.4 mm to about 2.9 mm, about 2.4 mm to about 3.0 mm, about 2.4 mm to about 3.2 mm, about 2.4 mm to about 3.4 mm, about 2.4 mm to about 3.6 mm, about 2.4 mm to about 3.8 mm, about 2.4 mm to about 4.0 mm, about 2.4 mm to about 4.2 mm, about 2.4 mm to about 4.4 mm, about 2.4 mm to about 4.6 mm, about 2.4 mm to about 4.8 mm, about 2.4 mm to about 5.0 mm, about 2.5 mm to about 2.6 mm, about 2.5 mm to about 2.7 mm, about 2.5 mm to about 2.8 mm, about 2.5 mm to about 2.9 mm, about 2.5 mm to about 3.0 mm, about 2.5 mm to about 3.2 mm, about 2.5 mm to about 3.4 mm, about 2.5 mm to about 3.6 mm, about 2.5 mm to about 3.8 mm, about 2.5 mm to about 4.0 mm, about 2.5 mm to about 4.2 mm, about 2.5 mm to about 4.4 mm, about 2.5 mm to about 4.6 mm, about 2.5 mm to about 4.8 mm, about 2.5 mm to about 5.0 mm, about 2.6 mm to about 2.7 mm, about 2.6 mm to about 2.8 mm, about 2.6 mm to about 2.9 mm, about 2.6 mm to about 3.0 mm, about 2.6 mm to about 3.2 mm, about 2.6 mm to about 3.4 mm, about 2.6 mm to about 3.6 mm, about 2.6 mm to about 3.8 mm, about 2.6 mm to about 4.0 mm, about 2.6 mm to about 4.2 mm, about 2.6 mm to about 4.4 mm, about 2.6 mm to about 4.6 mm, about 2.6 mm to about 4.8 mm, about 2.6 mm to about 5.0 mm, about 2.7 mm to about 2.8 mm, about 2.7 mm to about 2.9 mm, about 2.7 mm to about 3.0 mm, about 2.7 mm to about 3.2 mm, about 2.7 mm to about 3.4 mm, about 2.7 mm to about 3.6 mm, about 2.7 mm to about 3.8 mm, about 2.7 mm to about 4.0 mm, about 2.7 mm to about 4.2 mm, about 2.7 mm to about 4.4 mm, about 2.7 mm to about 4.6 mm, about 2.7 mm to about 4.8 mm, about 2.7 mm to about 5.0 mm, about 2.8 mm to about 2.9 mm, about 2.8 mm to about 3.0 mm, about 2.8 mm to about 3.2 mm, about 2.8 mm to about 3.4 mm, about 2.8 mm to about 3.6 mm, about 2.8 mm to about 3.8 mm, about 2.8 mm to about 4.0 mm, about 2.8 mm to about 4.2 mm, about 2.8 mm to about 4.4 mm, about 2.8 mm to about 4.6 mm, about 2.8 mm to about 4.8 mm, about 2.8 mm to about 5.0 mm, about 2.9 mm to about 3.0 mm, about 2.9 mm to about 3.2 mm, about 2.9 mm to about 3.4 mm, about 2.9 mm to about 3.6 mm, about 2.9 mm to about 3.8 mm, about 2.9 mm to about 4.0 mm, about 2.9 mm to about 4.2 mm, about 2.9 mm to about 4.4 mm, about 2.9 mm to about 4.6 mm, about 2.9 mm to about 4.8 mm, about 2.9 mm to about 5.0 mm, about 3.0 mm to about 3.2 mm, about 3.0 mm to about 3.4 mm, about 3.0 mm to about 3.6 mm, about 3.0 mm to about 3.8 mm, about 3.0 mm to about 4.0 mm, about 3.0 mm to about 4.2 mm, about 3.0 mm to about 4.4 mm, about 3.0 mm to about 4.6 mm, about 3.0 mm to about 4.8 mm, about 3.0 mm to about 5.0 mm, about 3.2 mm to about 3.4 mm, about 3.2 mm to about 3.6 mm, about 3.2 mm to about 3.8 mm, about 3.2 mm to about 4.0 mm, about 3.2 mm to about 4.2 mm, about 3.2 mm to about 4.4 mm, about 3.2 mm to about 4.6 mm, about 3.2 mm to about 4.8 mm, about 3.2 mm to about 5.0 mm, about 3.4 mm to about 3.6 mm, about 3.4 mm to about 3.8 mm, about 3.4 mm to about 4.0 mm, about 3.4 mm to about 4.2 mm, about 3.4 mm to about 4.4 mm, about 3.4 mm to about 4.6 mm, about 3.4 mm to about 4.8 mm, about 3.4 mm to about 5.0 mm, about 3.6 mm to about 3.8 mm, about 3.6 mm to about 4.0 mm, about 3.6 mm to about 4.2 mm, about 3.6 mm to about 4.4 mm, about 3.6 mm to about 4.6 mm, about 3.6 mm to about 4.8 mm, about 3.6 mm to about 5.0 mm, about 3.8 mm to about 4.0 mm, about 3.8 mm to about 4.2 mm, about 3.8 mm to about 4.4 mm, about 3.8 mm to about 4.6 mm, about 3.8 mm to about 4.8 mm, about 3.8 mm to about 5.0 mm, about 4.0 mm to about 4.2 mm, about 4.0 mm to about 4.4 mm, about 4.0 mm to about 4.6 mm, about 4.0 mm to about 4.8 mm, about 4.0 mm to about 5.0 mm, about 4.2 mm to about 4.4 mm, about 4.2 mm to about 4.6 mm, about 4.2 mm to about 4.8 mm, about 4.2 mm to about 5.0 mm, about 4.4 mm to about 4.6 mm, about 4.4 mm to about 4.8 mm, about 4.4 mm to about 5.0 mm, about 4.6 mm to about 4.8 mm, about 4.6 mm to about 5.0 mm, or about 4.8 mm to about 5.0 mm.

Fundamentally, it will be appreciated that the primary lumen 50 of the exemplary embodiment of FIGS. 1 and 2 is defined by the cavity or space bound by the body wall 40 at the distal end 24 of the device 20, or the volume defined by the length or portion of the device 20 substantially from the distal shoulder 74 of the interior fitting 70 to the distal opening 34 of the body 30, representing the volume or space into which a food impaction or foreign body may be suctioned or captured and then withdrawn. Here, the secondary lumen 60 is defined by the cavity or space bound by the body wall 40 at the proximal end 22 of the device 20, or the annular proximal opening 32 of the body 30 into which the scope N (FIG. 6) is inserted. As such, in this illustrative embodiment, the primary and secondary lumens 50, 60 are not only contiguous, meaning connected or joined or in fluid communication, but are collinear or axially aligned, more about which is said below regarding the use illustrated in FIGS. 7A-7C. Those skilled in the art will appreciate that a variety of configurations and sizes of such lumens and walls beyond those shown and described and dimensionally characterized herein may be employed according to aspects of the present specification to suit particular clinical contexts, both in terms of treatment and the endoscope to be used, such that the foregoing dimensional call-outs are to be understood as illustrative and non-limiting.

Any such endoscope device 20 as shown and described herein may be single-use or multi-use and sold, supplied, or used “packaged clean” or “sterile” depending on the clinical context and applicable regulations in place at the time. The design and materials of construction of the device 20 contemplated herein, in whole or in part, do or can accommodate any such indications for use according to aspects of the present invention without departing from its spirit and scope.

Referring briefly now to FIG. 3, there is shown a side cross-sectional view of an alternative endoscope device 20, in accordance with at least one embodiment, substantially analogous to the embodiment of FIGS. 1 and 2, here with a body wall 40 having a taper toward the distal end 24 of the body 30, such that the distal opening 34 is slightly reduced in diameter relative to the central portion of the body 30. It will be appreciated that by tapering the distal end 24 of the body wall 40 and thereby slightly reducing the distal opening 34, the resulting truncated bullet-nosed or more ogive-shaped body 30 may further facilitate atraumatic intubation or passage into the anatomy as well as retention of any foreign material or food impaction being removed. While the tapered body wall 40 is shown as being accomplished through the shaping of the wall 40 while maintaining as substantially constant the wall thickness defined by the distance between the outer and inner surfaces 42, 44 of the wall 40, it will be appreciated that the taper can be accomplished by reducing the wall thickness toward the distal opening 34 and nose 46 or by some combination of shaping and wall thickness reduction. Accordingly, the tapered body wall 40 may take numerous forms beyond that shown and described according to aspects of the present specification without departing from its spirit and scope.

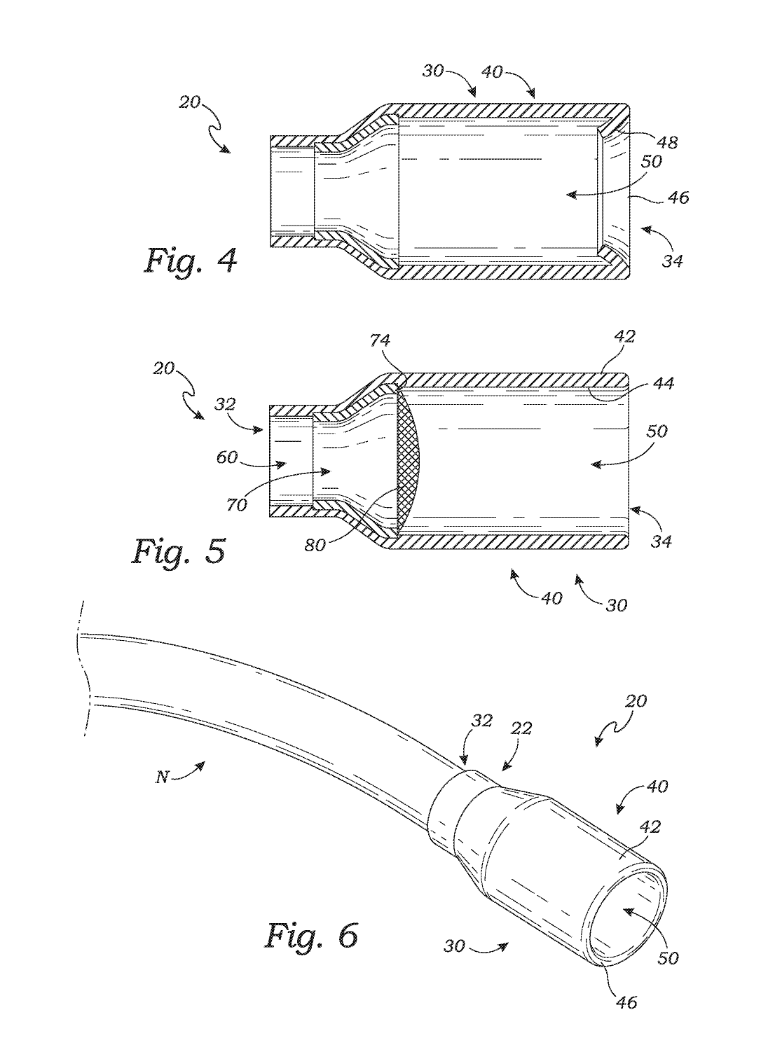

In FIG. 4, there is shown a side cross-sectional view of a further alternative endoscope device 20, in accordance with at least one embodiment, again substantially analogous to the embodiment of FIGS. 1 and 2, here with an inwardly and somewhat proximally extending lip 48 formed in the distal opening 34 of the device 20. In the exemplary embodiment, the lip 48 is at the distal opening 34 contiguous with the nose 46, but the lip 48 may be inset distally as well. Different proportional sizes and shapes and locations of the lip 48 beyond that illustrated in FIG. 4 are possible without departing from the spirit and scope of the invention. In any such embodiment, it will be appreciated that such a lip 48 may cooperate with the suction applied to and through the device 20 in use to guide into and retain within the primary lumen 50 any foreign object or food impaction that is to be retrieved, acting as an annular retaining barb. In that regard, the lip 48, though shown as integral with the body wall 40 and thus of a unitary material and even wall thickness, it will be further appreciated that the lip may be formed separately or of a different material with different properties or integrally with the body but at least having a reduced wall thickness. In any case, those skilled in the art will appreciate that the lip 48 may be formed by virtue of geometrical design and/or material to be somewhat resilient and biased inwardly so as to flex proximally and allow passage therethrough of any retrieved material while resisting flexing distally or toward the axial, so as to again cooperate with the suction or other tool implemented through any endoscope N (FIG. 6) being used in conjunction with the device 20 to secure within the primary lumen 50 and ultimately retrieve any undesired material from the anatomy such as the esophagus E (FIGS. 7A-7C). Accordingly, once more, the lip 48 may take numerous forms beyond that shown and described according to aspects of the present specification without departing from its spirit and scope.

Turning now to FIG. 5, there is shown another side cross-sectional view of a still further alternative endoscope device 20, in accordance with at least one embodiment, again substantially analogous to the embodiment of FIGS. 1 and 2, here with a mesh screen 80 or the like positioned across the proximal end of the primary lumen 50 so as to prevent clogging of the suction source such as an endoscope N (FIG. 6) inserted through the proximal opening 32 and positioned within the secondary lumen 60 during use. The screen 80 can take numerous forms and mesh sizes to suit particular applications such that the schematic illustration of FIG. 5 is to be understood as exemplary and non-limiting. In the embodiment shown wherein an interior fitting 70 is positioned within the body 30 toform a distal shoulder 74, it will be appreciated that the screen 80 may be mounted or installed on or adjacent to such distal shoulder 74 for mechanical support. Alternatively, whether or not a fitting 70 is incorporated, the screen may also be imbedded within or bonded or otherwise affixed to the inner surface 44 of the body wall 40 so as to be held in place. In a further embodiment, particularly where the endoscope device 20 is to be reusable, optionally the screen 80 may be removably installed within the body 30 for ease of cleaning. It will be appreciated that the screen 80 in any such arrangement is to be selected to allow for adequate air flow as suction is applied through the primary and secondary lumens 50, 60 while stopping any solid or semi-solid material that is being pulled into the distal primary lumen 50 and thereby preventing such material from impacting on clogging the suction port of the scope N. A further consideration is visualization by the endoscope N that in use with this embodiment is positioned within the secondary lumen 60 behind or proximal of the screen 80 and so must “look” through the screen 80 to “see” out the distal opening 34 of the device 20 and the anatomy or any impacted material beyond. Accounting for raster effects or graphics known in imaging and display technologies and the underlying principle that a single image (raster) is comprised of a multitude of pixels and characterized by the number of pixels or even bits per pixel, it will be appreciated that the bits or pixels visible to the optical instrumentation of the scope N, whether fiberoptic or CCD (charge coupled device), as through the openings in the screen 80, can be reconstructed by the scope N to create and send back the complete image or raster with little to no adverse impact on visualization or visual image clarity due to the screen 80. Similarly, based on screen size and configuration, the distance from the scope N to the screen, and the related image processing capabilities of the scope N, moire effects, or undesired image artifacts caused by secondary and visually evident superimposed patterns that are overlaid and spatially or rotationally displaced from one another, can be satisfactorily addressed even while including a screen 80 in the “line of sight” of the endoscope N. In one example, the finer the resolution of the imaging equipment or image sensor the finer mesh of the screen 80 without necessarily adversely affecting image quality or creating unwanted artifacts. Any such relationship is also a function of the distance from the imaging equipment (i.e., the lens) to the screen 80, with the closer distances further improving resolution or image processing relative to raster or moire effects that might otherwise be caused by the screen 80. Whereas, as the screen 80 is positioned further from the scope N, the mesh size of the screen 80 may need to be proportionally larger to still not have any adverse effects on image quality or visualization. In any case, it is unlikely that mesh sizes below 0.5 mm or 35 US standard mesh would be employed or required. But again, even when a finer mesh screen 80 is employed, sufficient visualization can still be accomplished based on a number of factors as set forth above. The screen 80 itself may be made of any suitable material and by any suitable process of manufacture now known or later developed and may be substantially convex as shown, substantially flat, or substantially concave depending on the application. The convex-shaped screen 80 may aid in directing suctioned debris toward the perimeter of the screen 80 so that image generated from the scope N may be less affected by blockage.

Referring now to FIG. 6, there is shown a partial perspective view of an endoscope N with an exemplary endoscope device 20, depicted in reduced scale relative to FIGS. 1-5, installed on the distal end thereof, in accordance with at least one embodiment. As seen, the endoscope N is simply inserted through the proximal opening 32 of the device 20 in a net- or interference- or press-fit frictional arrangement within the secondary lumen 60 (FIG. 2). As will be appreciated and was discussed above in connection with FIG. 2, the secondary lumen 60 is sized and configured to snugly fit on and about the distal end of the scope N so as to maintain the position of the device 20 on the scope N during use. Though not shown, internal ribs, knurling, or the like may be formed within the secondary lumen 60 to further facilitate the frictional engagement of the device 20 on or with the endoscope N. Once more, in the exemplary embodiment, the endoscope N can only be inserted so far distally within the secondary lumen 60 before it butts up against the proximal shoulder 72 formed within the body 30 of the device 20 by the interior fitting 70 (FIG. 2) so as to form a mechanical stop for the scope N and prevent its further distal advancement. Even with only a frictional engagement, the desired position of the device 20 on the scope N can be substantially maintained relative to the forces expected to be experienced by or applied to the device 20 during use, such as when being advanced within or withdrawn from the anatomy. Regardless, it is an objective to be able to transmit force to the device 20 as applied to the proximal end of an inserted endoscope N without the device 20 sliding further on or moving on the scope N. It will be appreciated that such objective can be accomplished in a variety of ways beyond those shown and described, such that the illustrated embodiments are to be understood as exemplary and non-limiting.

Turning now to FIGS. 7A-7C, there are shown schematic illustrations of an assembly of the exemplary device 20 on the distal end of an endoscope N as shown in FIG. 6 and now inserted within the esophagus E at various locations and in various modes of operation. First, as shown in FIG. 7A, the endoscope N and device 20 are passed through the mouth M and into the esophagus E of the patient H as by inserting the device 20 into the oral cavity 0 and following the hard palate down past the pharynx P and through the upper esophageal sphincter U in a manner generally known in the art for passing an endoscope or overtube into the esophagus E or stomach S. As shown, the device 20 and endoscope E are beginning their descent along the esophagus E toward a food impaction I located just above the lower esophageal sphincter L. Then, as shown in FIG. 7B, the endoscope N has been further advanced until the device 20 is positioned just above the food impaction I and thus readied for retrieval. Though not shown, it will be appreciated that the scope N and the device 20 could be advanced even further so as to directly contact and even press a bit into the impaction I potentially applying an increased suctional force upon the impaction I and further potentially creating a seal between the nose 46 and at least a portion of the impaction I. In any event, once the scope N and device 20 are so positioned, as shown in FIG. 7C, suction may then be applied from a suction source (not shown) as through the suction port of the scope N so as to draw the food impaction I into the primary lumen 50 (FIG. 2) of the device 20. Thereafter, with suction still applied, the endoscope N and device 20 may be withdrawn and the impaction I thereby removed from the patient H. In the illustrated embodiment, the impaction I shown is relatively small and so is retrieved in one pass of the scope N and device 20. In other clinical contexts, the food impaction or bolus may be significantly larger and so require several passes even with the increased volumetric capacity of the endoscope device 20 as compared with known devices. But fundamentally, an aspect and objective of the present invention is to remove a food impaction or other material in relatively fewer passes while under improved visualization. Certainly, as will be appreciated with reference to particularly FIG. 7B, it will be appreciated that the scope N inserted in the proximal end of the device 20 and able to “look” through the primary and secondary lumens 50, 60 (FIG. 2) is thus able to “see” the food impaction I or other material as suction commences and potentially throughout the suction event depending on the nature and amount of material being retrieved—this would be true regardless of the type of optical capability of the scope (e.g., whether fiberoptic or CCD) and of the kind of lens (e.g., straight, fish-eye, etc.). Relatedly, it is desired that, within anatomical limitations, the device 20 be as large as possible to retrieve as much material as possible per insertion while also not increasing the length so much so relative to the diameter or width of the device 20, and particularly the primary lumen 50 (FIG. 2), that the distal end or body wall 40 of the device 20 obstructs in whole or appreciable part the view from the scope N to the anatomy beyond the distal end 24 of the device 20, particularly during insertion. As one exemplary but non-limiting proportional arrangement, the length of the distal portion of the device 20 or effectively of the primary lumen 50 is approximately double the width in that same region, though again those skilled in the art will appreciate that other sizes or size ratios are possible. By way of further illustration, taking an example wherein an intermediate size of the device 20 is employed such as having a primary lumen length of approximately 40 mm and primary lumen width or inside diameter of approximately 20 mm, it will be appreciated that the approximate volume of the primary lumen 50 for the purpose of food impaction or other material retention is then approximately 12,500 mm³. By comparison, while in the illustrated embodiment of FIGS. 7A-7C the volume of food impaction is roughly equivalent, again thereby enabling its removal in a single pass, clinical cases have revealed food impactions on the order of one to one-and-a-half (1-1½) cups, with one cup being equivalent to approximately 236,000 mm³. As such, it will be appreciated that roughly 20 passes may be required to completely remove such a large impaction. While this seems significant, it is still greatly reduced from the number of passes that would be required employing known devices and techniques for non-surgical removal of such an impaction.

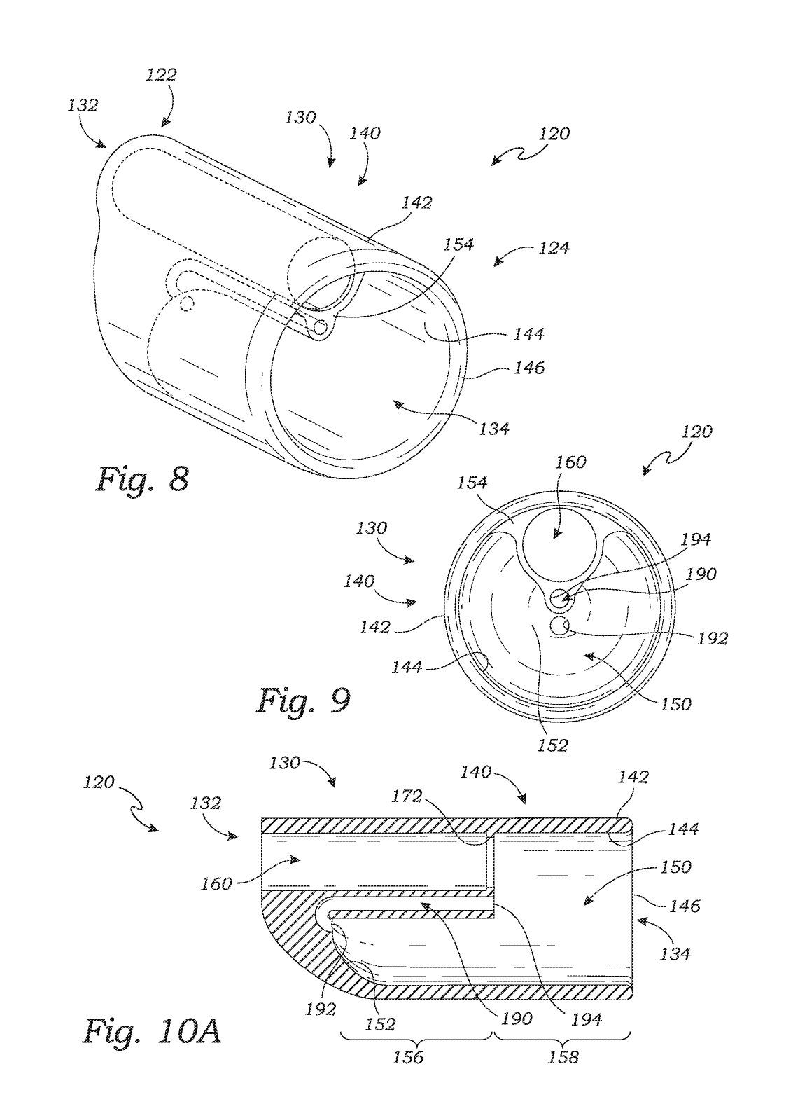

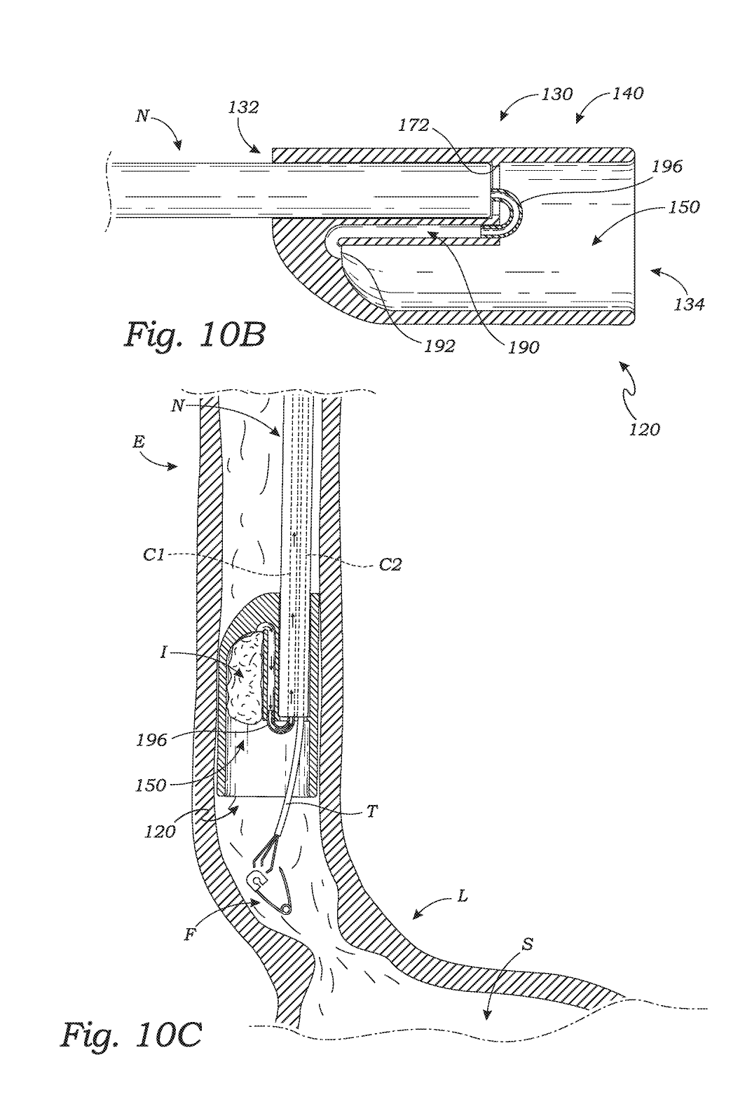

Referring now to FIGS. 8 and 9, there are shown a perspective view and an end view of an alternative exemplary endoscope device 120, in accordance with at least one embodiment, again configured for selective or removable attachment on the distal end of an endoscope N (FIG. 10B). The endoscope device 120 generally comprises a body 130 extending between a proximal end 122 and a distal end 124 and having a proximal opening 132 and an opposite distal opening 134, respectively. The body 130 is defined by a body wall 140 having an outer surface 142 and an opposite inner surface 144 with the distance between defining the effective wall thickness of the body wall 140. Within the body 130 of the endoscope device 120 there are formed a primary lumen 150 associated with suction or retrieval and a secondary lumen 160 associated with the endoscope N, more about which is said below. By way of overview, the primary lumen 150 is configured for suction to be selectively applied therethrough and is contiguous with the distal opening 134, while the secondary lumen 160 is formed within the body 130 so as to be in fluid communication with the primary lumen 150 and is contiguous with the proximal opening 132 and configured for frictional receipt of an endoscope N, whereby suction selectively applied through a suction port C1 of the endoscope N (FIG. 10C) when positioned in the secondary lumen 160 applies suction to the primary lumen 150.

More particularly, with continued reference to FIGS. 8 and 9 and further reference to the cross-sectional views of FIGS. 10A and 10B, as compared to the primary and secondary lumens 50, 60 being substantially coaxial as in the embodiment of the device 120 shown in FIGS. 1-7C, here the primary and secondary lumens 150, 160 are substantially offset, at least in part. Specifically, the secondary lumen 160 is shifted off-axis to one side of the body 130 and rather than being entirely proximal of the primary lumen 150 or extending distally to the far distal end 124 of the device 120, the secondary lumen 160 is essentially intermediate, ending at a distal wall 154 of the primary lumen 150 between the proximal and distal ends 122, 124 of the device 120. As such, as best seen in FIG. 10A, there is a proximal space 156 within the primary lumen 150 of the device 120 that substantially surrounds effectively on all but one side the distal portion of the secondary lumen 160, the proximal space 156 effectively defined or bounded at one end by the distal wall 154 that is common with the distal end of the secondary lumen 160 and at the other end by the floor or proximal wall 152 of the primary lumen 150. There is also a distal space 158 that is defined by the volume within the primary lumen 150 distal of the distal wall 154, or between the distal wall 154 and the distal opening 134 or nose 146 of the endoscope device 120. As such, it will be appreciated that there is effectively a volumetric space 158 within the primary lumen 150 that is distal or in front of the secondary lumen 160 and a space 156 within the primary lumen 150 that is proximal and essentially beside the secondary lumen 160. With continued reference to FIG. 10A, there is shown a suction channel 190 formed within the body 130 so as to communicate between the proximal and distal walls 152, 154 of the primary lumen 150, as by intersecting the proximal wall 152 so as to form a proximal channel opening 192 and intersecting the distal wall 154 so as to form a distal channel opening 194. Then, as shown in FIG. 10B, when an endoscope N is inserted through the proximal opening 132 and into the secondary lumen 160 of the device 120 for use, a suction tube 196 may have one end inserted within the suction port or channel C1 of the scope N and the other end inserted within the distal channel opening 194 of the suction channel 190 so as to form a fluid flow path from the suction port C1 of the scope N through the suction channel 190 to the proximal channel opening 192 formed in the proximal wall 152 or floor of the primary lumen 150. It will be appreciated that the suction applied through the suction port C1 of the endoscope N operably installed in the device 120 is thus effectively delivered to or acts at the bottom or proximal end of the primary lumen 150. In this way, any food impaction or foreign object pulled into the primary lumen 150 of the device 120 first flows or travels into the proximal space 156 or portion of the primary lumen 150.

Turning to FIG. 10C, there is shown a partial, reduced scale schematic view of the endoscope device 120 of FIG. 9 as installed on the distal end of an endoscope N, as shown in FIG. 10B, now inserted within the esophagus E to a position just above the lower esophageal sphincter L. As shown, with suction applied through the suction port C1 of the scope N and thereby at the proximal channel opening 192 through the suction tube 196 and suction channel 190 (FIG. 10B), as indicated by the arrows, a food impaction I is pulled into the primary lumen 150, and specifically within the proximal space 156 adjacent the secondary lumen 160 and thus the distal end of the endoscope N. Advantageously, any such material is thus retrieved by the endoscope device 120 in cooperation with the scope N without the impaction I actually blocking or being distal or in front of the scope 120. In this way, it will be appreciated that visualization throughout substantially the entire suction event is achieved, of both the impaction as it is pulled in and the anatomy before and after, thereby providing even better visual feedback and confirmation to the clinician performing the retrieval procedure. Moreover, as also shown in FIG. 10C, where the blockage comprises both food impaction I and a foreign object F, upon suction removal of what can be by that method, a tool T such as a grasper or forceps can be deployed from the scope N via a second channel C2 so as to grasp and retrieve such foreign object F as well, even in the same, single insertion of the scope N with device 120. As best seen in FIG. 10A, similar to other exemplary embodiments herein, a proximal or proximally-facing shoulder 172 may be formed at the distal end of the secondary lumen 160, in the vicinity of the distal wall 154 of the primary lumen 150 so as to provide a mechanical stop for the scope N and prevent its further distal insertion relative to the device 120; alternatively, the friction fit alone between the scope N and the secondary lumen 160 serves to retain the device 120 at the desired location relative to the scope N, even when force is applied. Those skilled in the art will appreciate that the proximal or recessed position (recessed relative to the nose 146) of the secondary lumen 160 and thus the distal end of the scope N relative to the distal end 124 of the device 120 effectively creates a distal volumetric space 158 maintained by the distal portion of the body wall 140 and within which secondary operations can be performed such as deploying a tool T for foreign body F retrieval or even taking a tissue sample, such as a polyp or other growth or abnormality on the esophagus E that may, in fact, be at least partly responsible for the impaction, all such secondary operations again being facilitated by unimpaired visualization and use of the distal end of the scope N and related deployable tools even while some amount of food impaction I has already been retrieved as enabled by the configuration of the device 120 as shown in FIG. 9. The primary lumen 150, and particularly the distal space 158 thereof, thus provides a region for endoscopic procedures and manipulations and safe removal of material such as food impaction I or particularly any foreign object F, such being shielded from the anatomy when pulled or positioned within the primary lumen 150. It will be further appreciated that additional functionality can be enabled by providing further channels (not shown) communicating between the proximal and distal walls 152, 154 of the primary lumen 150, for example, a second such channel being provided for irrigation or flushing of the primary lumen 150 as by connecting such channel to the irrigation port of the scope N in much the same way that the suction port is connected to the suction channel 190 using a suction tube 196. Alternatively, the irrigation port of the scope N may simply allow for flow into the distal space 158 of the primary lumen 150 to facilitate rinsing of the device 120 or wetting and suctioning of the impaction I. Finally, it will be appreciated that a screen (not shown) may be placed over the proximal channel opening 192 so as to prevent clogging of the suction channel 190 or tube 196 or the suction port of the scope N itself. Beneficially, such screen at the proximal end of the suction channel 190 and offset from the secondary lumen 160 and the scope N will thus in no way impair visualization through the scope N. It will be appreciated that a variety of other configurations of the device 120 beyond those shown and described wherein at least a portion of the secondary lumen 160 is offset from and at least partially surrounded by the primary lumen 150 are possible without departing from the spirit and scope of the present invention.

As shown, the device 120 may be generally proportioned so as to be longer than it is wide, and in one embodiment may be scaled such that its length is approximately double its width. In the alternative exemplary embodiment, the endoscope device 120 is again substantially annular, having a relatively larger diameter at its distal end 124 associated with the distal opening 134 through which a food impaction or other material may be suctioned within the device 120, in whole or in part, and a relatively smaller diameter at its proximal end 122 associated with a proximal opening 132 through which the distal end of the endoscope N is inserted when the device 120 is in use. Those skilled in the art should again understand that all figures are schematic in nature and fundamentally that none of the figures are to be taken literally or to scale, each such figure being simply illustrative of features and aspects of the present invention and non-limiting. Thus, while the exemplary device 120 is shown as being annular, this is not necessarily the case, as other geometries such as elliptical may also be employed in at least portions of the endoscope device 120. Once more, where the device 120 or any feature thereof is annular or round, it will be appreciated that any reference to “width” is to be understood as the feature's diameter. Dimensionally, the overall length and width of the alternative exemplary device 120 of FIGS. 8-10C is comparable to those of the first exemplary device 20 of FIGS. 1-7C, as are the general sizes of the primary and secondary lumens 150, 160 and the resulting volumes, with the secondary lumen 160 of the alternative embodiment being proportionally longer than that of the first exemplary embodiment. In the alternative embodiment, as noted above, the primary lumen 150, while potentially being substantially similar in overall volume to that of the first exemplary embodiment, is nonetheless divided into two distinct spaces—a distal space 158 that is distal and in front of the secondary lumen 160 and a proximal space 156 that is substantially adjacent to the secondary lumen 158, which spaces 156, 158 bear further discussion in terms of their dimensional characteristics.

Fundamentally, the offset or recessing of the distal wall 154 of the primary lumen 150 back from the nose 146 effectively sets the sizes of the proximal and distal spaces 156, 158 comprising the primary lumen 150. In the exemplary embodiment, such offset of the distal wall 154 to the nose 146 is between 1 and 40 mm, with it being appreciated that as the offset gets smaller (i.e., toward 1 mm) the proximal space 156 gets proportionately larger and the distal space 158 gets proportionately smaller and as the offset gets larger (i.e., toward 40 mm) the proximal space 156 gets proportionately smaller and the distal space 158 gets proportionately larger. In different contexts and intended uses, one configuration versus another of the primary lumen 150 and thus the endoscope device 120 may be preferable.

Therefore, in one exemplary embodiment, the offset of the distal wall 154 of the primary lumen 150 from the nose 146 is, e.g., about 1 mm, about 2 mm, about 4 mm, about 6 mm, about 8 mm, about 10 mm, about 12 mm, about 14 mm, about 16 mm, about 18 mm, about 20 mm, about 22 mm, about 24 mm, about 26 mm, about 28 mm, about 30 mm, about 32 mm, about 34 mm, about 36 mm, about 38 mm, or about 40 mm. In other aspects of this embodiment, in an endoscope device 120 disclosed herein the offset of the distal wall 154 of the primary lumen 150 from the nose 146 is, e.g., at least 1 mm, at least 2 mm, at least 4 mm, at least 6 mm, at least 8 mm, at least 10 mm, at least 12 mm, at least 14 mm, at least 16 mm, at least 18 mm, at least 20 mm, at least 22 mm, at least 24 mm, at least 26 mm, at least 28 mm, at least 30 mm, at least 32 mm, at least 34 mm, at least 36 mm, at least 38 mm, or at least 40 mm. In still other aspects of this embodiment, in an endoscope device 120 disclosed herein the offset of the distal wall 154 of the primary lumen 150 from the nose 146 is, e.g., at most 1 mm, at most 2 mm, at most 4 mm, at most 6 mm, at most 8 mm, at most 10 mm, at most 12 mm, at most 14 mm, at most 16 mm, at most 18 mm, at most 20 mm, at most 22 mm, at most 24 mm, at most 26 mm, at most 28 mm, at most 30 mm, at most 32 mm, at most 34 mm, at most 36 mm, at most 38 mm, or at most 40 mm.

In further aspects of this embodiment, the offset of the distal wall 154 of the primary lumen 150 from the nose 146 is, e.g., about 1 mm to about 2 mm, about 1 mm to about 4 mm, about 1 mm to about 6 mm, about 1 mm to about 8 mm, about 1 mm to about 10 mm, about 1 mm to about 12 mm, about 1 mm to about 14 mm, about 1 mm to about 16 mm, about 1 mm to about 18 mm, about 1 mm to about 20 mm, about 1 mm to about 22 mm, about 1 mm to about 24 mm, about 1 mm to about 26 mm, about 1 mm to about 28 mm, about 1 mm to about 30 mm, about 1 mm to about 32 mm, about 1 mm to about 34 mm, about 1 mm to about 36 mm, about 1 mm to about 38 mm, about 1 mm to about 40 mm, about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 2 mm to about 22 mm, about 2 mm to about 24 mm, about 2 mm to about 26 mm, about 2 mm to about 28 mm, about 2 mm to about 30 mm, about 2 mm to about 32 mm, about 2 mm to about 34 mm, about 2 mm to about 36 mm, about 2 mm to about 38 mm, about 2 mm to about 40 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 4 mm to about 22 mm, about 4 mm to about 24 mm, about 4 mm to about 26 mm, about 4 mm to about 28 mm, about 4 mm to about 30 mm, about 4 mm to about 32 mm, about 4 mm to about 34 mm, about 4 mm to about 36 mm, about 4 mm to about 38 mm, about 4 mm to about 40 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 6 mm to about 22 mm, about 6 mm to about 24 mm, about 6 mm to about 26 mm, about 6 mm to about 28 mm, about 6 mm to about 30 mm, about 6 mm to about 32 mm, about 6 mm to about 34 mm, about 6 mm to about 36 mm, about 6 mm to about 38 mm, about 6 mm to about 40 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 8 mm to about 22 mm, about 8 mm to about 24 mm, about 8 mm to about 26 mm, about 8 mm to about 28 mm, about 8 mm to about 30 mm, about 8 mm to about 32 mm, about 8 mm to about 34 mm, about 8 mm to about 36 mm, about 8 mm to about 38 mm, about 8 mm to about 40 mm, about 10 mm to about 12 mm, about 10 mm to about 14 mm, about 10 mm to about 16 mm, about 10 mm to about 18 mm, about 10 mm to about 20 mm, about 10 mm to about 22 mm, about 10 mm to about 24 mm, about 10 mm to about 26 mm, about 10 mm to about 28 mm, about 10 mm to about 30 mm, about 10 mm to about 32 mm, about 10 mm to about 34 mm, about 10 mm to about 36 mm, about 10 mm to about 38 mm, about 10 mm to about 40 mm, about 12 mm to about 14 mm, about 12 mm to about 16 mm, about 12 mm to about 18 mm, about 12 mm to about 20 mm, about 12 mm to about 22 mm, about 12 mm to about 24 mm, about 12 mm to about 26 mm, about 12 mm to about 28 mm, about 12 mm to about 30 mm, about 12 mm to about 32 mm, about 12 mm to about 34 mm, about 12 mm to about 36 mm, about 12 mm to about 38 mm, about 12 mm to about 40 mm, about 14 mm to about 16 mm, about 14 mm to about 18 mm, about 14 mm to about 20 mm, about 14 mm to about 22 mm, about 14 mm to about 24 mm, about 14 mm to about 26 mm, about 14 mm to about 28 mm, about 14 mm to about 30 mm, about 14 mm to about 32 mm, about 14 mm to about 34 mm, about 14 mm to about 36 mm, about 14 mm to about 38 mm, about 14 mm to about 40 mm, about 16 mm to about 18 mm, about 16 mm to about 20 mm, about 16 mm to about 22 mm, about 16 mm to about 24 mm, about 16 mm to about 26 mm, about 16 mm to about 28 mm, about 16 mm to about 30 mm, about 16 mm to about 32 mm, about 16 mm to about 34 mm, about 16 mm to about 36 mm, about 16 mm to about 38 mm, about 16 mm to about 40 mm, about 18 mm to about 20 mm, about 18 mm to about 22 mm, about 18 mm to about 24 mm, about 18 mm to about 26 mm, about 18 mm to about 28 mm, about 18 mm to about 30 mm, about 18 mm to about 32 mm, about 18 mm to about 34 mm, about 18 mm to about 36 mm, about 18 mm to about 38 mm, about 18 mm to about 40 mm, about 20 mm to about 22 mm, about 20 mm to about 24 mm, about 20 mm to about 26 mm, about 20 mm to about 28 mm, about 20 mm to about 30 mm, about 20 mm to about 32 mm, about 20 mm to about 34 mm, about 20 mm to about 36 mm, about 20 mm to about 38 mm, about 20 mm to about 40 mm, about 22 mm to about 24 mm, about 22 mm to about 26 mm, about 22 mm to about 28 mm, about 22 mm to about 30 mm, about 22 mm to about 32 mm, about 22 mm to about 34 mm, about 22 mm to about 36 mm, about 22 mm to about 38 mm, about 22 mm to about 40 mm, about 24 mm to about 26 mm, about 24 mm to about 28 mm, about 24 mm to about 30 mm, about 24 mm to about 32 mm, about 24 mm to about 34 mm, about 24 mm to about 36 mm, about 24 mm to about 38 mm, about 24 mm to about 40 mm, about 26 mm to about 28 mm, about 26 mm to about 30 mm, about 26 mm to about 32 mm, about 26 mm to about 34 mm, about 26 mm to about 36 mm, about 26 mm to about 38 mm, about 26 mm to about 40 mm, about 28 mm to about 30 mm, about 28 mm to about 32 mm, about 28 mm to about 34 mm, about 28 mm to about 36 mm, about 28 mm to about 38 mm, about 28 mm to about 40 mm, about 30 mm to about 32 mm, about 30 mm to about 34 mm, about 30 mm to about 36 mm, about 30 mm to about 38 mm, about 30 mm to about 40 mm, about 32 mm to about 34 mm, about 32 mm to about 36 mm, about 32 mm to about 38 mm, about 32 mm to about 40 mm, about 34 mm to about 36 mm, about 34 mm to about 38 mm, about 34 mm to about 40 mm, about 36 mm to about 38 mm, about 36 mm to about 40 mm, or about 38 mm to about 40 mm.

In terms of the volume of the primary lumen spaces 156, 158, taking for example the overall volume of the primary lumen 150 in an endoscope device 120 disclosed herein to be on the order of about 1,700 mm³ to about 100,000 mm³, the proximal space 156 may have a volume of between 750 mm³ and 90,000 mm³, again depending on the offset position of the distal wall 154 of the primary lumen 150. Thus, the volume of the proximal space 156 is, e.g., about 750 mm³, about 1,000 mm³, about 1,100 mm³, about 1,200 mm³, about 1,300 mm³, about 1,400 mm³, about 1,500 mm³, about 1,600 mm³, about 1,700 mm³, about 1,800 mm³, about 1,900 mm³, about 2,000 mm³, about 2,200 mm³, about 2,400 mm³, about 2,600 mm³, about 2,800 mm³, about 3,000 mm³, about 4,000 mm³, about 5,000 mm³, about 6,000 mm³, about 7,000 mm³, about 8,000 mm³, about 9,000 mm³, about 10,000 mm³, about 12,000 mm³, about 14,000 mm³, about 16,000 mm³, about 18,000 mm³, about 20,000 mm³, about 25,000 mm³, about 30,000 mm³, about 35,000 mm³, about 40,000 mm³, about 45,000 mm³, about 50,000 mm³, about 60,000 mm³, about 70,000 mm³, about 80,000 mm³, or about 90,000 mm³. In still further aspects of this embodiment, the volume of the proximal space 156 is, e.g., at least 750 mm³, at least 1,000 mm³, at least 1,100 mm³, at least 1,200 mm³, at least 1,300 mm³, at least 1,400 mm³, at least 1,500 mm³, at least 1,600 mm³, at least 1,700 mm³, at least 1,800 mm³, at least 1,900 mm³, at least 2,000 mm³, at least 2,200 mm³, at least 2,400 mm³, at least 2,600 mm³, at least 2,800 mm³, at least 3,000 mm³, at least 4,000 mm³, at least 5,000 mm³, at least 6,000 mm³, at least 7,000 mm³, at least 8,000 mm³, at least 9,000 mm³, at least 10,000 mm³, at least 12,000 mm³, at least 14,000 mm³, at least 16,000 mm³, at least 18,000 mm³, at least 20,000 mm³, at least 25,000 mm³, at least 30,000 mm³, at least 35,000 mm³, at least 40,000 mm³, at least 45,000 mm³, at least 50,000 mm³, at least 60,000 mm³, at least 70,000 mm³, at least 80,000 mm³, or at least 90,000 mm³. In still further aspects of this embodiment, the volume of the proximal space 156 is, e.g., at most 750 mm³, at most 1,000 mm³, at most 1,100 mm³, at most 1,200 mm³, at most 1,300 mm³, at most 1,400 mm³, at most 1,500 mm³, at most 1,600 mm³, at most 1,700 mm³, at most 1,800 mm³, at most 1,900 mm³, at most 2,000 mm³, at most 2,200 mm³, at most 2,400 mm³, at most 2,600 mm³, at most 2,800 mm³, at most 3,000 mm³, at most 4,000 mm³, at most 5,000 mm³, at most 6,000 mm³, at most 7,000 mm³, at most 8,000 mm³, at most 9,000 mm³, at most 10,000 mm³, at most 12,000 mm³, at most 14,000 mm³, at most 16,000 mm³, at most 18,000 mm³, at most 20,000 mm³, at most 25,000 mm³, at most 30,000 mm³, at most 35,000 mm³, at most 40,000 mm³, at most 45,000 mm³, at most 50,000 mm³, at most 60,000 mm³, at most 70,000 mm³, at most 80,000 mm³, or at most 90,000 mm³.

In other aspects of this embodiment, in an endoscope device 120 disclosed herein the volume of the proximal space 156 is, e.g., about 750 mm³ to about 1,000 mm³, about 750 mm³ to about 1,100 mm³, about 750 mm³ to about 1,200 mm³, about 750 mm³ to about 1,300 mm³, about 750 mm³ to about 1,400 mm³, about 750 mm³ to about 1,500 mm³, about 750 mm³ to about 1,600 mm³, about 750 mm³ to about 1,700 mm³, about 750 mm³ to about 1,800 mm³, about 750 mm³ to about 1,900 mm³, about 750 mm³ to about 2,000 mm³, about 750 mm³ to about 2,200 mm³, about 750 mm³ to about 2,400 mm³, about 750 mm³ to about 2,600 mm³, about 750 mm³ to about 2,800 mm³, about 750 mm³ to about 3,000 mm³, about 750 mm³ to about 4,000 mm³, about 750 mm³ to about 5,000 mm³, about 750 mm³ to about 6,000 mm³, about 750 mm³ to about 7,000 mm³, about 750 mm³ to about 8,000 mm³, about 750 mm³ to about 9,000 mm³, about 750 mm³ to about 10,000 mm³, about 750 mm³ to about 12,000 mm³, about 750 mm³ to about 14,000 mm³, about 750 mm³ to about 16,000 mm³, about 750 mm³ to about 18,000 mm³, about 750 mm³ to about 20,000 mm³, about 750 mm³ to about 25,000 mm³, about 750 mm³ to about 30,000 mm³, about 750 mm³ to about 35,000 mm³, about 750 mm³ to about 40,000 mm³, about 750 mm³ to about 45,000 mm³, about 750 mm³ to about 50,000 mm³, about 750 mm³ to about 60,000 mm³, about 750 mm³ to about 70,000 mm³, about 750 mm³ to about 80,000 mm³, about 750 mm³ to about 90,000 mm³, about 1,000 mm³ to about 1,100 mm³, about 1,000 mm³ to about 1,200 mm³, about 1,000 mm³ to about 1,300 mm³, about 1,000 mm³ to about 1,400 mm³, about 1,000 mm³ to about 1,500 mm³, about 1,000 mm³ to about 1,600 mm³, about 1,000 mm³ to about 1,700 mm³, about 1,000 mm³ to about 1,800 mm³, about 1,000 mm³ to about 1,900 mm³, about 1,000 mm³ to about 2,000 mm³, about 1,000 mm³ to about 2,200 mm³, about 1,000 mm³ to about 2,400 mm³, about 1,000 mm³ to about 2,600 mm³, about 1,000 mm³ to about 2,800 mm³, about 1,000 mm³ to about 3,000 mm³, about 1,000 mm³ to about 4,000 mm³, about 1,000 mm³ to about 5,000 mm³, about 1,000 mm³ to about 6,000 mm³, about 1,000 mm³ to about 7,000 mm³, about 1,000 mm³ to about 8,000 mm³, about 1,000 mm³ to about 9,000 mm³, about 1,000 mm³ to about 10,000 mm³, about 1,000 mm³ to about 12,000 mm³, about 1,000 mm³ to about 14,000 mm³, about 1,000 mm³ to about 16,000 mm³, about 1,000 mm³ to about 18,000 mm³, about 1,000 mm³ to about 20,000 mm³, about 1,000 mm³ to about 25,000 mm³, about 1,000 mm³ to about 30,000 mm³, about 1,000 mm³ to about 35,000 mm³, about 1,000 mm³ to about 40,000 mm³, about 1,000 mm³ to about 45,000 mm³, about 1,000 mm³ to about 50,000 mm³, about 1,000 mm³ to about 60,000 mm³, about 1,000 mm³ to about 70,000 mm³, about 1,000 mm³ to about 80,000 mm³, about 1,000 mm³ to about 90,000 mm³, about 1,100 mm³ to about 1,200 mm³, about 1,100 mm³ to about 1,300 mm³, about 1,100 mm³ to about 1,400 mm³, about 1,100 mm³ to about 1,500 mm³, about 1,100 mm³ to about 1,600 mm³, about 1,100 mm³ to about 1,700 mm³, about 1,100 mm³ to about 1,800 mm³, about 1,100 mm³ to about 1,900 mm³, about 1,100 mm³ to about 2,000 mm³, about 1,100 mm³ to about 2,200 mm³, about 1,100 mm³ to about 2,400 mm³, about 1,100 mm³ to about 2,600 mm³, about 1,100 mm³ to about 2,800 mm³, about 1,100 mm³ to about 3,000 mm³, about 1,100 mm³ to about 4,000 mm³, about 1,100 mm³ to about 5,000 mm³, about 1,100 mm³ to about 6,000 mm³, about 1,100 mm³ to about 7,000 mm³, about 1,100 mm³ to about 8,000 mm³, about 1,100 mm³ to about 9,000 mm³, about 1,100 mm³ to about 10,000 mm³, about 1,100 mm³ to about 12,000 mm³, about 1,100 mm³ to about 14,000 mm³, about 1,100 mm³ to about 16,000 mm³, about 1,100 mm³ to about 18,000 mm³, about 1,100 mm³ to about 20,000 mm³, about 1,100 mm³ to about 25,000 mm³, about 1,100 mm³ to about 30,000 mm³, about 1,100 mm³ to about 35,000 mm³, about 1,100 mm³ to about 40,000 mm³, about 1,100 mm³ to about 45,000 mm³, about 1,100 mm³ to about 50,000 mm³, about 1,100 mm³ to about 60,000 mm³, about 1,100 mm³ to about 70,000 mm³, about 1,100 mm³ to about 80,000 mm³, about 1,100 mm³ to about 90,000 mm³, about 1,200 mm³ to about 1,300 mm³, about 1,200 mm³ to about 1,400 mm³, about 1,200 mm³ to about 1,500 mm³, about 1,200 mm³ to about 1,600 mm³, about 1,200 mm³ to about 1,700 mm³, about 1,200 mm³ to about 1,800 mm³, about 1,200 mm³ to about 1,900 mm³, about 1,200 mm³ to about 2,000 mm³, about 1,200 mm³ to about 2,200 mm³, about 1,200 mm³ to about 2,400 mm³, about 1,200 mm³ to about 2,600 mm³, about 1,200 mm³ to about 2,800 mm³, about 1,200 mm³ to about 3,000 mm³, about 1,200 mm³ to about 4,000 mm³, about 1,200 mm³ to about 5,000 mm³, about 1,200 mm³ to about 6,000 mm³, about 1,200 mm³ to about 7,000 mm³, about 1,200 mm³ to about 8,000 mm³, about 1,200 mm³ to about 9,000 mm³, about 1,200 mm³ to about 10,000 mm³, about 1,200 mm³ to about 12,000 mm³, about 1,200 mm³ to about 14,000 mm³, about 1,200 mm³ to about 16,000 mm³, about 1,200 mm³ to about 18,000 mm³, about 1,200 mm³ to about 20,000 mm³, about 1,200 mm³ to about 25,000 mm³, about 1,200 mm³ to about 30,000 mm³, about 1,200 mm³ to about 35,000 mm³, about 1,200 mm³ to about 40,000 mm³, about 1,200 mm³ to about 45,000 mm³, about 1,200 mm³ to about 50,000 mm³, about 1,200 mm³ to about 60,000 mm³, about 1,200 mm³ to about 70,000 mm³, about 1,200 mm³ to about 80,000 mm³, about 1,200 mm³ to about 90,000 mm³, about 1,300 mm³ to about 1,400 mm³, about 1,300 mm³ to about 1,500 mm³, about 1,300 mm³ to about 1,600 mm³, about 1,300 mm³ to about 1,700 mm³, about 1,300 mm³ to about 1,800 mm³, about 1,300 mm³ to about 1,900 mm³, about 1,300 mm³ to about 2,000 mm³, about 1,300 mm³ to about 2,200 mm³, about 1,300 mm³ to about 2,400 mm³, about 1,300 mm³ to about 2,600 mm³, about 1,300 mm³ to about 2,800 mm³, about 1,300 mm³ to about 3,000 mm³, about 1,300 mm³ to about 4,000 mm³, about 1,300 mm³ to about 5,000 mm³, about 1,300 mm³ to about 6,000 mm³, about 1,300 mm³ to about 7,000 mm³, about 1,300 mm³ to about 8,000 mm³, about 1,300 mm³ to about 9,000 mm³, about 1,300 mm³ to about 10,000 mm³, about 1,300 mm³ to about 12,000 mm³, about 1,300 mm³ to about 14,000 mm³, about 1,300 mm³ to about 16,000 mm³, about 1,300 mm³ to about 18,000 mm³, about 1,300 mm³ to about 20,000 mm³, about 1,300 mm³ to about 25,000 mm³, about 1,300 mm³ to about 30,000 mm³, about 1,300 mm³ to about 35,000 mm³, about 1,300 mm³ to about 40,000 mm³, about 1,300 mm³ to about 45,000 mm³, about 1,300 mm³ to about 50,000 mm³, about 1,300 mm³ to about 60,000 mm³, about 1,300 mm³ to about 70,000 mm³, about 1,300 mm³ to about 80,000 mm³, about 1,300 mm³ to about 90,000 mm³, about 1,400 mm³ to about 1,500 mm³, about 1,400 mm³ to about 1,600 mm³, about 1,400 mm³ to about 1,700 mm³, about 1,400 mm³ to about 1,800 mm³, about 1,400 mm³ to about 1,900 mm³, about 1,400 mm³ to about 2,000 mm³, about 1,400 mm³ to about 2,200 mm³, about 1,400 mm³ to about 2,400 mm³, about 1,400 mm³ to about 2,600 mm³, about 1,400 mm³ to about 2,800 mm³, about 1,400 mm³ to about 3,000 mm³, about 1,400 mm³ to about 4,000 mm³, about 1,400 mm³ to about 5,000 mm³, about 1,400 mm³ to about 6,000 mm³, about 1,400 mm³ to about 7,000 mm³, about 1,400 mm³ to about 8,000 mm³, about 1,400 mm³ to about 9,000 mm³, about 1,400 mm³ to about 10,000 mm³, about 1,400 mm³ to about 12,000 mm³, about 1,400 mm³ to about 14,000 mm³, about 1,400 mm³ to about 16,000 mm³, about 1,400 mm³ to about 18,000 mm³, about 1,400 mm³ to about 20,000 mm³, about 1,400 mm³ to about 25,000 mm³, about 1,400 mm³ to about 30,000 mm³, about 1,400 mm³ to about 35,000 mm³, about 1,400 mm³ to about 40,000 mm³, about 1,400 mm³ to about 45,000 mm³, about 1,400 mm³ to about 50,000 mm³, about 1,400 mm³ to about 60,000 mm³, about 1,400 mm³ to about 70,000 mm³, about 1,400 mm³ to about 80,000 mm³, about 1,400 mm³ to about 90,000 mm³, about 1,500 mm³ to about 1,600 mm³, about 1,500 mm³ to about 1,700 mm³, about 1,500 mm³ to about 1,800 mm³, about 1,500 mm³ to about 1,900 mm³, about 1,500 mm³ to about 2,000 mm³, about 1,500 mm³ to about 2,200 mm³, about 1,500 mm³ to about 2,400 mm³, about 1,500 mm³ to about 2,600 mm³, about 1,500 mm³ to about 2,800 mm³, about 1,500 mm³ to about 3,000 mm³, about 1,500 mm³ to about 4,000 mm³, about 1,500 mm³ to about 5,000 mm³, about 1,500 mm³ to about 6,000 mm³, about 1,500 mm³ to about 7,000 mm³, about 1,500 mm³ to about 8,000 mm³, about 1,500 mm³ to about 9,000 mm³, about 1,500 mm³ to about 10,000 mm³, about 1,500 mm³ to about 12,000 mm³, about 1,500 mm³ to about 14,000 mm³, about 1,500 mm³ to about 16,000 mm³, about 1,500 mm³ to about 18,000 mm³, about 1,500 mm³ to about 20,000 mm³, about 1,500 mm³ to about 25,000 mm³, about 1,500 mm³ to about 30,000 mm³, about 1,500 mm³ to about 35,000 mm³, about 1,500 mm³ to about 40,000 mm³, about 1,500 mm³ to about 45,000 mm³, about 1,500 mm³ to about 50,000 mm³, about 1,500 mm³ to about 60,000 mm³, about 1,500 mm³ to about 70,000 mm³, about 1,500 mm³ to about 80,000 mm³, about 1,500 mm³ to about 90,000 mm³, about 1,600 mm³ to about 1,700 mm³, about 1,600 mm³ to about 1,800 mm³, about 1,600 mm³ to about 1,900 mm³, about 1,600 mm³ to about 2,000 mm³, about 1,600 mm³ to about 2,200 mm³, about 1,600 mm³ to about 2,400 mm³, about 1,600 mm³ to about 2,600 mm³, about 1,600 mm³ to about 2,800 mm³, about 1,600 mm³ to about 3,000 mm³, about 1,600 mm³ to about 4,000 mm³, about 1,600 mm³ to about 5,000 mm³, about 1,600 mm³ to about 6,000 mm³, about 1,600 mm³ to about 7,000 mm³, about 1,600 mm³ to about 8,000 mm³, about 1,600 mm³ to about 9,000 mm³, about 1,600 mm³ to about 10,000 mm³, about 1,600 mm³ to about 12,000 mm³, about 1,600 mm³ to about 14,000 mm³, about 1,600 mm³ to about 16,000 mm³, about 1,600 mm³ to about 18,000 mm³, about 1,600 mm³ to about 20,000 mm³, about 1,600 mm³ to about 25,000 mm³, about 1,600 mm³ to about 30,000 mm³, about 1,600 mm³ to about 35,000 mm³, about 1,600 mm³ to about 40,000 mm³, about 1,600 mm³ to about 45,000 mm³, about 1,600 mm³ to about 50,000 mm³, about 1,600 mm³ to about 60,000 mm³, about 1,600 mm³ to about 70,000 mm³, about 1,600 mm³ to about 80,000 mm³, about 1,600 mm³ to about 90,000 mm³, about 1,700 mm³ to about 1,800 mm³, about 1,700 mm³ to about 1,900 mm³, about 1,700 mm³ to about 2,000 mm³, about 1,700 mm³ to about 2,200 mm³, about 1,700 mm³ to about 2,400 mm³, about 1,700 mm³ to about 2,600 mm³, about 1,700 mm³ to about 2,800 mm³, about 1,700 mm³ to about 3,000 mm³, about 1,700 mm³ to about 4,000 mm³, about 1,700 mm³ to about 5,000 mm³, about 1,700 mm³ to about 6,000 mm³, about 1,700 mm³ to about 7,000 mm³, about 1,700 mm³ to about 8,000 mm³, about 1,700 mm³ to about 9,000 mm³, about 1,700 mm³ to about 10,000 mm³, about 1,700 mm³ to about 12,000 mm³, about 1,700 mm³ to about 14,000 mm³, about 1,700 mm³ to about 16,000 mm³, about 1,700 mm³ to about 18,000 mm³, about 1,700 mm³ to about 20,000 mm³, about 1,700 mm³ to about 25,000 mm³, about 1,700 mm³ to about 30,000 mm³, about 1,700 mm³ to about 35,000 mm³, about 1,700 mm³ to about 40,000 mm³, about 1,700 mm³ to about 45,000 mm³, about 1,700 mm³ to about 50,000 mm³, about 1,700 mm³ to about 60,000 mm³, about 1,700 mm³ to about 70,000 mm³, about 1,700 mm³ to about 80,000 mm³, about 1,700 mm³ to about 90,000 mm³, about 1,800 mm³ to about 1,900 mm³, about 1,800 mm³ to about 2,000 mm³, about 1,800 mm³ to about 2,200 mm³, about 1,800 mm³ to about 2,400 mm³, about 1,800 mm³ to about 2,600 mm³, about 1,800 mm³ to about 2,800 mm³, about 1,800 mm³ to about 3,000 mm³, about 1,800 mm³ to about 4,000 mm³, about 1,800 mm³ to about 5,000 mm³, about 1,800 mm³ to about 6,000 mm³, about 1,800 mm³ to about 7,000 mm³, about 1,800 mm³ to about 8,000 mm³, about 1,800 mm³ to about 9,000 mm³, about 1,800 mm³ to about 10,000 mm³, about 1,800 mm³ to about 12,000 mm³, about 1,800 mm³ to about 14,000 mm³, about 1,800 mm³ to about 16,000 mm³, about 1,800 mm³ to about 18,000 mm³, about 1,800 mm³ to about 20,000 mm³, about 1,800 mm³ to about 25,000 mm³, about 1,800 mm³ to about 30,000 mm³, about 1,800 mm³ to about 35,000 mm³, about 1,800 mm³ to about 40,000 mm³, about 1,800 mm³ to about 45,000 mm³, about 1,800 mm³ to about 50,000 mm³, about 1,800 mm³ to about 60,000 mm³, about 1,800 mm³ to about 70,000 mm³, about 1,800 mm³ to about 80,000 mm³, about 1,800 mm³ to about 90,000 mm³, about 1,900 mm³ to about 2,000 mm³, about 1,900 mm³ to about 2,200 mm³, about 1,900 mm³ to about 2,400 mm³, about 1,900 mm³ to about 2,600 mm³, about 1,900 mm³ to about 2,800 mm³, about 1,900 mm³ to about 3,000 mm³, about 1,900 mm³ to about 4,000 mm³, about 1,900 mm³ to about 5,000 mm³, about 1,900 mm³ to about 6,000 mm³, about 1,900 mm³ to about 7,000 mm³, about 1,900 mm³ to about 8,000 mm³, about 1,900 mm³ to about 9,000 mm³, about 1,900 mm³ to about 10,000 mm³, about 1,900 mm³ to about 12,000 mm³, about 1,900 mm³ to about 14,000 mm³, about 1,900 mm³ to about 16,000 mm³, about 1,900 mm³ to about 18,000 mm³, about 1,900 mm³ to about 20,000 mm³, about 1,900 mm³ to about 25,000 mm³, about 1,900 mm³ to about 30,000 mm³, about 1,900 mm³ to about 35,000 mm³, about 1,900 mm³ to about 40,000 mm³, about 1,900 mm³ to about 45,000 mm³, about 1,900 mm³ to about 50,000 mm³, about 1,900 mm³ to about 60,000 mm³, about 1,900 mm³ to about 70,000 mm³, about 1,900 mm³ to about 80,000 mm³, about 1,900 mm³ to about 90,000 mm³, about 2,000 mm³ to about 2,200 mm³, about 2,000 mm³ to about 2,400 mm³, about 2,000 mm³ to about 2,600 mm³, about 2,000 mm³ to about 2,800 mm³, about 2,000 mm³ to about 3,000 mm³, about 2,000 mm³ to about 4,000 mm³, about 2,000 mm³ to about 5,000 mm³, about 2,000 mm³ to about 6,000 mm³, about 2,000 mm³ to about 7,000 mm³, about 2,000 mm³ to about 8,000 mm³, about 2,000 mm³ to about 9,000 mm³, about 2,000 mm³ to about 10,000 mm³, about 2,000 mm³ to about 12,000 mm³, about 2,000 mm³ to about 14,000 mm³, about 2,000 mm³ to about 16,000 mm³, about 2,000 mm³ to about 18,000 mm³, about 2,000 mm³ to about 20,000 mm³, about 2,000 mm³ to about 25,000 mm³, about 2,000 mm³ to about 30,000 mm³, about 2,000 mm³ to about 35,000 mm³, about 2,000 mm³ to about 40,000 mm³, about 2,000 mm³ to about 45,000 mm³, about 2,000 mm³ to about 50,000 mm³, about 2,000 mm³ to about 60,000 mm³, about 2,000 mm³ to about 70,000 mm³, about 2,000 mm³ to about 80,000 mm³, about 2,000 mm³ to about 90,000 mm³, about 2,200 mm³ to about 2,400 mm³, about 2,200 mm³ to about 2,600 mm³, about 2,200 mm³ to about 2,800 mm³, about 2,200 mm³ to about 3,000 mm³, about 2,200 mm³ to about 4,000 mm³, about 2,200 mm³ to about 5,000 mm³, about 2,200 mm³ to about 6,000 mm³, about 2,200 mm³ to about 7,000 mm³, about 2,200 mm³ to about 8,000 mm³, about 2,200 mm³ to about 9,000 mm³, about 2,200 mm³ to about 10,000 mm³, about 2,200 mm³ to about 12,000 mm³, about 2,200 mm³ to about 14,000 mm³, about 2,200 mm³ to about 16,000 mm³, about 2,200 mm³ to about 18,000 mm³, about 2,200 mm³ to about 20,000 mm³, about 2,200 mm³ to about 25,000 mm³, about 2,200 mm³ to about 30,000 mm³, about 2,200 mm³ to about 35,000 mm³, about 2,200 mm³ to about 40,000 mm³, about 2,200 mm³ to about 45,000 mm³, about 2,200 mm³ to about 50,000 mm³, about 2,200 mm³ to about 60,000 mm³, about 2,200 mm³ to about 70,000 mm³, about 2,200 mm³ to about 80,000 mm³, about 2,200 mm³ to about 90,000 mm³, about 2,400 mm³ to about 2,600 mm³, about 2,400 mm³ to about 2,800 mm³, about 2,400 mm³ to about 3,000 mm³, about 2,400 mm³ to about 4,000 mm³, about 2,400 mm³ to about 5,000 mm³, about 2,400 mm³ to about 6,000 mm³, about 2,400 mm³ to about 7,000 mm³, about 2,400 mm³ to about 8,000 mm³, about 2,400 mm³ to about 9,000 mm³, about 2,400 mm³ to about 10,000 mm³, about 2,400 mm³ to about 12,000 mm³, about 2,400 mm³ to about 14,000 mm³, about 2,400 mm³ to about 16,000 mm³, about 2,400 mm³ to about 18,000 mm³, about 2,400 mm³ to about 20,000 mm³, about 2,400 mm³ to about 25,000 mm³, about 2,400 mm³ to about 30,000 mm³, about 2,400 mm³ to about 35,000 mm³, about 2,400 mm³ to about 40,000 mm³, about 2,400 mm³ to about 45,000 mm³, about 2,400 mm³ to about 50,000 mm³, about 2,400 mm³ to about 60,000 mm³, about 2,400 mm³ to about 70,000 mm³, about 2,400 mm³ to about 80,000 mm³, about 2,400 mm³ to about 90,000 mm³, about 2,600 mm³ to about 2,800 mm³, about 2,600 mm³ to about 3,000 mm³, about 2,600 mm³ to about 4,000 mm³, about 2,600 mm³ to about 5,000 mm³, about 2,600 mm³ to about 6,000 mm³, about 2,600 mm³ to about 7,000 mm³, about 2,600 mm³ to about 8,000 mm³, about 2,600 mm³ to about 9,000 mm³, about 2,600 mm³ to about 10,000 mm³, about 2,600 mm³ to about 12,000 mm³, about 2,600 mm³ to about 14,000 mm³, about 2,600 mm³ to about 16,000 mm³, about 2,600 mm³ to about 18,000 mm³, about 2,600 mm³ to about 20,000 mm³, about 2,600 mm³ to about 25,000 mm³, about 2,600 mm³ to about 30,000 mm³, about 2,600 mm³ to about 35,000 mm³, about 2,600 mm³ to about 40,000 mm³, about 2,600 mm³ to about 45,000 mm³, about 2,600 mm³ to about 50,000 mm³, about 2,600 mm³ to about 60,000 mm³, about 2,600 mm³ to about 70,000 mm³, about 2,600 mm³ to about 80,000 mm³, about 2,600 mm³ to about 90,000 mm³, about 2,800 mm³ to about 3,000 mm³, about 2,800 mm³ to about 4,000 mm³, about 2,800 mm³ to about 5,000 mm³, about 2,800 mm³ to about 6,000 mm³, about 2,800 mm³ to about 7,000 mm³, about 2,800 mm³ to about 8,000 mm³, about 2,800 mm³ to about 9,000 mm³, about 2,800 mm³ to about 10,000 mm³, about 2,800 mm³ to about 12,000 mm³, about 2,800 mm³ to about 14,000 mm³, about 2,800 mm³ to about 16,000 mm³, about 2,800 mm³ to about 18,000 mm³, about 2,800 mm³ to about 20,000 mm³, about 2,800 mm³ to about 25,000 mm³, about 2,800 mm³ to about 30,000 mm³, about 2,800 mm³ to about 35,000 mm³, about 2,800 mm³ to about 40,000 mm³, about 2,800 mm³ to about 45,000 mm³, about 2,800 mm³ to about 50,000 mm³, about 2,800 mm³ to about 60,000 mm³, about 2,800 mm³ to about 70,000 mm³, about 2,800 mm³ to about 80,000 mm³, about 2,800 mm³ to about 90,000 mm³, about 3,000 mm³ to about 4,000 mm³, about 3,000 mm³ to about 5,000 mm³, about 3,000 mm³ to about 6,000 mm³, about 3,000 mm³ to about 7,000 mm³, about 3,000 mm³ to about 8,000 mm³, about 3,000 mm³ to about 9,000 mm³, about 3,000 mm³ to about 10,000 mm³, about 3,000 mm³ to about 12,000 mm³, about 3,000 mm³ to about 14,000 mm³, about 3,000 mm³ to about 16,000 mm³, about 3,000 mm³ to about 18,000 mm³, about 3,000 mm³ to about 20,000 mm³, about 3,000 mm³ to about 25,000 mm³, about 3,000 mm³ to about 30,000 mm³, about 3,000 mm³ to about 35,000 mm³, about 3,000 mm³ to about 40,000 mm³, about 3,000 mm³ to about 45,000 mm³, about 3,000 mm³ to about 50,000 mm³, about 3,000 mm³ to about 60,000 mm³, about 3,000 mm³ to about 70,000 mm³, about 3,000 mm³ to about 80,000 mm³, about 3,000 mm³ to about 90,000 mm³, about 4,000 mm³ to about 5,000 mm³, about 4,000 mm³ to about 6,000 mm³, about 4,000 mm³ to about 7,000 mm³, about 4,000 mm³ to about 8,000 mm³, about 4,000 mm³ to about 9,000 mm³, about 4,000 mm³ to about 10,000 mm³, about 4,000 mm³ to about 12,000 mm³, about 4,000 mm³ to about 14,000 mm³, about 4,000 mm³ to about 16,000 mm³, about 4,000 mm³ to about 18,000 mm³, about 4,000 mm³ to about 20,000 mm³, about 4,000 mm³ to about 25,000 mm³, about 4,000 mm³ to about 30,000 mm³, about 4,000 mm³ to about 35,000 mm³, about 4,000 mm³ to about 40,000 mm³, about 4,000 mm³ to about 45,000 mm³, about 4,000 mm³ to about 50,000 mm³, about 4,000 mm³ to about 60,000 mm³, about 4,000 mm³ to about 70,000 mm³, about 4,000 mm³ to about 80,000 mm³, about 4,000 mm³ to about 90,000 mm³, about 5,000 mm³ to about 6,000 mm³, about 5,000 mm³ to about 7,000 mm³, about 5,000 mm³ to about 8,000 mm³, about 5,000 mm³ to about 9,000 mm³, about 5,000 mm³ to about 10,000 mm³, about 5,000 mm³ to about 12,000 mm³, about 5,000 mm³ to about 14,000 mm³, about 5,000 mm³ to about 16,000 mm³, about 5,000 mm³ to about 18,000 mm³, about 5,000 mm³ to about 20,000 mm³, about 5,000 mm³ to about 25,000 mm³, about 5,000 mm³ to about 30,000 mm³, about 5,000 mm³ to about 35,000 mm³, about 5,000 mm³ to about 40,000 mm³, about 5,000 mm³ to about 45,000 mm³, about 5,000 mm³ to about 50,000 mm³, about 5,000 mm³ to about 60,000 mm³, about 5,000 mm³ to about 70,000 mm³, about 5,000 mm³ to about 80,000 mm³, about 5,000 mm³ to about 90,000 mm³, about 6,000 mm³ to about 7,000 mm³, about 6,000 mm³ to about 8,000 mm³, about 6,000 mm³ to about 9,000 mm³, about 6,000 mm³ to about 10,000 mm³, about 6,000 mm³ to about 12,000 mm³, about 6,000 mm³ to about 14,000 mm³, about 6,000 mm³ to about 16,000 mm³, about 6,000 mm³ to about 18,000 mm³, about 6,000 mm³ to about 20,000 mm³, about 6,000 mm³ to about 25,000 mm³, about 6,000 mm³ to about 30,000 mm³, about 6,000 mm³ to about 35,000 mm³, about 6,000 mm³ to about 40,000 mm³, about 6,000 mm³ to about 45,000 mm³, about 6,000 mm³ to about 50,000 mm³, about 6,000 mm³ to about 60,000 mm³, about 6,000 mm³ to about 70,000 mm³, about 6,000 mm³ to about 80,000 mm³, about 6,000 mm³ to about 90,000 mm³, about 7,000 mm³ to about 8,000 mm³, about 7,000 mm³ to about 9,000 mm³, about 7,000 mm³ to about 10,000 mm³, about 7,000 mm³ to about 12,000 mm³, about 7,000 mm³ to about 14,000 mm³, about 7,000 mm³ to about 16,000 mm³, about 7,000 mm³ to about 18,000 mm³, about 7,000 mm³ to about 20,000 mm³, about 7,000 mm³ to about 25,000 mm³, about 7,000 mm³ to about 30,000 mm³, about 7,000 mm³ to about 35,000 mm³, about 7,000 mm³ to about 40,000 mm³, about 7,000 mm³ to about 45,000 mm³, about 7,000 mm³ to about 50,000 mm³, about 7,000 mm³ to about 60,000 mm³, about 7,000 mm³ to about 70,000 mm³, about 7,000 mm³ to about 80,000 mm³, about 7,000 mm³ to about 90,000 mm³, about 8,000 mm³ to about 9,000 mm³, about 8,000 mm³ to about 10,000 mm³, about 8,000 mm³ to about 12,000 mm³, about 8,000 mm³ to about 14,000 mm³, about 8,000 mm³ to about 16,000 mm³, about 8,000 mm³ to about 18,000 mm³, about 8,000 mm³ to about 20,000 mm³, about 8,000 mm³ to about 25,000 mm³, about 8,000 mm³ to about 30,000 mm³, about 8,000 mm³ to about 35,000 mm³, about 8,000 mm³ to about 40,000 mm³, about 8,000 mm³ to about 45,000 mm³, about 8,000 mm³ to about 50,000 mm³, about 8,000 mm³ to about 60,000 mm³, about 8,000 mm³ to about 70,000 mm³, about 8,000 mm³ to about 80,000 mm³, about 8,000 mm³ to about 90,000 mm³, about 9,000 mm³ to about 10,000 mm³, about 9,000 mm³ to about 12,000 mm³, about 9,000 mm³ to about 14,000 mm³, about 9,000 mm³ to about 16,000 mm³, about 9,000 mm³ to about 18,000 mm³, about 9,000 mm³ to about 20,000 mm³, about 9,000 mm³ to about 25,000 mm³, about 9,000 mm³ to about 30,000 mm³, about 9,000 mm³ to about 35,000 mm³, about 9,000 mm³ to about 40,000 mm³, about 9,000 mm³ to about 45,000 mm³, about 9,000 mm³ to about 50,000 mm³, about 9,000 mm³ to about 60,000 mm³, about 9,000 mm³ to about 70,000 mm³, about 9,000 mm³ to about 80,000 mm³, about 9,000 mm³ to about 90,000 mm³, about 10,000 mm³ to about 12,000 mm³, about 10,000 mm³ to about 14,000 mm³, about 10,000 mm³ to about 16,000 mm³, about 10,000 mm³ to about 18,000 mm³, about 10,000 mm³ to about 20,000 mm³, about 10,000 mm³ to about 25,000 mm³, about 10,000 mm³ to about 30,000 mm³, about 10,000 mm³ to about 35,000 mm³, about 10,000 mm³ to about 40,000 mm³, about 10,000 mm³ to about 45,000 mm³, about 10,000 mm³ to about 50,000 mm³, about 10,000 mm³ to about 60,000 mm³, about 10,000 mm³ to about 70,000 mm³, about 10,000 mm³ to about 80,000 mm³, about 10,000 mm³ to about 90,000 mm³, about 12,000 mm³ to about 14,000 mm³, about 12,000 mm³ to about 16,000 mm³, about 12,000 mm³ to about 18,000 mm³, about 12,000 mm³ to about 20,000 mm³, about 12,000 mm³ to about 25,000 mm³, about 12,000 mm³ to about 30,000 mm³, about 12,000 mm³ to about 35,000 mm³, about 12,000 mm³ to about 40,000 mm³, about 12,000 mm³ to about 45,000 mm³, about 12,000 mm³ to about 50,000 mm³, about 12,000 mm³ to about 60,000 mm³, about 12,000 mm³ to about 70,000 mm³, about 12,000 mm³ to about 80,000 mm³, about 12,000 mm³ to about 90,000 mm³, about 14,000 mm³ to about 16,000 mm³, about 14,000 mm³ to about 18,000 mm³, about 14,000 mm³ to about 20,000 mm³, about 14,000 mm³ to about 25,000 mm³, about 14,000 mm³ to about 30,000 mm³, about 14,000 mm³ to about 35,000 mm³, about 14,000 mm³ to about 40,000 mm³, about 14,000 mm³ to about 45,000 mm³, about 14,000 mm³ to about 50,000 mm³, about 14,000 mm³ to about 60,000 mm³, about 14,000 mm³ to about 70,000 mm³, about 14,000 mm³ to about 80,000 mm³, about 14,000 mm³ to about 90,000 mm³, about 16,000 mm³ to about 18,000 mm³, about 16,000 mm³ to about 20,000 mm³, about 16,000 mm³ to about 25,000 mm³, about 16,000 mm³ to about 30,000 mm³, about 16,000 mm³ to about 35,000 mm³, about 16,000 mm³ to about 40,000 mm³, about 16,000 mm³ to about 45,000 mm³, about 16,000 mm³ to about 50,000 mm³, about 16,000 mm³ to about 60,000 mm³, about 16,000 mm³ to about 70,000 mm³, about 16,000 mm³ to about 80,000 mm³, about 16,000 mm³ to about 90,000 mm³, about 18,000 mm³ to about 20,000 mm³, about 18,000 mm³ to about 25,000 mm³, about 18,000 mm³ to about 30,000 mm³, about 18,000 mm³ to about 35,000 mm³, about 18,000 mm³ to about 40,000 mm³, about 18,000 mm³ to about 45,000 mm³, about 18,000 mm³ to about 50,000 mm³, about 18,000 mm³ to about 60,000 mm³, about 18,000 mm³ to about 70,000 mm³, about 18,000 mm³ to about 80,000 mm³, about 18,000 mm³ to about 90,000 mm³, about 20,000 mm³ to about 25,000 mm³, about 20,000 mm³ to about 30,000 mm³, about 20,000 mm³ to about 35,000 mm³, about 20,000 mm³ to about 40,000 mm³, about 20,000 mm³ to about 45,000 mm³, about 20,000 mm³ to about 50,000 mm³, about 20,000 mm³ to about 60,000 mm³, about 20,000 mm³ to about 70,000 mm³, about 20,000 mm³ to about 80,000 mm³, about 20,000 mm³ to about 90,000 mm³, about 25,000 mm³ to about 30,000 mm³, about 25,000 mm³ to about 35,000 mm³, about 25,000 mm³ to about 40,000 mm³, about 25,000 mm³ to about 45,000 mm³, about 25,000 mm³ to about 50,000 mm³, about 25,000 mm³ to about 60,000 mm³, about 25,000 mm³ to about 70,000 mm³, about 25,000 mm³ to about 80,000 mm³, about 25,000 mm³ to about 90,000 mm³, about 30,000 mm³ to about 35,000 mm³, about 30,000 mm³ to about 40,000 mm³, about 30,000 mm³ to about 45,000 mm³, about 30,000 mm³ to about 50,000 mm³, about 30,000 mm³ to about 60,000 mm³, about 30,000 mm³ to about 70,000 mm³, about 30,000 mm³ to about 80,000 mm³, about 30,000 mm³ to about 90,000 mm³, about 35,000 mm³ to about 40,000 mm³, about 35,000 mm³ to about 45,000 mm³, about 35,000 mm³ to about 50,000 mm³, about 35,000 mm³ to about 60,000 mm³, about 35,000 mm³ to about 70,000 mm³, about 35,000 mm³ to about 80,000 mm³, about 35,000 mm³ to about 90,000 mm³, about 40,000 mm³ to about 45,000 mm³, about 40,000 mm³ to about 50,000 mm³, about 40,000 mm³ to about 60,000 mm³, about 40,000 mm³ to about 70,000 mm³, about 40,000 mm³ to about 80,000 mm³, about 40,000 mm³ to about 90,000 mm³, about 45,000 mm³ to about 50,000 mm³, about 45,000 mm³ to about 60,000 mm³, about 45,000 mm³ to about 70,000 mm³, about 45,000 mm³ to about 80,000 mm³, about 45,000 mm³ to about 90,000 mm³, about 50,000 mm³ to about 60,000 mm³, about 50,000 mm³ to about 70,000 mm³, about 50,000 mm³ to about 80,000 mm³, about 50,000 mm³ to about 90,000 mm³, about 60,000 mm³ to about 70,000 mm³, about 60,000 mm³ to about 80,000 mm³, about 60,000 mm³ to about 90,000 mm³, about 70,000 mm³ to about 80,000 mm³, about 70,000 mm³ to about 90,000 mm³, or about 80,000 mm³ to about 90,000 mm³.

In other aspects of this embodiment, in an endoscope device 120 disclosed herein the suction channel 190 is annular with an inside diameter between 1 mm and 10 mm. As such, the suction channel 190 has a diameter of, e.g., about 1 mm, about 2 mm, about 3 mm, about 4 mm, about 5 mm, about 6 mm, about 7 mm, about 8 mm, about 9 mm, or about 10 mm. In further aspects of this embodiment, in an endoscope device 120 disclosed herein the suction channel 190 has a diameter of, e.g., at least 1 mm, at least 2 mm, at least 3 mm, at least 4 mm, at least 5 mm, at least 6 mm, at least 7 mm, at least 8 mm, at least 9 mm, or at least 10 mm. In still further aspects of this embodiment, in an endoscope device 120 disclosed herein the suction channel 190 has a diameter of, e.g., at most 1 mm, at most 2 mm, at most 3 mm, at most 4 mm, at most 5 mm, at most 6 mm, at most 7 mm, at most 8 mm, at most 9 mm, or at most 10 mm.

In further aspects of this embodiment, in an endoscope device 120 disclosed herein the suction channel 190 has a diameter of, e.g., about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm, or about 9 mm to about 10 mm.

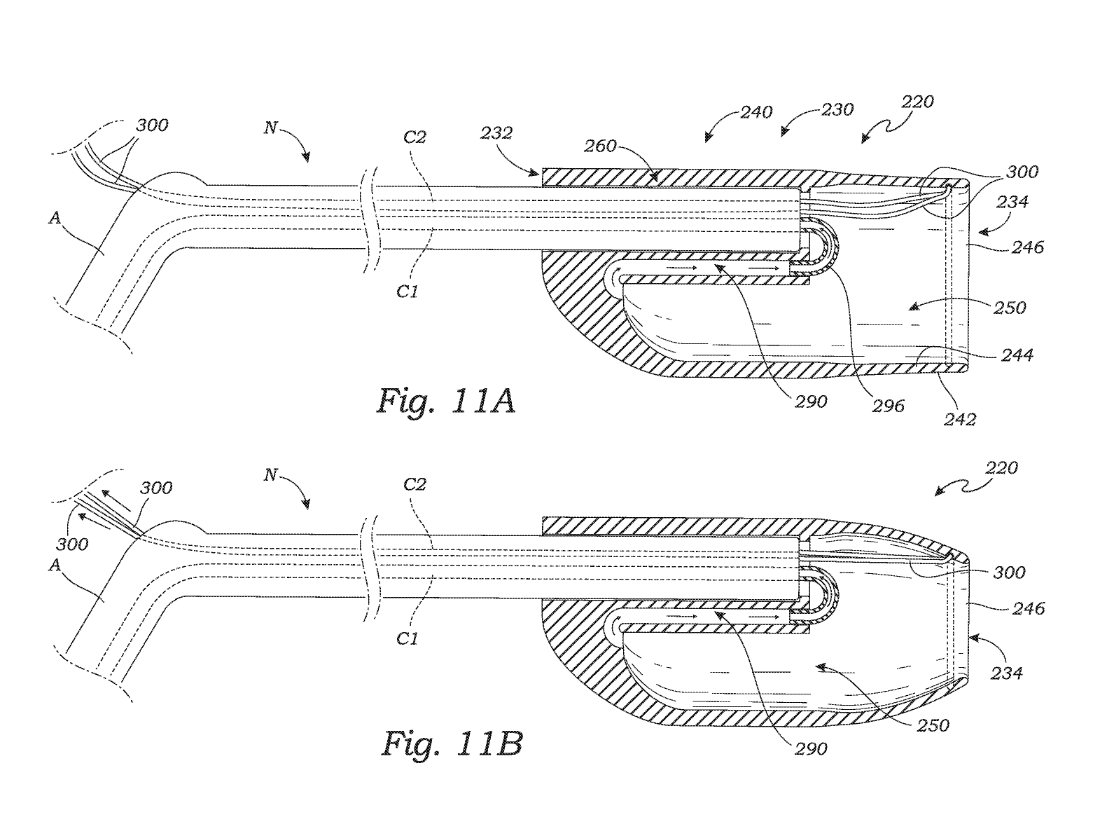

Referring now to FIGS. 11A and 11B there are illustrated side cross-sectional views of a further alternative exemplary endoscope device 220 with an endoscope N inserted therein and in first and second modes of operation, respectively, in accordance with at least one embodiment. As will be seen and appreciated, the configuration of the device 220 is analogous to that of FIG. 8, particularly at the proximal end thereof and in connection with the configurations of the primary and secondary lumens and the incorporation of a suction channel 290 and suction tube 296 therebetween. The primary lumen 250 again has a proximal portion that is substantially adjacent to the secondary lumen 260 and the endoscope N and a distal portion essentially in front of or distal of the endoscope N. Accordingly, for illustration, the device 220 may dimensionally be similar to that of FIG. 8 as set forth above, though those skilled in the art will appreciate that as with all other embodiments herein, a variety of other configurations are possible, such that those illustrated are to be understood as exemplary and non-limiting. Notably, and how the alternative embodiment of FIGS. 11A and 11B primarily differs from the others, including that of FIGS. 8-10C, is in that not only is the wall 240 of the body 230 tapered, but the actual wall thickness, defined at any point along the wall 240 as the distance thereacross between the outer and inner surfaces 242, 244, is reduced toward the distal opening 234 and nose 246, resulting in a relatively thin and more flexible distal end of the device 220. Moreover, while such a construction alone may enable insertion of even a relatively large device 220 in relatively tight anatomy, there is further provided in conjunction with the thin-wall distal body 230 a filament 300 coupled to the nose 246 in a drawstring fashion with the filament 300 stitched about or threaded through a casing about the thin-wall distal body 230. Applying tension to the filament 300 reduces the size of the distal opening 234 and effectively that of the primary lumen 250, the filament 300 being configured to operably pass through a channel C2 of the endoscope N selectively inserted in the secondary lumen 260 of the device 220. In FIG. 11A, the device 220 is shown in its “at rest” or natural state wherein no tension is applied to the filament 300 and so the wall 240 being biased outwardly effectively maintains the distal opening 234 in its “unactivated” relatively larger configuration, preferably at a maximum or near maximum opening size. This would be the operational mode of the device 220 once positioned within the anatomy and ready for suction and retrieval, the distal end being “opened” for receipt of material. In FIG. 11B, a view similar to that of FIG. 11A is shown, only now with tension applied to the filaments 300 back at the proximal end of the scope N, at the handle A, which tension or force is transmitted along the filaments 300 that are passed through a second channel C2 of the scope N so as to pull the distal wall 240 radially inward and thereby reduce or move in the direction of closing off the distal opening 234 to the primary lumen 250, which in turn also serves to further taper or curve the outside surface 242 of the body wall 240 and render the overall profile of the device 220 relatively more tapered or “bullet nosed.” It will be appreciated that such a second mode of operation of the endoscope device 220 positions the device for insertion as being more tapered or streamlined and also may be the “activated” shape or configuration of the device 220 for removal, with the relatively smaller distal opening 234 cooperating with the continued application of suction, as indicated by the arrows in the suction channel 290 in FIG. 11B, to retain whatever material has been retrieved as the device 220 is extubated or removed. It will be further appreciated that while a “purse string” or drawstring style selective closure is thus incorporated within the embodiment of the endoscope device 220 shown and described in connection with FIGS. 11A and 11B, other such biasing and selective closure means, now known or later developed, may be employed as well. For example, a mesh spring type configuration of the distal body wall would result in a stent-like functionality that would be in an expanded “at rest” configuration and would be pulled into a relatively compressed or reduced size or configuration when the device 220 is to be inserted or removed.

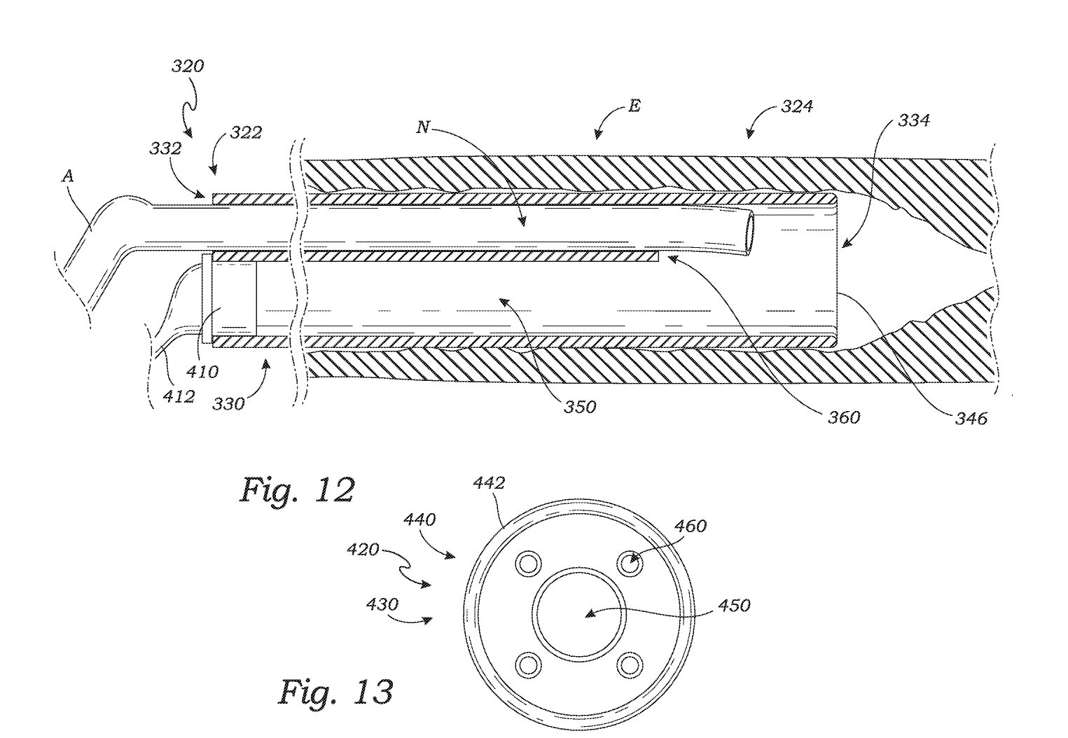

Turning next to FIG. 12, there is shown a partial cross-sectional view of a still further alternative exemplary embodiment of an endoscope device 320 with an endoscope N inserted therein. Here, rather than the device being an attachment fitted on the distal or working end of the endoscope N, the device 320 is effectively an overtube body 330 with a secondary lumen 360 or channel within which the scope N slidably operates during use. The primary lumen 350 again defines the effective suction path through the device 320, which here runs the length of the tubular body 330 as shown. Accordingly, with both the endoscope N itself via its handle A and the proximal end 322 of the device 320 being accessible outside the anatomy throughout use, it will be appreciated that with the position of the distal end 324 of the device 320, and the nose 346 particularly, being subject to independent control, or control or manipulation separate from the scope N itself, the scope N may here have a relatively looser clearance within the secondary lumen 360, rather than a frictional fit that would thereby limit the movement of the scope N relative to the device 320. Once again as shown, the secondary lumen 360 may be offset from the primary lumen 350, whereby visualization and other work via the endoscope N is unaffected even as suction is applied and potentially significant amounts of impaction are retrieved. In fact, in the exemplary embodiment, it is contemplated that a separate suction line 412 be connected in fluid communication with the primary lumen 350 through a suction attachment 410 removably and sealingly engageable with the body 330 so as to apply direct suction to the primary lumen 350, which suction is then felt or operative at the opposite distal opening 334 of the device 320, again all under visualization from the scope N positioned within the secondary lumen 360, which lumen serves as a separate and substantially “clean” track for the scope N even as the primary lumen 350 is occupied with suctioning. Accordingly, any suction port of the endoscope N is not even necessarily utilized. It follows from the foregoing that the endoscope N may be further manipulated and advanced to illuminate and perform other operations within the anatomy independent of the suction event and the retrieval of any food impaction or the like in and through the device 320, and the primary lumen 350 specifically. In such alternative embodiment, rather than the primary lumen being completely or even partially distal of the secondary lumen, here the primary and secondary lumens 350, 360 are substantially coterminous at least proximally. As shown in FIG. 12, in the exemplary embodiment, the two lumens 350, 360 are not coterminous distally, with the secondary lumen 360 offset from and short of the distal opening 334 and nose 346 with which the primary lumen 350 is contiguous so as to again provide a volumetric work space within the primary lumen 350 of the device 320, into which space it may be desirable to selectively advance the scope N. While a particular configuration and proportionality of the primary and secondary lumens 350, 360 of the exemplary endoscope device 320 are shown, it will be appreciated that such is merely illustrative and non-limiting.

By way of illustration, the width or diameter of both the primary lumen 350 and the secondary lumen 360 may substantially correspond to the dimensions noted for the primary and secondary lumens 50, 60 of the embodiment of the device 20 shown in FIGS. 1-7C, which dimensional ranges are not repeated here. As for the length of the primary lumen 350 and hence the overall device 320, it will be appreciated that this is primarily dictated by anatomical considerations as they relate to the intended uses of the device 320, but for these purposes the overall length of the device 320 and thus of the primary lumen 350 is in the range of 400 mm to 800 mm, while the length of the secondary lumen 360 may be 350 mm to 800 mm, effectively indicating up to a 50 mm set back of the end of the secondary lumen 360 and thus potentially the scope N, at least related to its substantially free movement outside of the secondary lumen 360. However, even these ranges can be increased depending on anatomical variations and the type of endoscope N employed, among other factors.

In related aspects of this embodiment, in an endoscope device 320 disclosed herein the length of the body 330 and thus the primary lumen 350 is, e.g., about 400 mm, about 450 mm, about 500 mm, about 550 mm, about 600 mm, about 650 mm, about 700 mm, about 750 mm, or about 800 mm. In further aspects of this embodiment, in an endoscope device 320 disclosed herein the length of the body 330 and thus the primary lumen 350 is, e.g., at least 400 mm, at least 450 mm, at least 500 mm, at least 550 mm, at least 600 mm, at least 650 mm, at least 700 mm, at least 750 mm, or at least 800 mm. In still further aspects of this embodiment, in an endoscope device 320 disclosed herein the length of the body 330 and thus the primary lumen 350 is, e.g., at most 400 mm, at most 450 mm, at most 500 mm, at most 550 mm, at most 600 mm, at most 650 mm, at most 700 mm, at most 750 mm, or at most 800 mm.

In further related aspects of this embodiment, in an endoscope device 320 disclosed herein the length of the body 330 and thus the primary lumen 350 is, e.g., about 400 mm to about 450 mm, about 400 mm to about 500 mm, about 400 mm to about 550 mm, about 400 mm to about 600 mm, about 400 mm to about 650 mm, about 400 mm to about 700 mm, about 400 mm to about 750 mm, about 400 mm to about 800 mm, about 450 mm to about 500 mm, about 450 mm to about 550 mm, about 450 mm to about 600 mm, about 450 mm to about 650 mm, about 450 mm to about 700 mm, about 450 mm to about 750 mm, about 450 mm to about 800 mm, about 500 mm to about 550 mm, about 500 mm to about 600 mm, about 500 mm to about 650 mm, about 500 mm to about 700 mm, about 500 mm to about 750 mm, about 500 mm to about 800 mm, about 550 mm to about 600 mm, about 550 mm to about 650 mm, about 550 mm to about 700 mm, about 550 mm to about 750 mm, about 550 mm to about 800 mm, about 600 mm to about 650 mm, about 600 mm to about 700 mm, about 600 mm to about 750 mm, about 600 mm to about 800 mm, about 650 mm to about 700 mm, about 650 mm to about 750 mm, about 650 mm to about 800 mm, about 700 mm to about 750 mm, about 700 mm to about 800 mm, or about 750 mm to about 800 mm.

In still further related aspects of this embodiment, in an endoscope device 320 disclosed herein the offset from the secondary lumen 360 to the nose 346 is, e.g., about 0 mm, about 5 mm, about 10 mm, about 15 mm, about 20 mm, about 25 mm, about 30 mm, about 35 mm, about 40 mm, about 45 mm, or about 50 mm. In further aspects of this embodiment, in an endoscope device 320 disclosed herein the offset from the secondary lumen 360 to the nose 346 is, e.g., at least 0 mm, at least 5 mm, at least 10 mm, at least 15 mm, at least 20 mm, at least 25 mm, at least 30 mm, at least 35 mm, at least 40 mm, at least 45 mm, or at least 50 mm. In further aspects of this embodiment, in an endoscope device 320 disclosed herein the offset from the secondary lumen 360 to the nose 346 is, e.g., at most 0 mm, at most 5 mm, at most 10 mm, at most 15 mm, at most 20 mm, at most 25 mm, at most 30 mm, at most 35 mm, at most 40 mm, at most 45 mm, or at most 50 mm.

In further related aspects of this embodiment, in an endoscope device 320 disclosed herein the offset from the secondary lumen 360 to the nose 346 is, e.g., about 0 mm to about 5 mm, about 0 mm to about 10 mm, about 0 mm to about 15 mm, about 0 mm to about 20 mm, about 0 mm to about 25 mm, about 0 mm to about 30 mm, about 0 mm to about 35 mm, about 0 mm to about 40 mm, about 0 mm to about 45 mm, about 0 mm to about 50 mm, about 5 mm to about 10 mm, about 5 mm to about 15 mm, about 5 mm to about 20 mm, about 5 mm to about 25 mm, about 5 mm to about 30 mm, about 5 mm to about 35 mm, about 5 mm to about 40 mm, about 5 mm to about 45 mm, about 5 mm to about 50 mm, about 10 mm to about 15 mm, about 10 mm to about 20 mm, about 10 mm to about 25 mm, about 10 mm to about 30 mm, about 10 mm to about 35 mm, about 10 mm to about 40 mm, about 10 mm to about 45 mm, about 10 mm to about 50 mm, about 15 mm to about 20 mm, about 15 mm to about 25 mm, about 15 mm to about 30 mm, about 15 mm to about 35 mm, about 15 mm to about 40 mm, about 15 mm to about 45 mm, about 15 mm to about 50 mm, about 20 mm to about 25 mm, about 20 mm to about 30 mm, about 20 mm to about 35 mm, about 20 mm to about 40 mm, about 20 mm to about 45 mm, about 20 mm to about 50 mm, about 25 mm to about 30 mm, about 25 mm to about 35 mm, about 25 mm to about 40 mm, about 25 mm to about 45 mm, about 25 mm to about 50 mm, about 30 mm to about 35 mm, about 30 mm to about 40 mm, about 30 mm to about 45 mm, about 30 mm to about 50 mm, about 35 mm to about 40 mm, about 35 mm to about 45 mm, about 35 mm to about 50 mm, about 40 mm to about 45 mm, about 40 mm to about 50 mm, or about 45 mm to about 50 mm.

Finally, referring to FIG. 13, there is shown an end view of a further exemplary alternative embodiment of an endoscope device 420 according to aspects of the present invention, here in the form of a complete “purpose-built” scope. Most notably, the device 420 comprises a primary lumen 450 for suction that is the largest of all the channels of the scope, i.e., the other secondary lumen or lumens 460 also configured within the device 420. While the primary lumen 450 is depicted as being annular and located centrally within the endoscope device 420, it will be appreciated that the invention is not so limited. Rather, the primary lumen 450 can take on a number of shapes and locations within the device 420 without departing from the spirit and scope of the invention. As shown, the primary lumen 450 is centrally located and of a diameter roughly half of the entire device 420 as formed by an outer body 430 having an outer wall 440 that serves to “bundle” all lumens 450, 460 together. Again, any such sizes and arrangements of individual lumens and the resulting overall device 420 are merely illustrative and non-limiting. One aspect of the invention, however, is trading flexibility for increased suction lumen size to better suit applications such as described herein where a foreign object or food impaction is to be removed from a patient's esophagus. Accordingly, the overall outside diameter may be on the order of 50 mm and an overall length again ranging from 400 mm to 800 mm as with the embodiment of FIG. 12, on which basis such dimensional ranges for the length will not be repeated here. It will be appreciated that in the typical “upper GI” intubation, as in inserting a device into the esophagus and perhaps the stomach but no further, significant manipulation of the scope is not usually necessary. So, here, in the exemplary alternative endoscope device 420 manipulability and length have been traded for a significantly larger suction port or primary lumen 450 while still offering sufficient length and mobility for the anticipated uses.

In other aspects of this embodiment, in an endoscope device 420 disclosed herein the primary lumen 450 is annular with an inside diameter between 4 mm and 30 mm. As such, the primary lumen 450 has a diameter of, e.g., about 4 mm, about 6 mm, about 8 mm, about 10 mm, about 12 mm, about 14 mm, about 16 mm, about 18 mm, about 20 mm, about 22 mm, about 24 mm, about 26 mm, about 28 mm, or about 30 mm. In further aspects of this embodiment, in an endoscope device 420 disclosed herein the primary lumen 450 has a diameter of, e.g., at least 4 mm, at least 6 mm, at least 8 mm, at least 10 mm, at least 12 mm, at least 14 mm, at least 16 mm, at least 18 mm, at least 20 mm, at least 22 mm, at least 24 mm, at least 26 mm, at least 28 mm, or at least 30 mm. In still further aspects of this embodiment, in an endoscope device 420 disclosed herein the primary lumen 450 has a diameter of, e.g., at most 4 mm, at most 6 mm, at most 8 mm, at most 10 mm, at most 12 mm, at most 14 mm, at most 16 mm, at most 18 mm, at most 20 mm, at most 22 mm, at most 24 mm, at most 26 mm, at most 28 mm, or at most 30 mm.

In further aspects of this embodiment, in an endoscope device 420 disclosed herein the primary lumen 450 has a diameter of, e.g., about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 4 mm to about 22 mm, about 4 mm to about 24 mm, about 4 mm to about 26 mm, about 4 mm to about 28 mm, about 4 mm to about 30 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 6 mm to about 22 mm, about 6 mm to about 24 mm, about 6 mm to about 26 mm, about 6 mm to about 28 mm, about 6 mm to about 30 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 8 mm to about 22 mm, about 8 mm to about 24 mm, about 8 mm to about 26 mm, about 8 mm to about 28 mm, about 8 mm to about 30 mm, about 10 mm to about 12 mm, about 10 mm to about 14 mm, about 10 mm to about 16 mm, about 10 mm to about 18 mm, about 10 mm to about 20 mm, about 10 mm to about 22 mm, about 10 mm to about 24 mm, about 10 mm to about 26 mm, about 10 mm to about 28 mm, about 10 mm to about 30 mm, about 12 mm to about 14 mm, about 12 mm to about 16 mm, about 12 mm to about 18 mm, about 12 mm to about 20 mm, about 12 mm to about 22 mm, about 12 mm to about 24 mm, about 12 mm to about 26 mm, about 12 mm to about 28 mm, about 12 mm to about 30 mm, about 14 mm to about 16 mm, about 14 mm to about 18 mm, about 14 mm to about 20 mm, about 14 mm to about 22 mm, about 14 mm to about 24 mm, about 14 mm to about 26 mm, about 14 mm to about 28 mm, about 14 mm to about 30 mm, about 16 mm to about 18 mm, about 16 mm to about 20 mm, about 16 mm to about 22 mm, about 16 mm to about 24 mm, about 16 mm to about 26 mm, about 16 mm to about 28 mm, about 16 mm to about 30 mm, about 18 mm to about 20 mm, about 18 mm to about 22 mm, about 18 mm to about 24 mm, about 18 mm to about 26 mm, about 18 mm to about 28 mm, about 18 mm to about 30 mm, about 20 mm to about 22 mm, about 20 mm to about 24 mm, about 20 mm to about 26 mm, about 20 mm to about 28 mm, about 20 mm to about 30 mm, about 22 mm to about 24 mm, about 22 mm to about 26 mm, about 22 mm to about 28 mm, about 22 mm to about 30 mm, about 24 mm to about 26 mm, about 24 mm to about 28 mm, about 24 mm to about 30 mm, about 26 mm to about 28 mm, about 26 mm to about 30 mm, or about 28 mm to about 30 mm.

It will again be appreciated that a variety of other arrangements for the various lumens 450, 460 within the endoscope device 420 are possible, such that the disclosed arrangement and size range is to be understood as merely illustrative and non-limiting. Moreover, any such “purpose-built scope” device 420 can include various other lumens or channels to suit a particular purpose, including visualization, illumination, irrigation, and passage of a tool, hence the illustrated four secondary lumens 460 surrounding the main suction lumen 450. For visualization, rather than fiberoptics, the endoscope device 420 may be equipped with any digital imaging technology now know or later developed, including but not limited to CCD.

In any of the foregoing embodiments or other related embodiments, it will be appreciated that any other tools or techniques now known or later developed for addressing particularly a food impaction, such as means by which the impaction is sectioned into smaller portions, softened, etc., may be employed in combination with an endoscope device according to aspects of the present invention.

### EXAMPLES

The following non-limiting examples are provided for illustrative purposes only in order to facilitate a more complete understanding of the disclosed subject matter. These examples should not be construed to limit any of the embodiments described in the present specification, including those pertaining to an endoscope device, kits comprising an endoscope device, and/or methods and uses of such an endoscope device.

### Example 1

### Meat Bolus

This example demonstrates the use of an endoscope device according to aspects of the present invention in the non-surgical removal of meat bolus from a patient's esophagus.

A 52-year-old male presented with diffuse chest pain or pressure, throat pain and difficulty swallowing, and a sensation of choking. Upon a complete medical work-up and examination, including a health history indicating no prior instances of trouble with ingestion and review with the patient indicating a recent meal including steak and that the symptoms began soon thereafter, the clinician concluded likely food impaction within the esophagus in the form of meat (steak) bolus and prepped the patient for endoscopy. Accordingly, the clinician fitted a standard adult gastroscope having a nominal 10 mm outside diameter with an appropriately sized distal endoscope device attachment stepping up to a distal lumen having an opening of 25 mm and length of 50 mm and a nominal outside diameter of 28 mm and length of 75 mm, yielding a volume of approximately 25,000 mm³, which equates to roughly one-and-a-half cubic inches (1% in³) or two tablespoons of material, something the clinician felt was likely sufficient to retrieve the meat bolus in one pass. With the patient under sedation, the clinician lubricated the gastroscope and retriever attachment and intubated the scope into the esophagus as by passing through the oral cavity and following the hard palate into and through the pharynx and then into the esophagus through the upper esophageal sphincter all under clear and complete visualization and then advanced the scope distally until the retriever attachment was positioned just above the meat bolus that appeared to be lodged on or in the lower esophageal sphincter. Then applying continuous vacuum of 150 mm Hg pressure to the retriever device through the suction port of the gastroscope, the clinician was able to pull the bolus into the distal lumen of the retriever attachment and with suction still applied extubate the scope and thereby remove the food bolus also as shielded by the retriever and so without any trauma to the esophagus or other anatomy. Post-procedure, the patient presented with no complications and indicated only minor throat soreness.

### Example 2

### Esophageal Stricture and Food Impaction

This example demonstrates the use of an endoscope device according to aspects of the present invention in the non-surgical removal of a food impaction caused by esophageal stricture.

A 61-year-old female presented with difficulty swallowing and reflux and regurgitation symptoms. Through a complete medical work-up and health history it was determined that the patient had for years been treated for cirrhosis of the liver with the scarring in the liver cutting down on blood flow in the liver and so leading to increased blood flow through the veins of the esophagus, resulting in esophageal varices. The clinician concluded pre-endoscopy that the treatment of the varices had likely led to esophageal stricture, or narrowing of the esophagus, which was likely the cause of the patient's present symptoms. The patient was prepped and sedated, and the clinician selected a standard adult endoscope having a nominal 10 mm outside diameter with an appropriately sized distal endoscope device attachment configured with an offset secondary lumen into which the distal end of the endoscope was inserted and an adjacent and distal primary lumen having a nominal diameter of 20 mm and a volume of 20,000 mm³, of which approximately half or 10,000 mm³ is associated with the proximal space within the primary lumen adjacent the secondary lumen. By installing the retriever attachment on the distal end of the endoscope as by a frictional engagement with a mechanical stop and then connecting a suction tube from the suction port of the scope to a suction channel of the retriever, the device is configured to apply suction at the proximal-most location within the primary lumen and thereby pull material into the device while maintaining visualization. Accordingly, with the endoscope prepped, the clinician proceed with the first intubation and successfully passed the scope with device into the esophagus and visually observed the food impaction at a location within the esophagus intermediate of the upper and lower esophageal sphincters. After fifteen passes with uncompromised visualization, the clinician was able to remove substantially all of the food impaction, or approximately two-thirds cup of semi-digested food material, and clear the esophagus, thereby also gaining complete visualization of the suspected esophageal stricture. Dilation was prescribed and administered to expand the esophagus in the affected area, and the patient experienced immediate and sustained relief with relatively minimal post-procedure discomfort.

### Example 3

### Foreign Object

This example demonstrates the use of an endoscope device according to aspects of the present invention in the non-surgical removal of a foreign object from the esophagus.

A 44-year-old male prisoner was taken to a local emergency room with digestive complications and vomiting, including blood in the regurgitation, and localized pain in the throat. An x-ray or radiographic evaluation was conducted, revealing what appeared to be a razor blade lodged in the patient's esophagus below the upper esophageal sphincter, apparently swallowed by the prisoner in an effort to conceal it or perhaps with the intent of being removed from the prison for medical intervention. The doctor on call sedated the patient and prepped for endoscopic removal of the razor blade.

Accordingly, a standard adult endoscope was selected along with a retriever attachment having a proximal end configured for frictional engagement on the distal end of the endoscope and a large distal lumen having a nominal inside diameter of 40 mm. The clinician selected the large retriever both because the device would not have to be intubated very far based on the location of the razor blade and because the intention was to have sufficient volume within which to pull the razor blade and shield it from the surrounding anatomy prior to any attempt to extubate. Based on the type and location of the foreign object the clinician did not want to attempt retrieval using known techniques involving graspers or forceps or a magnet for fear of esophageal perforation or trauma to the upper esophageal sphincter, further concluding that even the use of a hood would be inadequate in this case. With the patient and endoscope prepped, the clinician inserted the scope until the retriever was positioned within the upper esophageal sphincter just above the razor blade, the device conveniently holding the upper esophageal sphincter open and providing a wide work area for the clinician to manipulate a grasper tool through one of the channels of the scope, grasp the razor blade and slowly move it way from the wall of the esophagus and then draw it substantially completely within the distally-opening lumen of the retriever. With the razor blade secured in that position, the scope and device were withdrawn and the razor blade safely removed without any further damage to the patient's upper G.I. anatomy.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular apparatus, methodology, configuration, size, shape, material of construction, protocol, etc., described herein, but may include any such technology now known or later developed without departing from the spirit and scope of the specification. As such, various modifications or changes to or alternative configurations of the disclosed subject matter can be made in accordance with the teachings herein without departing from the spirit and scope of the present specification. Lastly, the terminology used herein is for the purpose of describing particular embodiments only, and is not intended to limit the scope of the present invention. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about”. As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An attachment for an endoscope having a suction tube extending to a distal end of the endoscope, the attachment comprising:
an endoscope receiving lumen having a proximal opening sized to receive in attachment the distal end of the endoscope; and
a blockage receiving lumen in communication with the endoscope receiving lumen and in fluid communication with the suction tube, the blockage receiving lumen having a distal opening opposing the proximal opening, the distal opening larger than a tube diameter of the suction tube and sized to receive at least a portion of an esophageal blockage drawn therethrough by suction generated through the suction tube, with at least a part of the portion of the esophageal blockage being stored within the blockage receiving lumen.

2. The attachment of claim 1 further comprising a body having an outer surface, the proximal opening located at a proximal end of the body and the distal opening located at a distal opening of the body.

3. The attachment of claim 2 wherein the outer surface tapers toward the distal opening.

4. The attachment of claim 2 wherein a wall is defined between the blockage receiving lumen and the outer surface, a wall thickness varying.

5. The attachment of claim 1 wherein at least one of the endoscope receiving lumen and the blockage receiving lumen are cylindrical.

6. The attachment of claim 1 wherein an endoscope stop is positioned within the endoscope receiving lumen to limit the distal travel of the distal end of the endoscope.

7. The attachment of claim 1 wherein a support shoulder is formed within one of the endoscope receiving lumen and the blockage receiving lumen, the support shoulder supporting a filter member that provides filtering between the distal opening to the proximal opening.

8. The attachment of claim 7 wherein the filter member bulges convexly toward the distal opening.

9. The attachment of claim 1 wherein a rim defines the distal opening, an annular projection extends inwardly from the rim.

10. The attachment of claim 9 wherein the annular projection rakes back toward the proximal opening to form a resilient funnel into the blockage receiving lumen.

11. The attachment of claim 1 wherein a wall is defined between the blockage receiving lumen and an outer surface, a tension element wraps at least partially about the blockage receiving lumen to draw the wall inwardly upon tensioning of the tensioning element.

12. The attachment of claim 11 wherein the tensioning element is a filament and is stitched about the wall or threaded through a casing that surrounds the blockage receiving lumen.

13. The attachment of claim 1 wherein the endoscope receiving lumen is substantially aligned with the blockage receiving lumen.

14. The attachment of claim 1 wherein a wall with a near portion and a far portion defines the blockage receiving lumen, the endoscope receiving lumen being smaller than and positioned within the blockage receiving lumen and abutting the near portion of the wall to define a proximal space between the endoscope receiving lumen and the far portion of the wall, a distal space being defined between the proximal space and the distal opening, the blockage receiving lumen comprising the proximal space and the distal space.

15. The attachment of claim 14 wherein a proximal channel opening opens to the proximal space, a suction channel fluidly communicates between the proximal channel opening and a distal channel opening.

16. An endoscopic instrument comprising:
an endoscope having a suction tube extending to a distal end of the endoscope; and
an endoscope device connected to the distal end of the endoscope lumen and in fluid communication with the suction tube, the endoscope device having a body with a blockage receiving lumen formed thereinto, the blockage receiving lumen having an opening larger than a tube diameter of the suction tube and sized to receive at least a portion of an esophageal blockage drawn therethrough by suction generated through the suction tube, with at least a part of the portion of the esophageal blockage being stored within the blockage receiving lumen.

17. The endoscopic instrument of claim 16 wherein the endoscope device is detachable from the distal end of the endoscope.

18. The endoscopic instrument of claim 16 wherein a wall with a near portion and a far portion defines the blockage receiving lumen, the distal end of the endoscope being positioned within the blockage receiving lumen and abutting the near portion of the wall to define a proximal space between the endoscope receiving lumen and the far portion of the wall.

19. The attachment of claim 18 wherein a proximal channel opening opens to the proximal space, a suction channel fluidly communicates between the proximal channel opening and a distal channel opening.

20. An attachment for an endoscope having a suction tube extending to a distal end of the endoscope, the attachment comprising:
a body having a proximal end and a far end;
an endoscope receiving means for receiving a the distal end of the endoscope at the proximal end of the body; and
a blockage receiving means for receiving at least a portion of an esophageal blockage drawn therethrough by suction generated through the suction tube, the blockage receiving means having a distal opening larger than a tube diameter of the suction tube.
